SEQUENCE LISTING (3 sequences)

SEQ ID NO: 1 - protein, 354 aa, Homo sapiens
MSLRGSLSRLLQTRVHSILKKSVHSVAVIGAPFSQGQKRKGVEHGPAAIREAGLMKRLSSLGCHLKDFGDLSFTPVPKDDLYNNLIVNPRSVGLANQELAEVVSRAVSDGYSCVTLGGDHSLAIGTISGHARHCPDLCVVWVDAHADINTPLTTSSGNLHGQPVSFLLRELQDKVPQLPGFSWIKPCISSASIVYIGLRDVDPPEHFILKNYDIQYFSMRDIDRLGIQKVMERTFDLLIGKRQRPIHLSFDIDAFDPTLAPATGTPVVGGLTYREGMYIAEEIHNTGLLSALDLVEVNPQLATSEEEAKTTANLAVDVIASSFGQTREGGHIVYDQLPTPSSPDESENQARVRI

SEQ ID NO: 2 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 3 - protein, 321 aa, Homo sapiens
SAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

(19) United States
(12) Patent Application Publication
(10) Pub. No.: US 2025/0228921 A1
(43) Pub. Date: Jul. 17, 2025

(54) TREATMENT OF ARGINASE 1 DEFICIENCY

(71) Applicant: Immedica Pharma AB, Stockholm (SE)

(72) Inventor: Anthony G. Quinn, Austin, TX (US)

(21) Appl. No.: 18/854,456

(22) PCT Filed: Apr. 11, 2023
(86) PCT No.: PCT/US2023/065630

§ 371 Date: Oct. 4, 2024

Related U.S. Application Data
(60) Provisional application No. 63/329,867, filed on Apr. 11, 2022.

(51) Int. Cl. A61K 38/50; A61K 9/00; A61K 31/047; A61K 31/192; A61K 31/225; A61K 33/14; A61K 33/42; A61P 3/00

(52) U.S. Cl. CPC A61K 38/50; A61K 9/0019; A61K 31/047; A61K 31/192; A61K 31/225; A61K 33/14; A61K 33/42; A61P 3/00; C12Y 305/03001

(57) ABSTRACT

A method of treating Arginase 1 (ARG1) deficiency (ARG1-D) in a subject, comprising administering a pegzilarginase to the subject, wherein the pegzilarginase is a pegylated human arginase 1 comprising a cobalt metal cofactor, and wherein the pegzilarginase is administered weekly at a dose of from about 0.05 mg/kg to about 0.2 mg/kg.

### CROSS-REFERENCE TO RELATED APPLICATIONS

This application claims priority to PCT Application No. PCT/US2023/065630, filed Apr. 11, 2023, and to U.S. Provisional No. 63/329,867, filed Apr. 11, 2022, each of which is incorporated herein by reference in its entirety.

### TECHNICAL FIELD

The present disclosure generally relates to the field of compositions comprising human arginase 1. Methods of using the compositions are also provided herein.

### SEQUENCE LISTING

The contents of the electronic sequence listing (ZACC285001APC.xml; Size: 4,658 bytes; and Date of Creation: Oct. 4, 2024) are herein incorporated by reference in its entirety.

### BACKGROUND

ARG1-D is a rare, progressive, multisystem, autosomal recessive disease (Summar 2013, Diez-Fernandez 2018, Schlune 2015, Waisbren 2018). This disease typically presents in early childhood and is caused by deficiency in the enzyme arginase 1 (ARG1 [EC 3.5.3.1]), which leads to two important harmful metabolic effects: accumulation of high levels of arginine and arginine-derived metabolites, and impairment of the urea cycle, which leads to episodic elevation of ammonia levels.

The high plasma arginine level is believed to be the key driver of spasticity, developmental delays, and seizures, which develop in early childhood and progress over a lifetime (De Deyn 1997, Waisbren 2018). The lower-limb spasticity in early childhood impairs mobility and balance, leading to difficulties in walking and climbing stairs and lack of independence. School performance and educational achievement are markedly impacted by developmental delays and cognitive decline. The neuromotor and neurocognitive effects result from persistently elevated arginine levels and profoundly impact daily functioning and quality of life at an early age, progressively worsening over time, leading to severe disabilities and early death (De Deyn 1997, Oeffinger 2008, Prasad 1997, Carvalho 2012).

In addition to the severe neuromotor and neurocognitive manifestations, which dominate the clinical picture, subjects with ARG1-D manifest other medically important disease-related abnormalities, including complications due to hyperammonemia, hepatocellular injury, inadequate nutrition, and growth impairment. These abnormalities are a result of or are associated with the complications of the disease or the required treatment with severe protein restriction.

The goal for long-term management of ARG1-D is to reduce plasma arginine levels without adversely impacting growth and development. Current disease management includes severe protein restriction and EAA supplementation to lower arginine, with or without the use of ammonia scavengers to prevent or treat hyperammonemia. Severe dietary protein restriction can modestly lower plasma arginine levels with a degree of improvement in disease-related abnormalities, thus supporting the value of arginine reduction.

However, this approach is inadequate as most patients continue to have marked hyperargininemia. Moreover, the diet is challenging, particularly in children, and requires supplementation with unpalatable EAA formulas to maintain adequate essential amino acid intake (Haberle 2012, Huemer 2016, Lambert 1991, Burrage 2015). The progressive nature of this disease, despite current approaches, highlights the important unmet need for a therapy that will lower arginine levels beyond those achievable with current standard disease management to current guidelines or even to normal levels and potentially slow or halt the progression of neuromotor and/or neurocognitive deterioration in patients with ARG1-D.

### SUMMARY

Delivery of Arginase 1 Deficiency treatment using cobalt substituted Arginase 1 alone or in combination with one more other therapies is described. The other therapy delivered in combination with Arginase 1 therapy may be, for example, diet restriction and/or ammonia scavengers. A combination therapy that includes Arginase 1 may be more able to completely address complex or multifactorial disease.

In one aspect, provided herein are methods of treating Arginase 1 (ARG1) deficiency (ARG1-D) in a subject, comprising administering a pegzilarginase to the subject, wherein the pegzilarginase is a pegylated human arginase 1 comprising a cobalt metal cofactor, and wherein the pegzilarginase is administered weekly at a dose of from about 0.05 mg/kg to about 0.2 mg/kg.

In one aspect, provided herein are methods of treating Arginase 1 (ARG1) deficiency (ARG1-D) in a subject, comprising administering a human arginase 1 to the subject, wherein the human arginase 1 is a pegylated human arginase 1 comprising a cobalt metal cofactor, and wherein the human arginase 1 is administered weekly at a dose of from about 0.05 mg/kg to about 0.2 mg/kg.

In one aspect, provided herein is a combination therapy for the treatment of Arginase 1 Deficiency, particularly to a combination of (i) a cobalt substituted arginase or fragment thereof capable of reducing arginine in blood to within a normal range and any one or more of (ii) diet restriction and (iii) ammonia scavengers.

In one aspect, provided herein is treatment of Arginase 1 Deficiency using cobalt substituted Arginase 1, with or without another agent in combination.

The present disclosure also relates to subsets of Arginase 1 Deficiency patients that display particular manifestations of disease or particular symptoms.

The present disclosure also relates to preventing arginine metabolite accumulation.

The present disclosure also relates to diet modification (including, but not limited to, eliminating or reducing dietary restrictions) in patients having Arginase 1 Deficiency that are treated with cobalt substituted Arginase 1.

### BRIEF DESCRIPTION OF FIGURES

FIG. 1A shows the clinical study design schema. FIG. 1B shows the key demographics and baseline clinical characteristics.

FIG. 2 shows the waterfall plot of arginine (μM) change from baseline at week 24 during the double-blind period.

FIG. 3A shows the box plot of arginine levels (μM) over time during the double-blind period. FIG. 3B shows the percentage of patients achieving normalization of plasma arginine at different time points.

FIG. 4A shows the 2-minute walk test (meters) change from baseline at week 24 during the double-blind period for each group. FIG. 4B shows the waterfall plot of 2-minute walk test (meters) change from baseline at week 24 during the double-blind period for each individual.

FIG. 5A shows the GMFM-E change from baseline at week 24 during the double-blind period for each group. FIG. 5B shows the waterfall plot of GMFM-E change from baseline at week 24 during the double-blind period for each individual.

FIG. 6 shows the box plot of ornithine (μM) over time during the double-blind period.

FIG. 7 shows the box plot of Alpha-N-Acetylarginine (μM) over time during the double-blind period.

FIG. 8 shows the box plot of Alpha-keto-d-Guanidinovaleric Acid (μM) over time during the double-blind period.

FIG. 9 shows the box plot of Argininic Acid (μM) over time during the double-blind period.

FIG. 10 shows the box plot of Guanidinoacetic Acid (μM) over time during the double-blind period.

FIG. 11 shows the waterfall plot of GMFM-D change from baseline at week 24 during the double-blind period.

FIG. 12 shows the responder heat map for evaluable subjects.

FIG. 13 shows the selected values from FAO/WHO/UNU safe levels of protein intake and energy requirements of children and adults, as well as during pregnancy and lactation, for the healthy population.

FIG. 14 shows the summary of changes from baseline at week 24 in other secondary efficacy endpoints.

FIG. 15 shows the dosing strategies tested for IV and SC administration during the adaptive dosing simulations.

FIG. 16 shows the distribution of final doses via intravenous route.

FIG. 17 shows the distribution of final doses via subcutaneous route.

FIG. 18 shows the relationship between baseline L-arginine and final dose.

FIG. 19 shows the number of dose titrations for intravenous route.

FIG. 20 shows the differences between IV and SC dosing.

FIG. 21 shows the L-arginine time in recommended range.

### OTHER MATERIALS

The following patents and applications are incorporated by reference herein in their entireties: U.S. Pat. No. 8,440,184; US Reissue Pat. No. RE46,423; US Pat. App. Pub. No. 2019/0167770; and US Pat. App. Pub. No. 2021/0189371.

### DETAILED DESCRIPTION

Disclosed herein are methods of treating Arginase 1 (ARG1) deficiency (ARG1-D) in a subject, comprising administering a pegzilarginase or a human arginase 1 to the subject, wherein the pegzilarginase or the human arginase 1 is a pegylated human arginase 1 comprising a cobalt metal cofactor, and wherein the pegzilarginase or the human arginase 1 is administered weekly at a dose of from about 0.05 mg/kg to about 0.2 mg/kg. In embodiments, the subject's plasma arginine level is between about 40 μM and about 115 μM after the treatment. In embodiments, patients with GMFCS classifications of ≥II demonstrate greater improvements in mobility after the treatment. In embodiments, the initial dose of the pegzilarginase or the human arginase 1 is about 0.1 mg/kg. In embodiments, the subsequent doses of the pegzilarginase or the human arginase 1 are adjusted based on the subject's plasma arginine level prior to the administration of the subsequent doses.

### Definitions

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa unless the content clearly dictates otherwise. In the event that any description of a term set forth conflicts with any document incorporated herein by reference, the description of the term set forth below shall control.

As used herein, the singular forms “a”, “an” and “the” include plural references unless the context clearly dictates otherwise.

As used herein, the term “and/or” is used in this disclosure to either “and” or “or” unless indicated otherwise.

As used herein, “about”, “approximately”, “substantially”, and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10%, 5%, or 1% of the particular term and “substantially” and “significantly” will mean more than plus or minus 10% of the particular term.

The term “administering” refers the injection of a therapeutically effective amount of the compound and compositions containing said compound disclosed. For example, without limitation, administration can be intravascular (i.v.) or subcutaneous (s.c.). The compositions of the invention can also be administered intramuscularly (i.m.). The compound can be the pegzilarginase or the human arginase 1 disclosed herein. The composition can be the composition comprising the pegzilarginase or the human arginase 1 disclosed herein.

The term “diet liberalization” refers to a change in dietary intake for an Arg-1 D patient that is on any type of diet restriction as part of disease management, including addition of any food item that is not an arginine-free (or low arginine) essential amino acid mixture.

The term “effective amount” refers to that amount of a wild-type arginase, such as human arginase 1, or a pegylated arginase, such as pegzilarginase, being administered that will have the desired effect, such reducing a plasma level of one or more of the following: arginine, argininic acid (ArgA), GVA, N-a-acetylarginine (NAArg), GAA, and homoarginine (HArg). One example would be to administer a compound to achieve a range arginine, ArgA, GVA, GAA, and/or NAArg that is equivalent to a normal level of each compound in a normal human who does not suffer from ARG1 deficiency. Effective amounts can also improve muscle strength, ambulatory ability of a patient (i.e., ability to run, walk, ride a bike, climb stairs without support), and improve cognitive ability (for example Wechsler Intelligence Scale for Children (WISC) testing improvement) and/or adaptive behavior (for example Adaptive Behavior Assessment Scale (ABAS) or Vineland Adaptive Behavior Scale (VABS) testing improvement) (Lopata et al., “Comparison of Adaptive Behavior Measures for Children with HFASDs”, Autism Researc and Treatment, Vol. 2013, pp. 1-10, (2013)). A normal level of arginine is described by Luneburg, N. et al., (2011). The effective amount may vary with factors such as the weight of the patient.

The term “Gross Motor Function Classification System” or “GMFCS” scale refers to the criteria for GMFCS Levels I-V are provided below.

### GMFCS Level I

Can walk indoors and outdoors and climb stairs without using hands for support
Can perform usual activities such as running and jumping
Has decreased speed, balance and coordination.

### GMFCS Level II

Has the ability to walk indoors and outdoors and climb stairs with a railing
Has difficulty with uneven surfaces, inclines or in crowds
Has only minimal ability to run or jump.

### GMFCS Level III

Walks with assistive mobility devices indoors and outdoors on level surfaces
May be able to climb stairs using a railing
May propel a manual wheelchair (may require assistance for long distances or uneven surfaces).

### GMFCS Level IV

Walking ability severely limited even with assistive devices
Uses wheelchairs most of the time and may propel their own power wheelchair
May participate in standing transfers.

### GMFCS Level V

Has physical impairments that restrict voluntary control of movement and the ability to maintain head and neck position against gravity
Is impaired in all areas of motor function
Cannot sit or stand independently, even with adaptive equipment
Cannot independently walk, though may be able to use powered mobility.

The term “Gross Motor Function Measure” or “GMFM” scale refer to clinical measure designed to evaluate gross motor function by observing the subject's ability to initiate and complete certain movements. There are two versions of the GMFM. The GMFM-88 is the original 88-item measure. Items span the spectrum of gross motor activities in five dimensions: A: Lying and Rolling, B: Sitting, C: Crawling and Kneeling, D: Standing, and E: Walking, Running and Jumping.

The GMFM-66 is a 66 item subset of the original 88 items identified through Rasch analysis to best describe the gross motor function of children with cerebral palsy of varying abilities. It has a unidimensional scale providing interval scaling rather than the ordinal scaling of the GMFM-88. Items are ordered in terms of difficulty and a unit of change has the same meaning throughout the scale ranging from 0 to 100. The GMFM-66 provides information on the level of difficulty of each item thereby providing information to assist with realistic goal setting.

The term “Individualized Disease Management” refers to the treatment given to a particular ARG1-D patient based upon the treating physician's assessment, which typically included a prescribed diet with severe protein restriction and essential amino acid (EAA) supplementation and/or the use of ammonia scavengers.

The term “natural protein” refers to any protein that is not a mixture of only essential amino acids, including beans, legumes, lentils, grains, bread, vegetables, fish, meat, eggs, milk, whey protein, yogurt, cheese, nuts and other food consumed as a nutritional diet.

The term “pegylated” refers to conjugation with polyethylene glycol (PEG), which has been widely used as a drug carrier, given its high degree of biocompatibility and ease of modification (see e.g., Harris et al, Clin. Pharmacokinet. 40 (7): 539-51, 2001). PEG can be coupled (e.g., covalently linked) to active agents through the hydroxyl groups at the ends of the chain and via other chemical methods; however, PEG itself is limited to at most two active agents per molecule. In a different approach, copolymers of PEG and amino acids have been explored as novel biomaterials which would retain the biocompatibility properties of PEG, but which would have the added advantage of numerous attachment points per molecule (providing greater drug loading). The PEGylated arginase variants can be formulated according to known methods to prepare pharmaceutically useful compositions. An ARG1-D patient can be administered a wild-type arginase protein (either arginase I or arginase II) that naturally contains manganese metal cofactor, or a wild-type arginase protein that has been pegylated and contains manganese metal cofactor. In another example, the ARG1-D patient can be administered an arginase that has cobalt metal cofactor in lieu of the native manganese metal cofactor. The cobalt metal cofactor containing arginase can further be pegylated, with an exemplary form being Co-Arg1-PEG (also referred to herein as AEB1 102, pegzilarginase, or Co-hArgI) such as that described in U.S. Pat. No. 8,440,184, incorporated herein by reference in its entirety. An exemplary form, such as pegzilarginase has approximately twelve 5 K (5000 Dalton) PEG units per monomer attached to one or more lysines present in the protein sequence of pegzilarginase.

The term “pharmaceutical or pharmacologically acceptable” refers to molecular entities and compositions that do not produce an adverse, allergic or other untoward reaction when administered to a subject, such as a human, as appropriate. The preparation of a pharmaceutical composition that contains at least one arginase variant, such as a stabilized multimeric arginase or a pegylated arginase isolated by the method disclosed herein, or additional active ingredient will be known to those of skill in the art in light of the present disclosure, as exemplified by REMINGTON'S PHARMACEUTICAL SCIENCES, 18th Ed., 1990. Moreover, for animal (e.g., human) administration, it will be understood that preparations should meet sterility, pyrogenicity, general safety and purity standards as required by FDA Office of Biological Standards.

The term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, dyes, such like materials and combinations thereof, as would be known to one of ordinary skill in the art (see e.g., REMINGTON'S PHARMACEUTICAL SCIENCES, 18th Ed., 1990). Except insofar as any conventional carrier is incompatible with the active ingredient, its use in the pharmaceutical compositions is contemplated.

The term “primary source” refers to a substance that constitutes at least 50% of the total source. For instance, the primary source of a subject's protein intake is natural protein means that natural protein constitutes at least 50% of the source of the subject's protein intake.

The terms “protein” and “polypeptide” refer to compounds comprising amino acids joined via peptide bonds and are used interchangeably.

The term “subject” refers to animals such as mammals, including humans.

The terms “treating”, “to treat”, or “treatment”, include restraining, slowing, stopping, reducing, ameliorating, or reversing the progression or severity of an existing symptom, disorder, condition, or disease relating to ARG1-D. A treatment may be applied prophylactically or therapeutically.

### Abbreviations

| Abbreviation | Definition |
|---|---|
| 2MWT | 2-Minute Walk Test |
| ADA | anti-drug antibody |
| AE | adverse event |
| AESI | adverse event of special interest |
| ALP | alkaline phosphatase |
| ALT | alanine aminotransferase |
| ANCOVA | analysis of covariance |
| aPTT | activated partial thromboplastin time |
| ARG1-D | arginase 1 deficiency |
| AST | aspartate aminotransferase |
| BSID-III | Bayley Scales of Infant and Toddler Development, |
| | Third Edition |
| BUN | blood urea nitrogen |
| CDC | Centers for Disease Control and Prevention |
| COVID-19 | coronavirus disease of 2019 |
| CRA | clinical research associate |
| CS | clinically significant |
| CSR | clinical study report |
| DB | double-blind |
| DNA | deoxyribonucleic acid |
| EAA | essential amino acid |
| ECG | electrocardiogram |
| eCRF | electronic case report form |
| EDC | electronic data capture |
| EEG | electroencephalogram |
| EU | European Union |
| FMA | Functional Mobility Assessment |
| FMS | Functional Mobility Scale |
| GCP | Good Clinical Practice |
| GFAQ | Gillette Functional Assessment Questionnaire |
| GMFCS | gross motor function classification system |
| GMFM-88 | Gross Motor Function Measure-88 |
| GMFM-D | Gross Motor Function Measure-88 Part D |
| GMFM-E | Gross Motor Function Measure-88 Part E |
| HLT | High Level Term |
| ICF | informed consent form |
| ICH | International Council for Harmonisation of Technical |
| | Requirements for Pharmaceuticals for Human Use |
| IDM | individualized disease management |
| IEC | Independent Ethics Committee |
| IgE | immunoglobulin E |
| IGF-1 | insulin-like growth factor 1 |
| INR | international normalized ratio |
| IP | investigational product |
| IRB | Institutional Review Board |
| IV | intravenous(ly) |
| IQ | intelligence quotient |
| IXRS | interactive web/voice response system |
| LS | least squares |
| LTE | long-term extension |
| MAR | missing-at-random |
| MAS | Modified Ashworth Scale |
| MedDRA | Medical Dictionary for Regulatory Activities |
| MMRM | mixed effect model with repeated measures |
| NCS | not clinically significant |
| NIH | National Institutes of Health |
| NOS | not otherwise specified |
| PD | pharmacodynamic(s) |
| PedsQL | Pediatric Quality of Life Inventory |
| PEG | polyethylene glycol |
| PK | pharmacokinetic(s) |
| QW | weekly |
| RBC | red blood cell |
| SAE | serious adverse event |
| SAP | Statistical Analysis Plan |
| SC | subcutaneous(ly) |
| SF-36 | 36-Item Short Form Health Survey |
| SOC | system organ class |
| sRBP | serum retinol binding protein |
| SRC | Safety Review Committee |
| TEAE | treatment-emergent adverse event |
| TIBC | total iron-binding capacity |
| UK | United Kingdom |
| ULN | upper limit of normal |
| US | United States |
| VABS-II | Vineland Adaptive Behavior Scale, Second Edition |
| WAIS-IV | Wechsler Adult Intelligence Scale, Fourth Edition |
| WISC-V | Wechsler Intelligence Scale for Children, Fifth Edition |
| WPPSI-IV | Wechsler Preschool and Primary Scale of Intelligence, |
| | Fourth Edition |
| ZBI-12 | 12-Item Short Form Zarit Burden Interview |

### Arginases

In one aspect, disclosed herein are arginases. Wild-type arginase is a manganese-containing enzyme. It is the final enzyme of the urea cycle. Arginase is the fifth and final step in the urea cycle, a series of biophysical reactions in mammals during which the body disposes of harmful ammonia. Specifically, arginase converts L-arginine into L-omithine and urea.

L-Arginine is the nitrogen donating substrate for nitric oxide synthase (NOS), producing L-citrulline and nitric oxide (NO). Although the KM of arginase (2-5 mM) has been reported to be much higher than that of NOS for L-Arginine (2-20 mM), arginase may also play a role in regulating NOS activity. Under certain conditions Arginase I is Cys-S-nitrosylated, resulting in higher affinity for L-Arginine and reduced availability of substrate for NOS.

Arginase is a homo-trimeric enzyme with an a/b-fold of a parallel eight-stranded b-sheet surrounded by several helices. The enzyme contains a di-nuclear metal cluster that is integral to generating a hydroxide for nucleophilic attack on the guanidinium carbon of L-Arginine. The native metal cofactor for Arginase is Mn²⁺. These Mn²⁺ ions coordinate water, orientating and stabilizing the molecule and allowing water to act as a nucleophile and attack L-arginine, hydrolyzing it into ornithine and urea.

Mammals have two Arginase isozymes (EC 3.5.3.1) that catalyze the hydrolysis of L-Arginine to urea and L-ornithine. The Arginase I gene is located on chromosome 6 (6923), is highly expressed in the cytosol of hepatocytes, and functions in nitrogen removal as the final step of the urea cycle. The Arginase II gene is found on chromosome 14 (14q24.1). Arginase II is mitochondrially located in tissues such as kidney, brain, and skeletal muscle where it is thought to provide a supply of L-Ornithine for proline and polyamine biosynthesis (Lopez et al, FEBSJ. 272:4540-48, 2005).

Wild type human Arginase II has the following sequence (Uniprot P78540):

(SEQ ID NO: 1)
MSLRGSLSRLLQTRVHSILKKSVHSVAVIGAPFSQGQKRKGVEHGPAAIR
EAGLMKRLSSLGCHLKDFGDLSFTPVPKDDLYNNLIVNPRSVGLANQELA
EVVSRAVSDGYSCVTLGGDHSLAIGTISGHARHCPDLCVVVDAHADINTP
LTTSSGNLHGQPVSFLLRELQDKVPQLPGFSIKPCISSASIVYIGLRDVD
PPEHFILKNYDIQYFSMRDIDRLGIQKVMERTFDLLIGKRQRPIHLSFDI
DAFDPTLAPATGTPVVGGLTYREGMYIAEEIHNTGLLSALDLVEVNPQLA
TSEEEAKTTANLAVDVIASSFGQTREGGHIVYDQLPTPSSPDESENQARV
RI

Wild type human Arginase I has the following sequence (Uniprot/P05089):

(SEQ ID NO: 2)
MSAKSRTIGIIGAPFSKGQPRGGVEEGPTVLRKAGLLEKLKEQECDVKDY
GDLPFADIPNDSPFQIVKNPRSVGKASEQLAGKVAEVKKNGRISLVLGGD
HSLAIGSISGHARVHPDLGVIWVDAHTDINTPLTTTSGNLHGQPVSFLLK
ELKGKIPDVPGFSWVTPCISAKDIVYIGLRDVDPGEHYILKTLGIKYFSM
TEVDRLGIGKVMEETLSYLLGRKKRPIHLSFDVDGLDPSFTPATGTPVVG
GLTYREGLYITEEIYKTGLLSGLDIMEVNPSLGKTPEEVTRTVNTAVAIT
LACFGLAREGNHKPIDYLNPPK

Wild type human Arginase I without the N-terminal methionine has the following sequence:

(SEQ ID NO: 3)
SAKSRTIGIIGAPFSKGQPRGGVEEGPTVLRKAGLLEKLKEQECDVKDYG
DLPFADIPNDSPFQIVKNPRSVGKASEQLAGKVAEVKKNGRISLVLGGDH
SLAIGSISGHARVHPDLGVIWVDAHTDINTPLTTTSGNLHGQPVSFLLKE
LKGKIPDVPGFSWVTPCISAKDIVYIGLRDVDPGEHYILKTLGIKYFSMT
EVDRLGIGKVMEETLSYLLGRKKRPIHLSFDVDGLDPSFTPATGTPVVGG
LTYREGLYITEEIYKTGLLSGLDIMEVNPSLGKTPEEVTRTVNTAVAITL
ACFGLAREGNHKPIDYLNPPK

Arginases have been investigated for nearly 50 years as a method for degrading extracellular L-Arginine (Dillon et al, “Biochemical characterization of the arginine degrading enzymes arginase and arginine deiminase and their effect on nitric oxide production”, Med. Sci. Monit., 8 (7): BR248-253 (2002)). While native arginase is cleared from circulation within minutes (Savoca et al., Cancer Biochem. Biophys. 7:261-268, 1984), a single injection of PEG-Arginase MW 5,000 in rats was sufficient to achieve near complete arginine depletion for about 3 days (Cheng et al., Cancer Res. 67:309-17, 2007).

A bacterial arginine hydrolyzing enzyme, ADI, which displays good kinetics and stability, has been tested in vitro. Unfortunately, ADI is a bacterial enzyme and therefore it induces strong immune responses and adverse effects in most patients and is not suitable for long-term administration in ARG1-D patients who would require regular administration.

For clinical use in patients with ARG1-D, it is essential that an arginase is engineered to allow it to persist for long times (e.g., days) in circulation. In the absence of any modification, human arginase has a half-life of only a few minutes in circulation primarily because its size is not sufficiently large to avoid filtration though the kidneys. Unmodified human arginase is very susceptible to deactivation in serum, and it is degraded with a half-life of only four hours. In embodiments, the human arginase is human arginase 1. In embodiments, the human arginase 1 is pegylated. In embodiments, the human arginase 1 has a cobalt metal cofactor. In embodiments, the human arginase 1 is pegylated and has a cobalt metal cofactor. In embodiments, the human arginase 1 comprises the amino acid sequence of SEQ ID NO: 2. In embodiments, the human arginase 1 comprises the amino acid sequence of SEQ ID NO: 3. In embodiments, the human arginase 1 comprises at least one amino acid substitution at a metal binding site and/or is truncated by one or more amino acids in reference to SEQ ID NO: 2 or 3. In embodiments, the human arginase 1 is pegylated, has a cobalt metal cofactor, and comprises the amino acid sequence of SEQ ID NO: 2. In embodiments, the human arginase 1 is pegylated, has a cobalt metal cofactor, and comprises the amino acid sequence of SEQ ID NO: 3. In embodiments, the human arginase 1 is pegylated, has a cobalt metal cofactor, and comprises at least one amino acid substitution at a metal binding site and/or is truncated by one or more amino acids in reference to SEQ ID NO: 2 or 3.

In embodiments, a pegzilarginase disclosed herein has the sequence of Arginase I, SEQ ID NO: 2, and has a cobalt metal cofactor in lieu of a manganese metal cofactor. In embodiments, a pegzilarginase disclosed herein has the sequence of Arginase I, SEQ ID NO: 3, and has a cobalt metal cofactor in lieu of a manganese metal cofactor. In embodiments, the pegzilarginase is pegylated as described in U.S. Pat. No. 8,440,184 and manufactured as described in US Pat. App. Pub. No. 2021/0189371.

In aspects of the disclosure, methods and compositions related to pegylated arginase are disclosed. Specifically, PEGylation of arginase at an engineered Cysteine residue (e.g., substituting the third residue of the N-terminal) may be used to produce a homogenous pegylated arginase composition. Methods for isolation of pegylated arginase based on temporary disruption of polymerization are also disclosed.

PEGylation is the process of covalent attachment of PEG polymer chains to another molecule, normally a drug or therapeutic protein. “PEGylation” can be achieved by incubation of a reactive derivative of PEG with the target macromolecule. An increase of the hydrodynamic size (size in solution) of the drug or therapeutic protein prolongs its circulatory time by reducing renal clearance. PEGylation can also provide water solubility to hydrophobic drugs and proteins. The human arginase 1, including pegzilarginase, disclosed herein can be pegylated as described in U.S. Pat. No. 8,440,184.

The first step in PEGylation can be the suitable functionalization of the PEG polymer at one or both terminal domains of the protein or internally to amino acids, such as lysines. PEGs that are activated at each terminus with the same reactive moiety are known as “homobifunctional”, whereas if the functional groups present are different, then the PEG derivative is referred as “heterobifunctional” or “heterofunctional”. The chemically active or activated derivatives of the PEG polymer are prepared to attach the PEG to the desired molecule.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid can also be used to attach the PEG to the polypeptide.

The techniques used to form PEG derivatives include reacting the PEG polymer with a group that is reactive with hydroxyl groups, typically anhydrides, acid chlorides, chloroformates, and carbonates. PEGylation chemistry can also use functional groups such as aldehyde, esters, amides etc. made available for conjugation. Heterobifunctional PEGs are very useful in linking two entities, where a hydrophilic, flexible and biocompatible spacer is needed. Preferred end groups for heterobifunctional PEGs are maleimide, vinyl sulfones, pyridyl disulfide, amine, carboxylic acids, and H-hydroxysuccinimide (NHS) esters.

The most common modification agents, or linkers, are based on methoxy polyethylene glycol (mPEG) molecules. Their activity depends on adding a protein-modifying group to the alcohol end. Polyethylene glycol (PEG diol) can be used as the precursor molecule; the diol is subsequently modified at both ends in order to make a hetero- or homo-dimeric PEG-linked molecule.

Proteins are generally PEGylated at nucleophilic sites such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The amide formed with the maleimides can be somewhat unstable under alkaline conditions so there may be some limitation to formulation options with this linker. The amide linkage formed with iodo-PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo-PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. These amine-specific agents generally react under mild conditions and are very specific for amino groups.

Due to the multiple lysine residues on most proteins, site-specific PEGylation can be a challenge. Fortunately, because these reagents react with unprotonated amino groups, it is possible to direct the PEGylation to lower-pK amino groups by performing the reaction at a lower pH. Generally, the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a pegylation procedure. Fortunately, there are usually no more than four or five key parameters. The “design of experiments” approach to optimization of pegylation conditions can be very useful. For thiol-specific pegylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the pegylation reaction. For example, if the pegylation agent is only 70 percent active, the amount of PEG used should ensure that only active PEG molecules are counted in the protein-to-PEG reaction stoichiometry.

### Pharmaceutical Compositions

In one aspect, disclosed herein are pharmaceutical compositions comprising an arginase. In embodiments, the arginase is a pegzilarginase, which is a pegylated human arginase 1 comprising a cobalt metal cofactor. In embodiments, the arginase is a human arginase 1. In embodiments, the human arginase 1 is pegylated and has a cobalt metal cofactor.

The arginases described herein and compositions comprising them can be administered systemically or locally. The arginases and compositions comprising them can be administered intravenously, intrathecally, subcutaneously, intramuscularly, intratumorally, and/or intraperitoneally or a combination thereof. The arginases described herein and compositions comprising them can be administered alone or in combination with ammonia scavengers, such as sodium phenylbutyrate, sodium benzoate, and glycerol phenylbutyrate.

Compositions containing an arginase or portion thereof can be provided in formulations together with physiologically tolerable liquid, gel or solid carriers, diluents, and excipients. Such compositions are typically prepared as liquid solutions or suspensions, as injectables. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH buffering agents. Where clinical applications are contemplated, it may be necessary to prepare pharmaceutical compositions-expression vectors, virus stocks, proteins, antibodies and drugs-in a form appropriate for the intended application. Generally, pharmaceutical compositions of the present invention comprise an effective amount of one or more arginase variants or additional agent dissolved or dispersed in a pharmaceutically acceptable carrier.

The pharmaceutical compositions containing arginase may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intramuscularly, subcutaneously, intratumorally, locally, injection, infusion, continuous infusion, via a catheter, in lipid compositions (e.g., liposomes), or by other method or any combination of the forgoing as would be known to one of ordinary skill in the art (see, e.g., REMINGTON'S PHARMACEUTICAL SCIENCES).

The arginase variants may be formulated into a composition in a free base, neutral or salt form. Pharmaceutically acceptable salts include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions can be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

The compositions for administration can be provided in a pharmaceutically acceptable carrier with or without an inert diluent. The carrier should be capable of assimilating and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate.

Examples of carriers or diluents include fats, oils, water, saline solutions, lipids, liposomes, resins, binders, fillers and the like, or combinations thereof. The composition may also comprise various antioxidants to retard oxidation of one or more component. Additionally, the use of preservatives such as various antibacterial and antifungal agents, including but not limited to parabens (e.g., methylparabens, propylparabens), chlorobutanol, phenol, sorbic acid, thimerosal or combinations thereof may improve longevity of the pharmaceutical composition.

The composition can be combined with the carrier in any convenient and practical manner, i.e., by solution, suspension, emulsification, admixture, encapsulation, absorption and the like. Such procedures are routine for those skilled in the art.

A pharmaceutical lipid vehicle can be used for the compositions that include arginase variants. The lipid vehicle compositions can comprise one or more lipids, and an aqueous solvent. As used herein, the term “lipid” refers to include any of a broad range of substances that is characteristically insoluble in water and extractable with an organic solvent. Examples include compounds, which contain long-chain aliphatic hydrocarbons and their derivatives. A lipid may be naturally occurring or synthetic (i.e., designed or produced by man). However, a lipid is usually a biological substance.

Biological lipids include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

The actual dosage amount of a composition comprising an arginase described herein to a patient can be determined by physical and physiological factors such as body weight, severity of condition, the type of disease being treated, previous or concurrent therapeutic interventions, idiopathy, of the patient and on the route of administration.

Depending upon the dosage and the route of administration, the number of administrations of a preferred dosage and/or an effective amount may vary by subject. The dosage will depend on the amount needed for the patient to achieve normal levels of at least one or more of the following: arginine, HArg, ArgA, GVA, GAA, and NAArg. ARG1-D Patient levels of one or more of these five (5) compounds are generally assessed until normal ranges are obtained in the plasma of the patient. Tissue levels of the five (5) compounds can also be assessed, but may not be necessary or may be performed less frequently than plasma level testing.

A pharmaceutical composition may comprise, for example, at least about 0.1% of an active compound. In embodiments, the active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, and other pharmacological considerations will be contemplated by one skilled in the art of preparing such pharmaceutical formulations, and as such, a variety of dosages and treatment regimens may be desirable.

In embodiments, the pegzilarginase is stored at the concentration of about 1 mg/mL to about 8 mg/mL. In embodiments, the pegzilarginase is stored at the concentration of at least about 1 mg/mL. In embodiments, the pegzilarginase is stored at the concentration of at most about 8 mg/mL. In embodiments, the pegzilarginase is stored at the concentration of about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 6 mg/mL, about 1 mg/mL to about 7 mg/mL, about 1 mg/mL to about 8 mg/mL, about 2 mg/mL to about 3 mg/mL, about 2 mg/mL to about 4 mg/mL, about 2 mg/mL to about 5 mg/mL, about 2 mg/mL to about 6 mg/mL, about 2 mg/mL to about 7 mg/mL, about 2 mg/mL to about 8 mg/mL, about 3 mg/mL to about 4 mg/mL, about 3 mg/mL to about 5 mg/mL, about 3 mg/mL to about 6 mg/mL, about 3 mg/mL to about 7 mg/mL, about 3 mg/mL to about 8 mg/mL, about 4 mg/mL to about 5 mg/mL, about 4 mg/mL to about 6 mg/mL, about 4 mg/mL to about 7 mg/mL, about 4 mg/mL to about 8 mg/mL, about 5 mg/mL to about 6 mg/mL, about 5 mg/mL to about 7 mg/mL, about 5 mg/mL to about 8 mg/mL, about 6 mg/mL to about 7 mg/mL, about 6 mg/mL to about 8 mg/mL, or about 7 mg/mL to about 8 mg/mL. In embodiments, the pegzilarginase is stored at the concentration of about 1 mg/mL, about 2 mg/mL, about 3 mg/mL, about 4 mg/mL, about 5 mg/mL, about 6 mg/mL, about 7 mg/mL, or about 8 mg/mL. In embodiments, the pegzilarginase is stored at the concentration of about 5 mg/mL.

In embodiments, the human arginase 1 is stored at the concentration of about 1 mg/mL to about 8 mg/mL. In embodiments, the human arginase 1 is stored at the concentration of at least about 1 mg/mL. In embodiments, the human arginase 1 is stored at the concentration of at most about 8 mg/mL. In embodiments, the human arginase 1 is stored at the concentration of about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 6 mg/mL, about 1 mg/mL to about 7 mg/mL, about 1 mg/mL to about 8 mg/mL, about 2 mg/mL to about 3 mg/mL, about 2 mg/mL to about 4 mg/mL, about 2 mg/mL to about 5 mg/mL, about 2 mg/mL to about 6 mg/mL, about 2 mg/mL to about 7 mg/mL, about 2 mg/mL to about 8 mg/mL, about 3 mg/mL to about 4 mg/mL, about 3 mg/mL to about 5 mg/mL, about 3 mg/mL to about 6 mg/mL, about 3 mg/mL to about 7 mg/mL, about 3 mg/mL to about 8 mg/mL, about 4 mg/mL to about 5 mg/mL, about 4 mg/mL to about 6 mg/mL, about 4 mg/mL to about 7 mg/mL, about 4 mg/mL to about 8 mg/mL, about 5 mg/mL to about 6 mg/mL, about 5 mg/mL to about 7 mg/mL, about 5 mg/mL to about 8 mg/mL, about 6 mg/mL to about 7 mg/mL, about 6 mg/mL to about 8 mg/mL, or about 7 mg/mL to about 8 mg/mL. In embodiments, the human arginase 1 is stored at the concentration of about 1 mg/mL, about 2 mg/mL, about 3 mg/mL, about 4 mg/mL, about 5 mg/mL, about 6 mg/mL, about 7 mg/mL, or about 8 mg/mL. In embodiments, the human arginase 1 is stored at the concentration of about 5 mg/mL. In embodiments, the weight of the human arginase 1 is based on the combined weight of the human arginase 1 and the polyethylene glycol conjugated with the human arginase 1.

In embodiment, the pegzilarginase is formulated in a buffer comprising 50 mM sodium chloride, 5 mM potassium phosphor, and 1.5% glycerol (w/v/) at a pH of 7.4. In embodiment, the human arginase 1 is formulated in a buffer comprising 50 mM sodium chloride, 5 mM potassium phosphor, and 1.5% glycerol (w/v/) at a pH of 7.4.

In embodiment, the pegzilarginase is a liquid. In embodiment, the pegzilarginase is lyophilized. In embodiment, the pegzilarginase is in a vial or an ampoule. In embodiment, the vial or ampoule contains about 2 mg or about 5 mg of the pegzilarginase. In embodiment, the human arginase 1 is a liquid. In embodiment, the human arginase 1 is lyophilized. In embodiment, the human arginase 1 is in a vial or an ampoule. In embodiment, the vial or ampoule contains about 2 mg or about 5 mg of the human arginase 1.

### Methods of Treatment

In one aspect, disclosed herein are methods of treating Arginase 1 (ARG1) deficiency (ARG1-D) in a subject, wherein the method comprises administering an arginase to the subject.

In embodiments, the arginase is a pegzilarginase, which is a pegylated human arginase 1 comprising a cobalt metal cofactor. In embodiments, the arginase is a human arginase 1. In embodiments, the human arginase 1 is pegylated and has a cobalt metal cofactor.

In embodiments, the pegzilarginase is administered daily, weekly, bi-weekly, monthly. In embodiments, the pegzilarginase is administered every 1, 2, 3, 4, 5, 6, 7, 8, 9, or 10 days. In embodiments, the pegzilarginase is administered weekly. In embodiments, the human arginase 1 is administered daily, weekly, bi-weekly, monthly. In embodiments, the human arginase 1 is administered weekly. In embodiments, the human arginase 1 is administered every 1, 2, 3, 4, 5, 6, 7, 8, 9, or 10 days.

In embodiments, the pegzilarginase is administered at a dose of about 0.01 mg/kg to about 0.5 mg/kg. In embodiments, the pegzilarginase is administered at a dose of about 0.05 mg/kg to about 0.2 mg/kg. In embodiments, the pegzilarginase is administered at a dose of at least about 0.01 mg/kg. In embodiments, the pegzilarginase is administered at a dose of at most about 0.5 mg/kg. In embodiments, the pegzilarginase is administered at a dose of at least about 0.05 mg/kg. In embodiments, the pegzilarginase is administered at a dose of at most about 0.2 mg/kg. In embodiments, the pegzilarginase is administered at a dose of about 0.01 mg/kg to about 0.05 mg/kg, about 0.01 mg/kg to about 0.1 mg/kg, about 0.01 mg/kg to about 0.15 mg/kg, about 0.01 mg/kg to about 0.2 mg/kg, about 0.01 mg/kg to about 0.25 mg/kg, about 0.01 mg/kg to about 0.3 mg/kg, about 0.01 mg/kg to about 0.35 mg/kg, about 0.01 mg/kg to about 0.4 mg/kg, about 0.01 mg/kg to about 0.45 mg/kg, about 0.01 mg/kg to about 0.5 mg/kg, about 0.05 mg/kg to about 0.1 mg/kg, about 0.05 mg/kg to about 0.15 mg/kg, about 0.05 mg/kg to about 0.2 mg/kg, about 0.05 mg/kg to about 0.25 mg/kg, about 0.05 mg/kg to about 0.3 mg/kg, about 0.05 mg/kg to about 0.35 mg/kg, about 0.05 mg/kg to about 0.4 mg/kg, about 0.05 mg/kg to about 0.45 mg/kg, about 0.05 mg/kg to about 0.5 mg/kg, about 0.1 mg/kg to about 0.15 mg/kg, about 0.1 mg/kg to about 0.2 mg/kg, about 0.1 mg/kg to about 0.25 mg/kg, about 0.1 mg/kg to about 0.3 mg/kg, about 0.1 mg/kg to about 0.35 mg/kg, about 0.1 mg/kg to about 0.4 mg/kg, about 0.1 mg/kg to about 0.45 mg/kg, about 0.1 mg/kg to about 0.5 mg/kg, about 0.15 mg/kg to about 0.2 mg/kg, about 0.15 mg/kg to about 0.25 mg/kg, about 0.15 mg/kg to about 0.3 mg/kg, about 0.15 mg/kg to about 0.35 mg/kg, about 0.15 mg/kg to about 0.4 mg/kg, about 0.15 mg/kg to about 0.45 mg/kg, about 0.15 mg/kg to about 0.5 mg/kg, about 0.2 mg/kg to about 0.25 mg/kg, about 0.2 mg/kg to about 0.3 mg/kg, about 0.2 mg/kg to about 0.35 mg/kg, about 0.2 mg/kg to about 0.4 mg/kg, about 0.2 mg/kg to about 0.45 mg/kg, about 0.2 mg/kg to about 0.5 mg/kg, about 0.25 mg/kg to about 0.3 mg/kg, about 0.25 mg/kg to about 0.35 mg/kg, about 0.25 mg/kg to about 0.4 mg/kg, about 0.25 mg/kg to about 0.45 mg/kg, about 0.25 mg/kg to about 0.5 mg/kg, about 0.3 mg/kg to about 0.35 mg/kg, about 0.3 mg/kg to about 0.4 mg/kg, about 0.3 mg/kg to about 0.45 mg/kg, about 0.3 mg/kg to about 0.5 mg/kg, about 0.35 mg/kg to about 0.4 mg/kg, about 0.35 mg/kg to about 0.45 mg/kg, about 0.35 mg/kg to about 0.5 mg/kg, about 0.4 mg/kg to about 0.45 mg/kg, about 0.4 mg/kg to about 0.5 mg/kg, or about 0.45 mg/kg to about 0.5 mg/kg. In embodiments, the pegzilarginase is administered at a dose of about 0.01 mg/kg, about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, or about 0.5 mg/kg.

In embodiments, the human arginase 1 is administered at a dose of about 0.01 mg/kg to about 0.5 mg/kg. In embodiments, the human arginase 1 is administered at a dose of about 0.05 mg/kg to about 0.2 mg/kg. In embodiments, the human arginase 1 is administered at a dose of at least about 0.01 mg/kg. In embodiments, the human arginase 1 is administered at a dose of at most about 0.5 mg/kg. In embodiments, the human arginase 1 is administered at a dose of at least about 0.05 mg/kg. In embodiments, the human arginase 1 is administered at a dose of at most about 0.2 mg/kg. In embodiments, the human arginase 1 is administered at a dose of about 0.01 mg/kg to about 0.05 mg/kg, about 0.01 mg/kg to about 0.1 mg/kg, about 0.01 mg/kg to about 0.15 mg/kg, about 0.01 mg/kg to about 0.2 mg/kg, about 0.01 mg/kg to about 0.25 mg/kg, about 0.01 mg/kg to about 0.3 mg/kg, about 0.01 mg/kg to about 0.35 mg/kg, about 0.01 mg/kg to about 0.4 mg/kg, about 0.01 mg/kg to about 0.45 mg/kg, about 0.01 mg/kg to about 0.5 mg/kg, about 0.05 mg/kg to about 0.1 mg/kg, about 0.05 mg/kg to about 0.15 mg/kg, about 0.05 mg/kg to about 0.2 mg/kg, about 0.05 mg/kg to about 0.25 mg/kg, about 0.05 mg/kg to about 0.3 mg/kg, about 0.05 mg/kg to about 0.35 mg/kg, about 0.05 mg/kg to about 0.4 mg/kg, about 0.05 mg/kg to about 0.45 mg/kg, about 0.05 mg/kg to about 0.5 mg/kg, about 0.1 mg/kg to about 0.15 mg/kg, about 0.1 mg/kg to about 0.2 mg/kg, about 0.1 mg/kg to about 0.25 mg/kg, about 0.1 mg/kg to about 0.3 mg/kg, about 0.1 mg/kg to about 0.35 mg/kg, about 0.1 mg/kg to about 0.4 mg/kg, about 0.1 mg/kg to about 0.45 mg/kg, about 0.1 mg/kg to about 0.5 mg/kg, about 0.15 mg/kg to about 0.2 mg/kg, about 0.15 mg/kg to about 0.25 mg/kg, about 0.15 mg/kg to about 0.3 mg/kg, about 0.15 mg/kg to about 0.35 mg/kg, about 0.15 mg/kg to about 0.4 mg/kg, about 0.15 mg/kg to about 0.45 mg/kg, about 0.15 mg/kg to about 0.5 mg/kg, about 0.2 mg/kg to about 0.25 mg/kg, about 0.2 mg/kg to about 0.3 mg/kg, about 0.2 mg/kg to about 0.35 mg/kg, about 0.2 mg/kg to about 0.4 mg/kg, about 0.2 mg/kg to about 0.45 mg/kg, about 0.2 mg/kg to about 0.5 mg/kg, about 0.25 mg/kg to about 0.3 mg/kg, about 0.25 mg/kg to about 0.35 mg/kg, about 0.25 mg/kg to about 0.4 mg/kg, about 0.25 mg/kg to about 0.45 mg/kg, about 0.25 mg/kg to about 0.5 mg/kg, about 0.3 mg/kg to about 0.35 mg/kg, about 0.3 mg/kg to about 0.4 mg/kg, about 0.3 mg/kg to about 0.45 mg/kg, about 0.3 mg/kg to about 0.5 mg/kg, about 0.35 mg/kg to about 0.4 mg/kg, about 0.35 mg/kg to about 0.45 mg/kg, about 0.35 mg/kg to about 0.5 mg/kg, about 0.4 mg/kg to about 0.45 mg/kg, about 0.4 mg/kg to about 0.5 mg/kg, or about 0.45 mg/kg to about 0.5 mg/kg. In embodiments, the human arginase 1 is administered at a dose of about 0.01 mg/kg, about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, or about 0.5 mg/kg. In embodiments, the weight of the human arginase 1 is based on the combined weight of the human arginase 1 and the polyethylene glycol conjugated with the human arginase 1.

In embodiments, the initial dose of the pegzilarginase is about 0.01 mg/kg to about 0.2 mg/kg. In embodiments, the initial dose of the pegzilarginase is at least about 0.01 mg/kg. In embodiments, the initial dose of the pegzilarginase is at most about 0.2 mg/kg. In embodiments, the initial dose of the pegzilarginase is about 0.01 mg/kg to about 0.03 mg/kg, about 0.01 mg/kg to about 0.05 mg/kg, about 0.01 mg/kg to about 0.07 mg/kg, about 0.01 mg/kg to about 0.09 mg/kg, about 0.01 mg/kg to about 0.1 mg/kg, about 0.01 mg/kg to about 0.12 mg/kg, about 0.01 mg/kg to about 0.14 mg/kg, about 0.01 mg/kg to about 0.16 mg/kg, about 0.01 mg/kg to about 0.18 mg/kg, about 0.01 mg/kg to about 0.2 mg/kg, about 0.03 mg/kg to about 0.05 mg/kg, about 0.03 mg/kg to about 0.07 mg/kg, about 0.03 mg/kg to about 0.09 mg/kg, about 0.03 mg/kg to about 0.1 mg/kg, about 0.03 mg/kg to about 0.12 mg/kg, about 0.03 mg/kg to about 0.14 mg/kg, about 0.03 mg/kg to about 0.16 mg/kg, about 0.03 mg/kg to about 0.18 mg/kg, about 0.03 mg/kg to about 0.2 mg/kg, about 0.05 mg/kg to about 0.07 mg/kg, about 0.05 mg/kg to about 0.09 mg/kg, about 0.05 mg/kg to about 0.1 mg/kg, about 0.05 mg/kg to about 0.12 mg/kg, about 0.05 mg/kg to about 0.14 mg/kg, about 0.05 mg/kg to about 0.16 mg/kg, about 0.05 mg/kg to about 0.18 mg/kg, about 0.05 mg/kg to about 0.2 mg/kg, about 0.07 mg/kg to about 0.09 mg/kg, about 0.07 mg/kg to about 0.1 mg/kg, about 0.07 mg/kg to about 0.12 mg/kg, about 0.07 mg/kg to about 0.14 mg/kg, about 0.07 mg/kg to about 0.16 mg/kg, about 0.07 mg/kg to about 0.18 mg/kg, about 0.07 mg/kg to about 0.2 mg/kg, about 0.09 mg/kg to about 0.1 mg/kg, about 0.09 mg/kg to about 0.12 mg/kg, about 0.09 mg/kg to about 0.14 mg/kg, about 0.09 mg/kg to about 0.16 mg/kg, about 0.09 mg/kg to about 0.18 mg/kg, about 0.09 mg/kg to about 0.2 mg/kg, about 0.1 mg/kg to about 0.12 mg/kg, about 0.1 mg/kg to about 0.14 mg/kg, about 0.1 mg/kg to about 0.16 mg/kg, about 0.1 mg/kg to about 0.18 mg/kg, about 0.1 mg/kg to about 0.2 mg/kg, about 0.12 mg/kg to about 0.14 mg/kg, about 0.12 mg/kg to about 0.16 mg/kg, about 0.12 mg/kg to about 0.18 mg/kg, about 0.12 mg/kg to about 0.2 mg/kg, about 0.14 mg/kg to about 0.16 mg/kg, about 0.14 mg/kg to about 0.18 mg/kg, about 0.14 mg/kg to about 0.2 mg/kg, about 0.16 mg/kg to about 0.18 mg/kg, about 0.16 mg/kg to about 0.2 mg/kg, or about 0.18 mg/kg to about 0.2 mg/kg. In embodiments, the initial dose of the pegzilarginase is about 0.01 mg/kg, about 0.03 mg/kg, about 0.05 mg/kg, about 0.07 mg/kg, about 0.09 mg/kg, about 0.1 mg/kg, about 0.12 mg/kg, about 0.14 mg/kg, about 0.16 mg/kg, about 0.18 mg/kg, or about 0.2 mg/kg. In embodiments, the initial dose of the pegzilarginase is about 0.1 mg/kg.

In embodiments, the initial dose of the human arginase 1 is about 0.01 mg/kg to about 0.2 mg/kg. In embodiments, the initial dose of the human arginase 1 is at least about 0.01 mg/kg. In embodiments, the initial dose of the human arginase 1 is at most about 0.2 mg/kg. In embodiments, the initial dose of the human arginase 1 is about 0.01 mg/kg to about 0.03 mg/kg, about 0.01 mg/kg to about 0.05 mg/kg, about 0.01 mg/kg to about 0.07 mg/kg, about 0.01 mg/kg to about 0.09 mg/kg, about 0.01 mg/kg to about 0.1 mg/kg, about 0.01 mg/kg to about 0.12 mg/kg, about 0.01 mg/kg to about 0.14 mg/kg, about 0.01 mg/kg to about 0.16 mg/kg, about 0.01 mg/kg to about 0.18 mg/kg, about 0.01 mg/kg to about 0.2 mg/kg, about 0.03 mg/kg to about 0.05 mg/kg, about 0.03 mg/kg to about 0.07 mg/kg, about 0.03 mg/kg to about 0.09 mg/kg, about 0.03 mg/kg to about 0.1 mg/kg, about 0.03 mg/kg to about 0.12 mg/kg, about 0.03 mg/kg to about 0.14 mg/kg, about 0.03 mg/kg to about 0.16 mg/kg, about 0.03 mg/kg to about 0.18 mg/kg, about 0.03 mg/kg to about 0.2 mg/kg, about 0.05 mg/kg to about 0.07 mg/kg, about 0.05 mg/kg to about 0.09 mg/kg, about 0.05 mg/kg to about 0.1 mg/kg, about 0.05 mg/kg to about 0.12 mg/kg, about 0.05 mg/kg to about 0.14 mg/kg, about 0.05 mg/kg to about 0.16 mg/kg, about 0.05 mg/kg to about 0.18 mg/kg, about 0.05 mg/kg to about 0.2 mg/kg, about 0.07 mg/kg to about 0.09 mg/kg, about 0.07 mg/kg to about 0.1 mg/kg, about 0.07 mg/kg to about 0.12 mg/kg, about 0.07 mg/kg to about 0.14 mg/kg, about 0.07 mg/kg to about 0.16 mg/kg, about 0.07 mg/kg to about 0.18 mg/kg, about 0.07 mg/kg to about 0.2 mg/kg, about 0.09 mg/kg to about 0.1 mg/kg, about 0.09 mg/kg to about 0.12 mg/kg, about 0.09 mg/kg to about 0.14 mg/kg, about 0.09 mg/kg to about 0.16 mg/kg, about 0.09 mg/kg to about 0.18 mg/kg, about 0.09 mg/kg to about 0.2 mg/kg, about 0.1 mg/kg to about 0.12 mg/kg, about 0.1 mg/kg to about 0.14 mg/kg, about 0.1 mg/kg to about 0.16 mg/kg, about 0.1 mg/kg to about 0.18 mg/kg, about 0.1 mg/kg to about 0.2 mg/kg, about 0.12 mg/kg to about 0.14 mg/kg, about 0.12 mg/kg to about 0.16 mg/kg, about 0.12 mg/kg to about 0.18 mg/kg, about 0.12 mg/kg to about 0.2 mg/kg, about 0.14 mg/kg to about 0.16 mg/kg, about 0.14 mg/kg to about 0.18 mg/kg, about 0.14 mg/kg to about 0.2 mg/kg, about 0.16 mg/kg to about 0.18 mg/kg, about 0.16 mg/kg to about 0.2 mg/kg, or about 0.18 mg/kg to about 0.2 mg/kg. In embodiments, the initial dose of the human arginase 1 is about 0.01 mg/kg, about 0.03 mg/kg, about 0.05 mg/kg, about 0.07 mg/kg, about 0.09 mg/kg, about 0.1 mg/kg, about 0.12 mg/kg, about 0.14 mg/kg, about 0.16 mg/kg, about 0.18 mg/kg, or about 0.2 mg/kg. In embodiments, the initial dose of the human arginase 1 is about 0.1 mg/kg.

In embodiments, subsequent doses of the pegzilarginase are adjusted based on the subject's plasma arginine level prior to the administration of the subsequent doses. In embodiments, the subsequent doses of the pegzilarginase are increased if the subject's plasma arginine level prior to the administration of the subsequent doses is higher than about 100 μM. In embodiments, the subsequent doses of the pegzilarginase are increased if the subject's plasma arginine level prior to the administration of the subsequent doses is higher than about 115 μM. In embodiments, the subsequent doses of the pegzilarginase are increased if the subject's plasma arginine level prior to the administration of the subsequent doses is higher than about 130 μM. In embodiments, the subsequent doses of the pegzilarginase are increased if the subject's plasma arginine level prior to the administration of the subsequent doses is higher than about 135 μM. In embodiments, the subsequent doses of the pegzilarginase are increased if the subject's plasma arginine level prior to the administration of the subsequent doses is higher than about 150 μM. In embodiments, the subsequent doses of the pegzilarginase are increased if the subject's plasma arginine level prior to the administration of the subsequent doses is higher than about 165 μM. In embodiments, the subsequent doses of the pegzilarginase are increased if the subject's plasma arginine level prior to the administration of the subsequent doses is higher than about 180 μM. In embodiments, the subsequent doses of the pegzilarginase are increased if the subject's plasma arginine level prior to the administration of the subsequent doses is higher than about 195 μM. In embodiments, the subsequent doses of the pegzilarginase are increased if the subject's plasma arginine level prior to the administration of the subsequent doses is higher than about 200 μM. In embodiments, the subsequent doses of the pegzilarginase are decreased if the subject's plasma arginine level prior to the administration of the subsequent doses is lower than about 50 μM. In embodiments, the subsequent doses of the pegzilarginase are decreased if the subject's plasma arginine level prior to the administration of the subsequent doses is lower than about 40 M. In embodiments, the subsequent doses of the pegzilarginase are decreased if the subject's plasma arginine level prior to the administration of the subsequent doses is lower than about 30 μM. In embodiments, the subsequent doses of the pegzilarginase are decreased if the subject's plasma arginine level prior to the administration of the subsequent doses is lower than about 20 μM.

In embodiments, subsequent doses of the human arginase 1 are adjusted based on the subject's plasma arginine level prior to the administration of the subsequent doses. In embodiments, the subsequent doses of the human arginase 1 are increased if the subject's plasma arginine level prior to the administration of the subsequent doses is higher than about 100 μM. In embodiments, the subsequent doses of the human arginase 1 are increased if the subject's plasma arginine level prior to the administration of the subsequent doses is higher than about 115 μM. In embodiments, the subsequent doses of the human arginase 1 are increased if the subject's plasma arginine level prior to the administration of the subsequent doses is higher than about 130 μM. In embodiments, the subsequent doses of the human arginase 1 are increased if the subject's plasma arginine level prior to the administration of the subsequent doses is higher than about 135 μM. In embodiments, the subsequent doses of the human arginase 1 are increased if the subject's plasma arginine level prior to the administration of the subsequent doses is higher than about 150 μM. In embodiments, the subsequent doses of the human arginase 1 are increased if the subject's plasma arginine level prior to the administration of the subsequent doses is higher than about 165 μM. In embodiments, the subsequent doses of the human arginase 1 are increased if the subject's plasma arginine level prior to the administration of the subsequent doses is higher than about 180 μM. In embodiments, the subsequent doses of the human arginase 1 are increased if the subject's plasma arginine level prior to the administration of the subsequent doses is higher than about 195 μM. In embodiments, the subsequent doses of the human arginase 1 are increased if the subject's plasma arginine level prior to the administration of the subsequent doses is higher than about 200 μM. In embodiments, the subsequent doses of the human arginase 1 are decreased if the subject's plasma arginine level prior to the administration of the subsequent doses is lower than about 50 μM. In embodiments, the subsequent doses of the human arginase 1 are decreased if the subject's plasma arginine level prior to the administration of the subsequent doses is lower than about 40 μM. In embodiments, the subsequent doses of the human arginase 1 are decreased if the subject's plasma arginine level prior to the administration of the subsequent doses is lower than about 30 μM. In embodiments, the subsequent doses of the human arginase 1 are decreased if the subject's plasma arginine level prior to the administration of the subsequent doses is lower than about 20 μM.

In embodiments, the subsequent doses of the pegzilarginase are adjusted between 0.01 mg/kg and 0.1 mg/kg each time based on the subject's plasma arginine level resulting from the prior dose. In embodiments, the subsequent doses of the pegzilarginase are adjusted at least about 0.01 mg/kg each time based on the subject's plasma arginine level resulting from the prior dose. In embodiments, the subsequent doses of the pegzilarginase are adjusted at most about 0.2 mg/kg each time based on the subject's plasma arginine level resulting from the prior dose. In embodiments, the subsequent doses of the pegzilarginase are adjusted about 0.01 mg/kg to about 0.03 mg/kg, about 0.01 mg/kg to about 0.05 mg/kg, about 0.01 mg/kg to about 0.07 mg/kg, about 0.01 mg/kg to about 0.1 mg/kg, about 0.01 mg/kg to about 0.2 mg/kg, about 0.03 mg/kg to about 0.05 mg/kg, about 0.03 mg/kg to about 0.07 mg/kg, about 0.03 mg/kg to about 0.1 mg/kg, about 0.03 mg/kg to about 0.2 mg/kg, about 0.05 mg/kg to about 0.07 mg/kg, about 0.05 mg/kg to about 0.1 mg/kg, about 0.05 mg/kg to about 0.2 mg/kg, about 0.07 mg/kg to about 0.1 mg/kg, about 0.07 mg/kg to about 0.2 mg/kg, or about 0.1 mg/kg to about 0.2 mg/kg each time based on the subject's plasma arginine level resulting from the prior dose. In embodiments, the subsequent doses of the pegzilarginase are adjusted about 0.01 mg/kg, about 0.03 mg/kg, about 0.05 mg/kg, about 0.07 mg/kg, about 0.1 mg/kg, or about 0.2 mg/kg each time based on the subject's plasma arginine level resulting from the prior dose. In embodiments, the subsequent doses of the pegzilarginase are adjusted about 0.05 mg/kg each time based on the subject's plasma arginine level resulting from the prior dose.

In embodiments, the subsequent doses of the human arginase 1 are adjusted between 0.01 mg/kg and 0.1 mg/kg each time based on the subject's plasma arginine level resulting from the prior dose. In embodiments, the subsequent doses of the human arginase 1 are adjusted at least about 0.01 mg/kg each time based on the subject's plasma arginine level resulting from the prior dose. In embodiments, the subsequent doses of the human arginase 1 are adjusted at most about 0.2 mg/kg each time based on the subject's plasma arginine level resulting from the prior dose. In embodiments, the subsequent doses of the human arginase 1 are adjusted about 0.01 mg/kg to about 0.03 mg/kg, about 0.01 mg/kg to about 0.05 mg/kg, about 0.01 mg/kg to about 0.07 mg/kg, about 0.01 mg/kg to about 0.1 mg/kg, about 0.01 mg/kg to about 0.2 mg/kg, about 0.03 mg/kg to about 0.05 mg/kg, about 0.03 mg/kg to about 0.07 mg/kg, about 0.03 mg/kg to about 0.1 mg/kg, about 0.03 mg/kg to about 0.2 mg/kg, about 0.05 mg/kg to about 0.07 mg/kg, about 0.05 mg/kg to about 0.1 mg/kg, about 0.05 mg/kg to about 0.2 mg/kg, about 0.07 mg/kg to about 0.1 mg/kg, about 0.07 mg/kg to about 0.2 mg/kg, or about 0.1 mg/kg to about 0.2 mg/kg each time based on the subject's plasma arginine level resulting from the prior dose. In embodiments, the subsequent doses of the human arginase 1 are adjusted about 0.01 mg/kg, about 0.03 mg/kg, about 0.05 mg/kg, about 0.07 mg/kg, about 0.1 mg/kg, or about 0.2 mg/kg each time based on the subject's plasma arginine level resulting from the prior dose. In embodiments, the subsequent doses of the human arginase 1 are adjusted about 0.05 mg/kg each time based on the subject's plasma arginine level resulting from the prior dose.

In embodiments, the subject does not receive pre-medication before the initial dose of the pegzilarginase. In embodiments, the subject receives pre-medication before the initial dose of the pegzilarginase. In embodiments, the subject does not receive pre-medication before the initial dose of the human arginase 1. In embodiments, the subject receives pre-medication before the initial dose of the human arginase 1.

In embodiments, the subject's plasma arginine level is at least 200 UM before the initial dose of the pegzilarginase. In embodiments, the subject's plasma arginine level is at least 210 UM before the initial dose of the pegzilarginase. In embodiments, the subject's plasma arginine level is at least 230 μM before the initial dose of the pegzilarginase. In embodiments, the subject's plasma arginine level is at least 250 μM before the initial dose of the pegzilarginase. In embodiments, the subject's plasma arginine level is at least 270 μM before the initial dose of the pegzilarginase. In embodiments, the subject's plasma arginine level is at least 290 μM before the initial dose of the pegzilarginase. In embodiments, the subject's plasma arginine level is at least 310 μM before the initial dose of the pegzilarginase.

In embodiments, the subject's plasma arginine level is at least 200 μM before the initial dose of the human arginase 1. In embodiments, the subject's plasma arginine level is at least 210 μM before the initial dose of the human arginase 1. In embodiments, the subject's plasma arginine level is at least 230 UM before the initial dose of the human arginase 1. In embodiments, the subject's plasma arginine level is at least 250 UM before the initial dose of the human arginase 1. In embodiments, the subject's plasma arginine level is at least 270 UM before the initial dose of the human arginase 1. In embodiments, the subject's plasma arginine level is at least 290 μM before the initial dose of the human arginase 1. In embodiments, the subject's plasma arginine level is at least 310 μM before the initial dose of the human arginase 1.

In embodiments, the subject is an adult. In embodiments, the subject is less than 18 years old. In embodiments, the subject is at least 2 years old.

In embodiments, the subject is classified as GMFCS Level II. In embodiments, the subject is classified as GMFCS Level III. In embodiments, the subject is classified as GMFCS Level IV. In embodiments, the subject is classified as GMFCS Level V.

A plasma level of arginine and/or a guanidino compound may be reduced to a normal range or a normal level, which may mean that at some point subsequent to administration of an initial dose and/or a repeat dose of an arginase, the plasma level of arginine and/or one or more guanidino compounds has a value within the range of 40 μM to 115 μM for arginine as described by Luneburg, N. et al., (2011). For example, a patient's plasma levels for arginine and/or one or more of the guanidino compounds may oscillate in and out of the normal range during treatment with an arginase as disclosed herein. This patient is considered to have a plasma level of the assayed arginine and/or guanidino compounds that is reduced to the normal level or in the normal range. As another example, a patient may have an average amount of plasma levels of arginine and/or one or more guanidino compounds that is within the normal range (e.g., 40 μM to 115 μM for arginine as described by Luneburg, N. et al., (2011)), subsequent to receiving one or more dosages of the arginase as disclosed herein. Thus, the disclosed compositions and methods herein may reduce a subject's plasma level of arginine and/or a guanidino compound to a normal level or to the normal range at least one time after receiving an initial dosage and/or a repeat dosage of the arginase. In some patients, it was observed that plasma levels of arginine and/or a guanidino compound could be maintained, on average, at normal levels or within the normal range according to the disclosed methods herein.

In embodiments, the subject's plasma arginine level is between about 40 μM and about 115 μM after the treatment. In embodiments, the subject's plasma arginine level is between about 40 μM and about 115 μM after 2 doses of the pegzilarginase. In embodiments, the subject's plasma arginine level is between about 40 M and about 115 μM after 4 doses of the pegzilarginase. In embodiments, the subject's plasma arginine level is between about 40 μM and about 115 μM after 6 doses of the pegzilarginase. In embodiments, the subject's plasma arginine level is between about 40 μM and about 115 M after 8 doses of the pegzilarginase. In embodiments, the subject's plasma arginine level is between about 40 μM and about 115 μM after 10 doses of the pegzilarginase. In embodiments, the subject's plasma arginine level is between about 40 μM and about 115 μM after 12 doses of the pegzilarginase. In embodiments, the subject's plasma arginine level is between about 40 μM and about 115 μM after 14 doses of the pegzilarginase. In embodiments, the subject's plasma arginine level is between about 40 μM and about 115 μM after 16 doses of the pegzilarginase. In embodiments, the subject's plasma arginine level is between about 40 μM and about 115 μM after 18 doses of the pegzilarginase. In embodiments, the subject's plasma arginine level is between about 40 μM and about 115 μM after 20 doses of the pegzilarginase. In embodiments, the subject's plasma arginine level is between about 40 μM and about 115 μM after 22 doses of the pegzilarginase. In embodiments, the subject's plasma arginine level is between about 40 μM and about 115 μM after 24 doses of the pegzilarginase.

In embodiments, the subject's plasma arginine level is between about 40 μM and about 115 M after the treatment. In embodiments, the subject's plasma arginine level is between about 40 μM and about 115 μM after 2 doses of the human arginase 1. In embodiments, the subject's plasma arginine level is between about 40 μM and about 115 μM after 4 doses of the human arginase 1. In embodiments, the subject's plasma arginine level is between about 40 μM and about 115 μM after 6 doses of the human arginase 1. In embodiments, the subject's plasma arginine level is between about 40 μM and about 115 μM after 8 doses of the human arginase 1. In embodiments, the subject's plasma arginine level is between about 40 μM and about 115 μM after 10 doses of the human arginase 1. In embodiments, the subject's plasma arginine level is between about 40 μM and about 115 μM after 12 doses of the human arginase 1. In embodiments, the subject's plasma arginine level is between about 40 μM and about 115 μM after 14 doses of the human arginase 1. In embodiments, the subject's plasma arginine level is between about 40 μM and about 115 μM after 16 doses of the human arginase 1. In embodiments, the subject's plasma arginine level is between about 40 μM and about 115 μM after 18 doses of the human arginase 1. In embodiments, the subject's plasma arginine level is between about 40 μM and about 115 μM after 20 doses of the human arginase 1. In embodiments, the subject's plasma arginine level is between about 40 M and about 115 μM after 22 doses of the human arginase 1. In embodiments, the subject's plasma arginine level is between about 40 μM and about 115 μM after 24 doses of the human arginase 1.

In embodiments, the subject's intake of natural protein is increased after one or more doses of the pegzilarginase. the subject's intake of essential amino acid mixture is reduced after one or more doses of the pegzilarginase. In embodiments, the subject's relative intake of essential amino acid mixture compared to natural protein is reduced after one or more doses of the pegzilarginase. In embodiments, after one or more doses of the pegzilarginase, the primary source of the subject's protein intake is changed from an arginine-free (or low arginine) essential amino acid mixture to the addition of any food item that contains protein. In embodiments, after one or more doses of the pegzilarginase, the primary source of the subject's protein intake is changed from an arginine-free (or low arginine) essential amino acid mixture to natural protein.

In embodiments, the subject's intake of total protein is greater than 30 g/day after one or more doses of the pegzilarginase. In embodiments, the subject's intake of total protein is greater than 35 g/day after one or more doses of the pegzilarginase. In embodiments, the subject's intake of total protein is greater than 40 g/day after one or more doses of the pegzilarginase. In embodiments, the subject's intake of total protein is greater than 45 g/day after one or more doses of the pegzilarginase. In embodiments, the subject's intake of total protein is greater than 50 g/day after one or more doses of the pegzilarginase. In embodiments, the subject's intake of total protein increases by at least 5% after one or more doses of the pegzilarginase. In embodiments, the subject's intake of total protein increases by at least 10% after one or more doses of the pegzilarginase. In embodiments, the subject's intake of total protein increases by at least 15% after one or more doses of the pegzilarginase. In embodiments, the subject's intake of total protein increases by at least 20% after one or more doses of the pegzilarginase. In embodiments, the subject's intake of total protein increases by at least 25% after one or more doses of the pegzilarginase. In embodiments, the subject's intake of natural protein is increased to at least 45% of the intake of total protein after one or more doses of the pegzilarginase. In embodiments, the subject's intake of natural protein is increased to at least 50% of the intake of total protein after one or more doses of the pegzilarginase. In embodiments, the subject's intake of natural protein is increased to at least 55% of the intake of total protein after one or more doses of the pegzilarginase. In embodiments, the subject's intake of natural protein is increased to at least 60% of the intake of total protein after one or more doses of the pegzilarginase. In embodiments, the subject's intake of natural protein is increased to at least 65% of the intake of total protein after one or more doses of the pegzilarginase. In embodiments, the subject's intake of natural protein is increased to at least 70% of the intake of total protein after one or more doses of the pegzilarginase.

In embodiments, the subject's intake of natural protein is increased after one or more doses of the human arginase 1. the subject's intake of essential amino acid mixture is reduced after one or more doses of the human arginase 1. In embodiments, the subject's relative intake of essential amino acid mixture compared to natural protein is reduced after one or more doses of the human arginase 1. In embodiments, after one or more doses of the human arginase 1, the primary source of the subject's protein intake is changed from an arginine-free (or low arginine) essential amino acid mixture to the addition of any food item that contains protein. In embodiments, after one or more doses of the human arginase 1, the primary source of the subject's protein intake is changed from an arginine-free (or low arginine) essential amino acid mixture to natural protein.

In embodiments, the subject's intake of total protein is greater than 30 g/day after one or more doses of the human arginase 1. In embodiments, the subject's intake of total protein is greater than 35 g/day after one or more doses of the human arginase 1. In embodiments, the subject's intake of total protein is greater than 40 g/day after one or more doses of the human arginase 1. In embodiments, the subject's intake of total protein is greater than 45 g/day after one or more doses of the human arginase 1. In embodiments, the subject's intake of total protein is greater than 50 g/day after one or more doses of the human arginase 1. In embodiments, the subject's intake of total protein increases by at least 5% after one or more doses of the human arginase 1. In embodiments, the subject's intake of total protein increases by at least 10% after one or more doses of the human arginase 1. In embodiments, the subject's intake of total protein increases by at least 15% after one or more doses of the human arginase 1. In embodiments, the subject's intake of total protein increases by at least 20% after one or more doses of the human arginase 1. In embodiments, the subject's intake of total protein increases by at least 25% after one or more doses of the human arginase 1. In embodiments, the subject's intake of natural protein is increased to at least 45% of the intake of total protein after one or more doses of the human arginase 1. In embodiments, the subject's intake of natural protein is increased to at least 50% of the intake of total protein after one or more doses of the human arginase 1. In embodiments, the subject's intake of natural protein is increased to at least 55% of the intake of total protein after one or more doses of the human arginase 1. In embodiments, the subject's intake of natural protein is increased to at least 60% of the intake of total protein after one or more doses of the human arginase 1. In embodiments, the subject's intake of natural protein is increased to at least 65% of the intake of total protein after one or more doses of the human arginase 1. In embodiments, the subject's intake of natural protein is increased to at least 70% of the intake of total protein after one or more doses of the human arginase 1.

In embodiments, the pegzilarginase or the human arginase 1 or compositions comprising the same is used for diet liberalization for arginase 1 deficiency patients. In embodiments, the pegzilarginase or the human arginase 1 or compositions comprising the same is used for treating protein aversion in a patient with a urea cycle disorder. In embodiments, the pegzilarginase or the human arginase 1 or compositions comprising the same is used for treating diseases or conditions related to ARG1-D. The related diseases or conditions can be, but are not limited to, GAMT, S-adenosylhomocysteine hydrolase deficiency, CNS injury, and renal damage.

In embodiments, the subject does not have a hyperammonemic episode. In embodiments, the subject does not have an active infection requiring anti-infective therapy. In embodiments, the subject does not have an active infection with human immunodeficiency virus, hepatitis B, or hepatitis C. In embodiments, the subject does not have extreme mobility deficit, defined as either the inability to be assessed on the Gillette Functional Assessment Questionnaire (GFAQ) or a score of 1 on the GFAQ. In embodiments, the subject does not have a history of hypersensitivity to polyethylene glycol (PEG). In embodiments, the subject has not been treated with botulinum toxin. In embodiments, the subject does not have a liver or hematopoietic transplant.

In embodiments, the subject's mobility is improved after one or more doses of the pegzilarginase. In embodiments, arginine metabolite accumulation is reduced after one or more doses of the pegzilarginase. In embodiments, the subject's mobility is improved after one or more doses of the human arginase 1. In embodiments, arginine metabolite accumulation is reduced after one or more doses of the human arginase 1. In embodiments, accumulated guanidino compounds in the blood and cerebrospinal fluid of the subject may be reduced. In embodiments, the guanidino compounds are selected from the group consisting of Alpha-N-Acetylarginine, Alpha-keto-d-Guanidinovaleric Acid, Arginic Acid, and Guanidinoacetic Acid.

In embodiments, the pegzilarginase is administered at the concentration of about 1 mg/mL to about 8 mg/mL. In embodiments, the pegzilarginase is administered at the concentration of at least about 1 mg/mL. In embodiments, the pegzilarginase is administered at the concentration of at most about 8 mg/mL. In embodiments, the pegzilarginase is administered at the concentration of about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 6 mg/mL, about 1 mg/mL to about 7 mg/mL, about 1 mg/mL to about 8 mg/mL, about 2 mg/mL to about 3 mg/mL, about 2 mg/mL to about 4 mg/mL, about 2 mg/mL to about 5 mg/mL, about 2 mg/mL to about 6 mg/mL, about 2 mg/mL to about 7 mg/mL, about 2 mg/mL to about 8 mg/mL, about 3 mg/mL to about 4 mg/mL, about 3 mg/mL to about 5 mg/mL, about 3 mg/mL to about 6 mg/mL, about 3 mg/mL to about 7 mg/mL, about 3 mg/mL to about 8 mg/mL, about 4 mg/mL to about 5 mg/mL, about 4 mg/mL to about 6 mg/mL, about 4 mg/mL to about 7 mg/mL, about 4 mg/mL to about 8 mg/mL, about 5 mg/mL to about 6 mg/mL, about 5 mg/mL to about 7 mg/mL, about 5 mg/mL to about 8 mg/mL, about 6 mg/mL to about 7 mg/mL, about 6 mg/mL to about 8 mg/mL, or about 7 mg/mL to about 8 mg/mL. In embodiments, the pegzilarginase is administered at the concentration of about 1 mg/mL, about 2 mg/mL, about 3 mg/mL, about 4 mg/mL, about 5 mg/mL, about 6 mg/mL, about 7 mg/mL, or about 8 mg/mL. In embodiments, the pegzilarginase is administered at the concentration of about 5 mg/mL.

In embodiments, the human arginase 1 is administered at the concentration of about 1 mg/mL to about 8 mg/mL. In embodiments, the human arginase 1 is administered at the concentration of at least about 1 mg/mL. In embodiments, the human arginase 1 is administered at the concentration of at most about 8 mg/mL. In embodiments, the human arginase 1 is administered at the concentration of about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 6 mg/mL, about 1 mg/mL to about 7 mg/mL, about 1 mg/mL to about 8 mg/mL, about 2 mg/mL to about 3 mg/mL, about 2 mg/mL to about 4 mg/mL, about 2 mg/mL to about 5 mg/mL, about 2 mg/mL to about 6 mg/mL, about 2 mg/mL to about 7 mg/mL, about 2 mg/mL to about 8 mg/mL, about 3 mg/mL to about 4 mg/mL, about 3 mg/mL to about 5 mg/mL, about 3 mg/mL to about 6 mg/mL, about 3 mg/mL to about 7 mg/mL, about 3 mg/mL to about 8 mg/mL, about 4 mg/mL to about 5 mg/mL, about 4 mg/mL to about 6 mg/mL, about 4 mg/mL to about 7 mg/mL, about 4 mg/mL to about 8 mg/mL, about 5 mg/mL to about 6 mg/mL, about 5 mg/mL to about 7 mg/mL, about 5 mg/mL to about 8 mg/mL, about 6 mg/mL to about 7 mg/mL, about 6 mg/mL to about 8 mg/mL, or about 7 mg/mL to about 8 mg/mL. In embodiments, the human arginase 1 is administered at the concentration of about 1 mg/mL, about 2 mg/mL, about 3 mg/mL, about 4 mg/mL, about 5 mg/mL, about 6 mg/mL, about 7 mg/mL, or about 8 mg/mL. In embodiments, the human arginase 1 is administered at the concentration of about 5 mg/mL. In embodiments, the weight of the human arginase 1 is based on the combined weight of the human arginase 1 and the polyethylene glycol conjugated with the human arginase 1.

### EMBODIMENTS

In one set of embodiments (embodiment set A), provided are:
A1. A method of treating Arginase 1 (ARG1) deficiency (ARG1-D) in a subject, the method comprising administering a pegzilarginase to the subject,
1) wherein the pegzilarginase is a pegylated human arginase 1 comprising a cobalt metal cofactor, and
2) wherein the pegzilarginase is administered weekly at a dose of from about 0.05 mg/kg to about 0.2 mg/kg.
A2. The method of embodiment A1, wherein the subject's plasma arginine level is between about 40 μM and about 115 μM after 12 doses of the pegzilarginase.
A3. The method of embodiment A1 or A2, wherein the initial dose of the pegzilarginase is about 0.1 mg/kg.
A4. The method of any one of embodiments A1-A3, wherein subsequent doses of the pegzilarginase are adjusted based on the subject's plasma arginine level prior to the administration of the subsequent doses.
A5. The method of embodiment A4, wherein the subsequent doses of the pegzilarginase are increased if the subject's plasma arginine level prior to the administration of the subsequent doses is higher than about 115 μM, optionally higher than about 150 μM.
A6. The method of embodiment A4, wherein the subsequent doses of the pegzilarginase are decreased if the subject's plasma arginine level prior to the administration of the subsequent doses is lower than about 50 M, optionally lower than about 40 μM.
A7. The method of any one of embodiments A4-A6, wherein the subsequent doses of the pegzilarginase are adjusted between 0.01 mg/kg and 0.1 mg/kg each time based on the subject's plasma arginine level resulting from the prior dose.
A8. The method of embodiment A7, wherein the subsequent doses of the pegzilarginase are adjusted 0.05 mg/kg each time based on the subject's plasma arginine level resulting from the prior dose.
A9. The method of any one of embodiments A1-A8, wherein the subject does not receive pre-medication before the initial dose of the pegzilarginase.
A10. The method of any one of embodiments A1-A9, wherein the subject's plasma arginine level is at least 250 μM before the initial dose of the pegzilarginase.
A11. The method of any one of embodiments A1-A10, wherein the subject is at least 2 years old.
A12. The method of any one of embodiments A1-A11, wherein the subject is classified as GMFCS Level II, III, IV, or V.
A13. The method of any one of embodiments A1-A12, wherein the subject is co-administered an ammonia scavenger.
A14. The method of any one of embodiments A1-A13, wherein the subject's intake of natural protein is increased after one or more doses of the pegzilarginase.
A15. The method of any one of embodiments A1-A14, wherein the subject's relative intake of essential amino acid mixture compared to natural protein is reduced after one or more doses of the pegzilarginase.
A16. The method of any one of embodiments A1-A15, wherein after one or more doses of the pegzilarginase, the primary source of the subject's protein intake is changed from an arginine-free (or low arginine) essential amino acid mixture to the addition of any food item that contains protein.
A17. The method of any one of embodiments A1-A15, wherein after one or more doses of the pegzilarginase, the primary source of the subject's protein intake is changed from an arginine-free (or low arginine) essential amino acid mixture to natural protein.
A18. The method of any one of embodiments A1-A17, wherein the subject's intake of total protein is greater than 40 g/day after one or more doses of the pegzilarginase.
A19. The method of any one of embodiments A1-A18, wherein the subject's intake of total protein increases by at least 15% after one or more doses of the pegzilarginase.
A20. The method of any one of embodiments A1-A19, wherein the subject's intake of natural protein is increased to at least 50% of the intake of total protein after one or more doses of the pegzilarginase.
A21. The method of embodiment A20, wherein the subject's intake of natural protein is increased to at least 65% of the intake of total protein after one or more doses of the pegzilarginase.
A22. The method of any one of embodiments A1-A21, wherein the subject's mobility is improved after one or more doses of the pegzilarginase.
A23. The method of any one of embodiments A1-A22, wherein arginine metabolite accumulation is reduced after one or more doses of the pegzilarginase.
A24. The method of any one of embodiments A1-A23, wherein accumulated guanidino compounds in the blood and cerebrospinal fluid of the subject is reduced.
A25. The method of embodiment A24, wherein the guanidino compounds are selected from the group consisting of Alpha-N-Acetylarginine, Alpha-keto-d-Guanidinovaleric Acid, Arginic Acid, and Guanidinoacetic Acid.
A26. The method of any one of embodiments A1-A25, wherein the pegzilarginase is administered at the concentration of 5 mg/mL.
A27. The method of any one of embodiments A1-A26, where the pegzilarginase is administered intravenously.
A28. The method of any one of embodiments A1-A26, where the pegzilarginase is administered subcutaneously.
A29. The method of any one of embodiments A1-A28, wherein the pegzilarginase is stored at the concentration of about 5 mg/mL.
A30. The method of any one of embodiments A1-A29, wherein the pegzilarginase is formulated in a buffer comprising 50 mM sodium chloride, 5 mM potassium phosphate, and 1.5% glycerol (w/v) at a pH of 7.4.
A31. The method of any one of embodiments A1-A30, wherein the pegzilarginase is a liquid.
A32. The method of any one of embodiments A1-A30, wherein the pegzilarginase is lyophilized.
A33. The method of any one of embodiments A1-A32, wherein the pegzilarginase is in a vial or an ampoule.
A34. The method of embodiment A33, wherein the vial or ampoule contains about 2 mg or about 5 mg of the pegzilarginase.

In another set of embodiments (embodiment set B), provided are:
B1. A method of treating Arginase 1 (ARG1) deficiency (ARG1-D) in a subject, the method comprising administering a human arginase 1 to the subject,
1) wherein the human arginase 1 is a pegylated human arginase 1 comprising a cobalt metal cofactor, and
2) wherein the human arginase 1 is administered weekly at a dose of from about 0.05 mg/kg to about 0.2 mg/kg.
B2. The method of embodiment B1, wherein the subject's plasma arginine level is between about 40 μM and about 115 M after 12 doses of the human arginase 1.
B3. The method of embodiment B1 or B2, wherein the initial dose of the human arginase 1 is about 0.1 mg/kg.
B4. The method of any one of embodiments B1-B3, wherein subsequent doses of the human arginase 1 are adjusted based on the subject's plasma arginine level prior to the administration of the subsequent doses.
B5. The method of embodiment B4, wherein the subsequent doses of the human arginase 1 are increased if the subject's plasma arginine level prior to the administration of the subsequent doses is higher than about 115 μM, optionally higher than about 150 μM.
B6. The method of embodiment B4, wherein the subsequent doses of the human arginase 1 are decreased if the subject's plasma arginine level prior to the administration of the subsequent doses is lower than about 50 μM, optionally lower than about 40 M.
B7. The method of any one of embodiments B4-B6, wherein the subsequent doses of the human arginase 1 are adjusted between 0.01 mg/kg and 0.1 mg/kg each time based on the subject's plasma arginine level resulting from the prior dose.
B8. The method of embodiment B7, wherein the subsequent doses of the human arginase 1 are adjusted 0.05 mg/kg each time based on the subject's plasma arginine level resulting from the prior dose.
B9. The method of any one of embodiments B1-B8, wherein the subject does not receive pre-medication before the initial dose of the human arginase 1.
B10. The method of any one of embodiments B1-B9, wherein the subject's plasma arginine level is at least 250 μM before the initial dose of the human arginase 1.
B11. The method of any one of embodiments B1-B10, wherein the subject is at least 2 years old.
B12. The method of any one of embodiments B1-B11, wherein the subject is classified as GMFCS Level II, III, IV, or V.
B13. The method of any one of embodiments B1-B12, wherein the subject is co-administered an ammonia scavenger.
B14. The method of any one of embodiments B1-B13, wherein the subject's intake of natural protein is increased after one or more doses of the human arginase 1.
B15. The method of any one of embodiments B1-B14, wherein the subject's relative intake of essential amino acid mixture compared to natural protein is reduced after one or more doses of the human arginase 1.
B16. The method of any one of embodiments B1-B15, wherein after one or more doses of the human arginase 1, the primary source of the subject's protein intake is changed from an arginine-free (or low arginine) essential amino acid mixture to the addition of any food item that contains protein.
B17. The method of any one of embodiments B1-B15, wherein after one or more doses of the human arginase 1, the primary source of the subject's protein intake is changed from an arginine-free (or low arginine) essential amino acid mixture to natural protein.
B18. The method of any one of embodiments B1-B17, wherein the subject's intake of total protein is greater than 40 g/day after one or more doses of the human arginase 1.
B19. The method of any one of embodiments B1-B18, wherein the subject's intake of total protein increases by at least 15% after one or more doses of the human arginase 1.
B20. The method of any one of embodiments B1-B19, wherein the subject's intake of natural protein is increased to at least 50% of the intake of total protein after one or more doses of the human arginase 1.
B21. The method of embodiment B20, wherein the subject's intake of natural protein is increased to at least 65% of the intake of total protein after one or more doses of the human arginase 1.
B22. The method of any one of embodiments B1-B21, wherein the subject's mobility is improved after one or more doses of the human arginase 1.
B23. The method of any one of embodiments B1-B22, wherein arginine metabolite accumulation is reduced after one or more doses of the human arginase 1.
B24. The method of any one of embodiments B1-B23, wherein accumulated guanidino compounds in the blood and cerebrospinal fluid of the subject is reduced.
B25. The method of embodiment B24, wherein the guanidino compounds are selected from the group consisting of Alpha-N-Acetylarginine, Alpha-keto-d-Guanidinovaleric Acid, Arginic Acid, and Guanidinoacetic Acid.
B26. The method of any one of embodiments B1-B25, wherein the human arginase 1 is administered at the concentration of 5 mg/mL.
B27. The method of any one of embodiments B1-B26, where the human arginase 1 is administered intravenously.
B28. The method of any one of embodiments B1-B26, where the human arginase 1 is administered subcutaneously.
B29. The method of any one of embodiments B1-B28, wherein the human arginase 1 is stored at the concentration of about 5 mg/mL.
B30. The method of any one of embodiments B1-B29, wherein the human arginase 1 is formulated in a buffer comprising 50 mM sodium chloride, 5 mM potassium phosphate, and 1.5% glycerol (w/v) at a pH of 7.4.
B31. The method of any one of embodiments B1-B30, wherein the human arginase 1 is a liquid.
B32. The method of any one of embodiments B1-B30, wherein the human arginase 1 is lyophilized.
B33. The method of any one of embodiments B1-B32, wherein the human arginase 1 is in a vial or an ampoule.
B34. The method of embodiment B33, wherein the vial or ampoule contains about 2 mg or about 5 mg of the human arginase 1.

In yet another set of embodiments (embodiment set C), provided are:
C1. A method of diet liberalization for arginase 1 deficiency patients, comprising administering cobalt substituted recombinant arginase 1 subcutaneously once a week and increasing the intake of natural protein.
C2. The method of embodiment C1, wherein the patient reduces the relative intake of essential amino acids compared to natural protein.
C3. The method of embodiment C1, wherein the patient is administered 0.05 to 0.2 mg/kg cobalt substituted recombinant arginase 1.
C4. The method of any of embodiments C1-C3, wherein the liberalization is the change from an arginine-free (or low arginine) essential amino acid mixture as the primary source of protein intake to the addition of any food item that contains protein.
C5. The method of any of embodiments C1-C3, wherein the liberalization is the change from an arginine-free (or low arginine) essential amino acid mixture as the primary source of protein intake to natural protein as the primary intake of protein.
C6. The method of any of embodiments C1-C5, wherein the patient is an adult and the protein intake is greater than 40 g/day.
C7. The method of any of embodiments C1-C6 wherein the patient is co-administered an ammonia scavenger.
C8. The method of any of embodiments C1-C7, wherein the patient intake of total protein is increased more than 15%.
C9. The method of any of embodiments C1-C8 wherein the patient intake of natural protein is over 50% of total protein intake.
C10. A method for diet modification, wherein patients with ARG1 D are administered cobalt substituted recombinant arginase 1 once a week and natural protein intake is increased to at least 65% of total protein intake.
C11. The method of embodiment C10, wherein the patient is administered 0.05 to 0.2 mg/kg cobalt substituted recombinant arginase 1.
C12. A method for treating protein aversion in a patient with a urea cycle disorder, comprising administering cobalt substituted recombinant arginase 1 as described herein.
C13. A method of treating Arginase 1 deficiency, wherein a subset of patients classified as GMFCS Level II receive cobalt substituted recombinant arginase 1 as described herein.
C14. A method of preventing arginine metabolite accumulation, comprising administering cobalt substituted recombinant arginase 1 as described herein.
C15. A method of reducing accumulated guanidino compounds in the blood and cerebrospinal fluid of a patient in need thereof, comprising administering cobalt substituted recombinant arginase 1 as described herein.
C16. The method of embodiment C15, wherein the guanidino compounds are selected from the group consisting of Alpha-N-Acetylarginine, Alpha-keto-d-Guanidinovaleric Acid, Arginic Acid, and Guanidinoacetic Acid.
C17. The method of embodiment C15 or C16, wherein the patient with accumulated guanidino compounds has a disease selected from the group comprising GAMT, arginase 1 deficiency, S-adenosylhomocysteine hydrolase deficiency, CNS injury, and renal damage.

### EXAMPLES

The following is a description of various methods and materials used in the studies. They are put forth so as to provide those of ordinary skill in the art with a complete disclosure and description of how to make and use the present invention, and are not intended to limit the scope of what the inventors regard as their invention, nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### Example 1: Efficacy and Safety of Pegzilarginase in Patients with Arginase 1 Deficiency

### 1.1 Summary

### Pegzilarginase as a Therapy

Pegzilarginase (Co-ARG1-PEG, AEB1102) is a cobalt-substituted, pegylated human recombinant arginase 1 enzyme with enhanced stability and catalytic activity and an extended half-life.

Pegzilarginase is being investigated as an enzyme therapy for ARG1-D. Biological activity of pegzilarginase has been demonstrated based on non-clinical studies and clinical data from Study CAEB1102-101A (Study 101A) and Study CAEB1102-102A (Study 102A) in patients with ARG1-D. The current study (CAEB1102-300A [Study 300A]) seeks to confirm the clinical utility of pegzilarginase for the treatment of patients with ARG1-D.

Given the characteristics of pegzilarginase and the relatively low doses required to reduce arginine levels into the normal range, both intravenous (IV) and subcutaneous (SC) administration were examined in Study 101A, Study 102A, and Study 300A. Brief descriptions of results from Study 101A and Study 102A are presented below.

Study 101A, a Phase 1/2, open-label study designed to evaluate the safety, tolerability, pharmacokinetics, pharmacodynamics (PD), and proof of concept in patients with ARG1-D, found that IV pegzilarginase was well tolerated and had an acceptable and manageable safety profile. No subject discontinued treatment due to adverse events (AEs) or had detectable anti-drug antibodies (ADAs) at the end of the study. Notably, after 8 weekly doses of treatment in Part 2 of the study, pegzilarginase rapidly and sustainably reduced plasma arginine levels to below the current medical guideline level of 200 μM, and in some subjects, within the normal range (40 to 115 μM). The improved plasma arginine control following pegzilarginase treatment was accompanied by evidence of early clinical benefit in the study, where 9 of 16 subjects showed clinically meaningful improvements in at least 1 clinical outcome. These results suggest that pegzilarginase has the ability to both improve important disease-related abnormalities and to potentially modify disease progression. In addition, in clinical and in in vitro and in vivo settings, pegzilarginase demonstrated approximate dose proportionality with IV and SC administration.

Study 102A, a Phase 2, open-label study designed to evaluate the long-term safety, tolerability, and efficacy of IV and SC pegzilarginase in pediatric and adult subjects with ARG1-D, was an LTE study of Study 101A. All 14 subjects who completed Study 101A continued on to enroll and be treated with pegzilarginase in Study 102A. Interim results of ongoing Study 102A with subjects receiving between approximately 110 and 183 weeks of pegzilarginase administered IV or SC weekly at doses of 0.05 to 0.2 mg/kg demonstrated a consistent and sustained reduction in plasma arginine level to within normal range and associated decreases in plasma guanidino compound level. Clinically meaningful improvements in mobility assessed by neuromotor function were maintained, and importantly, the duration of the long term follow up in this study, was over a timescale where clinical disease progression would typically be observed on current standard of care.

### Rationale for the Phase 3 Study 300A

As described above, pegzilarginase has been shown to produce marked, rapid, and sustained reductions in plasma arginine levels in patients with ARG1-D, allowing substantially improved arginine control relative to what can be achieved with individualized disease management (IDM) approaches. The improved control of plasma arginine levels was accompanied by clinical improvements in one or more instruments of neuromotor function, consistent with the hypothesis that improved plasma arginine control has the potential to slow disease progression in affected patients. The Phase 3 study (Study 300A) was designed to assess the safety and efficacy in lowering arginine levels and improving clinical outcomes of pegzilarginase compared to placebo. The goal of the current study, Study 300A, was to confirm these preliminary findings in a blinded, placebo-controlled design and to gain further understanding of the impact of pegzilarginase on a broader range of disease manifestations. Interim analysis results as of the data cutoff date are presented in this report.

### Study Objectives

The Phase 3 PEACE study was designed to assess the safety and efficacy of the ability of pegzilarginase to achieve arginine control and to evaluate the relationship between arginine lowering and improvements in neuromotor function/mobility.

### Study Design

The study consists of a randomized, placebo-controlled, double-blind treatment period of 24 weeks followed by an open-label LTE period of up to 150 weeks in which all subjects receive active pegzilarginase. Pegzilarginase or placebo was given in a 2:1 ratio of weekly IV infusions plus IDMs for the first 24 weeks of the Double-blind period. After completion of the 24-week DB, subjects transitioned to the LTE period, Investigators remained blinded to the randomized treatment for the first 8 weeks, however, all subjects received pegzilarginase during LTE period. Subjects were transitioned from IV administration to weekly SC dosing anytime 8 weeks in the LTE to enable ease and convenience of administration during the remainder of the LTE.

### Relevance of the Key Mobility Outcomes

Although not studied formally in patients with ARG1-D prior to initiation of the clinical development program for pegzilarginase, the capacity for mobility is important for independence and declines clinically over time with disease progression in ARG1-D. Evidence-based outcome measures are necessary to monitor changes in patient performance, can be used to support the need for continued home health care, or indicate the need for additional support services or devices needed for ambulation. Given the paucity of data on the utility of neuromotor assessments in the assessment of disease burden and treatment effects in ARG1-D, a number of assessments were included to further study the impact of pegzilarginase on neuromotor outcomes including mobility in this ultra-rare disease. Patients with ARG1-D may have varying degrees of impairment, and complimentary assessments such as the 2MWT and GMFM-E were considered to be helpful as key secondary endpoints in assessing a broader population in the context of a clinical study.

The 2MWT and GMFM-E were utilized in the prior clinical studies in ARG1-D. Based on insights from the Phase 1/2 and Open Label Extension study for pegzilarginase two key mobility outcomes, the 2MWT and GMFM-E, were identified and selected to assess the clinical benefits of pegzilarginase beyond arginine reduction in the Phase 3 study. The 2MWT is a relevant tool to measure the performance outcome of mobility, being a shorter modification of the 6-minute version (e.g., NIH Toolbox motor domain-2MWT [Bohannon 2014]). The 2MWT has been validated over a large spectrum of age groups, including children (Bohannon 2018) and adults (Bohannon 2014). Furthermore, both the 6-minute walk test and a shorter modification, the 2MWT, have been used successfully in children (Geiger 2007, Maher 2008) and in adults with cerebral palsy (Andersson 2006, Ammann-Reiffer 2018), which is also characterized by spasticity. The 2MWT is limited as a tool to subjects with the ability to walk, and the cognitive ability and focus to understand and follow directions. The second outcome, the GMFM-E, has been used in similar populations, such as cerebral palsy, to assess functional mobility that is impacted by ARG1-D. This measure assesses the ability to walk, run and climb stairs and may be particularly relevant for those more severely affected. This tool is limited to measure improvement for those with less disability as they are often at or near the top score. This suggests that while the sensitivity of the test is good, there is greater opportunity to measure increases in score is subjects with more advanced disease who are capable of safely being assessed.

### Study Conduct

The study was conducted during the COVID-19 pandemic, which required changes to the conduct of the study as a result of site closures and stay home measures to prevent the spread of illness. This required the implementation of pauses on an individual subject level based on the period of the study each subject was in when site closures occurred. These were done systematically and were documented in study documents. The number of missed visits and assessments due to COVID-19 was small in comparison to the total number planned. Every attempt was made to ensure data collection was robust, assessments were done with accuracy and collected correctly and on time when permitted. Despite the pandemic, the study was well executed, and subject safety and data integrity was maintained. Overall, the COVID-19 pandemic did not significantly impact the ability to monitor and manage subject safety, data integrity, or assessment of efficacy as of the data cutoff.

### Efficacy Results

Once weekly pegzilarginase administration resulted in early, consistent, and sustained reductions in plasma arginine levels through Week 24 of the Double-blind period compared to no meaningful change in the placebo group. Previous guidelines recommended targeting control of plasma arginine levels to <200 μM, as levels below this threshold were previously unattainable for most patients on a consistent basis in clinical practice with dietary restriction alone. Treatment with pegzilarginase resulted in normalization of arginine levels in the vast majority of subjects (90.5%) through 24 weeks of dosing. Subjects in the Double-blind period continued on study in the LTE period and transitioned from IV dosing to SC dosing at the same IV dose, unless dictated by arginine levels, for ease of administration and enhanced convenience. Continued administration of pegzilarginase via the SC route of administration in those who received pegzilarginase in the Double-blind period resulted in similarly consistent and sustained reduction in plasma arginine level through Week 24 in the LTE period. Pegzilarginase-naïve subjects initially randomized to placebo initiated weekly pegzilarginase in the LTE, moving to SC administration after 8 weeks, and 100% of those reaching LTE24 (N=11) were also able to normalize plasma arginine levels.

A key goal, rarely obtainable with current treatment approaches, is to achieve arginine levels to meet the guideline recommended level (<200 μM) and ideally to normal levels. At Week 24 of the Double-blind period, over 90% of subjects treated with pegzilarginase achieved arginine values within the treatment guidelines (<200 μM) compared to none in the placebo group. In addition, over 90% of subjects treated with pegzilarginase achieved arginine values within the normal range (40 to 115 μM) at Week 24 of the Double-blind period, which is unprecedented in this subject population.

Key secondary efficacy endpoints focused on neurologic/neuromotor outcomes. Given the wide age range of subjects recruited into this study and the progressive nature of the disease, the degree of Baseline deficits in neuromotor function varied across subjects. Positive effects on neuromotor function, as assessed by GMFM-E and the 2MWT, were observed in the pegzilarginase group. Similar proportions of subjects in the pegzilarginase group demonstrated clinically relevant and/or numerical increases in GMFM-E and 2MWT assessments (approximately 55% [N=11] and 50% [N=10], respectively). Improvements in neuromotor function were also observed in subjects in the placebo group but were generally at a lower magnitude than those observed in the pegzilarginase group. Pegzilarginase administration resulted in longer increases from Baseline in the mean distance walked over 2 minutes (2MWT) and greater improvements from Baseline in GMFM-E scores at Week 24 compared to placebo. Numerical improvements were observed in in both the GMFM-E and the 2MWT, and for GMFM-E there was a positive trend (p=0.1087) in change from Baseline compared to placebo. In the subgroup analysis, subjects treated with pegzilarginase with more severely restricted mobility (GMFCS≥II) had greater gains in both the 2MWT and the GMFM-E than subjects classified as GMFCS I, as they were not limited by the maximal improvement threshold or ceiling of the assessment. These differences may be a reflection of a greater effectiveness of capturing clinical benefit with these outcome tools in patients with more advanced disease. The improvements in patients with more advanced disease point to the clinical utility for treatment with pegzilarginase in these patients. Given the limitations of these outcome tools, the less marked improvement in those with less advanced disease does not preclude the potential for prevention of progression which is also a treatment goal. Some of the limitations of these specific clinical assessments in some segments of the population include the following. For the 2MWT, subjects with a classification of GMFCS IV were generally wheelchair bound and were severely limited in their ability to walk if they could ambulate at all (N=4). For the GMFM-E assessments, in those with a classification of GMFCS I, with less severe disability, measurement of improvement was limited by the upper boundary of the test as some were close to or at the ceiling or upper boundary of response at Baseline. In further demonstration of the treatment effect of pegzilarginase during the Double-blind period, for subjects completing LTE24, increases from Baseline for 2MWT and GMFM-E continued to be observed in subjects treated with pegzilarginase, highlighting continued improvement with further therapy, while subjects who transitioned from placebo to pegzilarginase were stable or made improvements.

Based on interim results of ongoing Study 300A after 24 weeks of treatment in the Double-blind period, pegzilarginase, administered IV weekly at doses ranging from 0.05 to 0.20 mg/kg, met the primary endpoint (p<0.0001). Pegzilarginase demonstrated a consistent and sustained reduction in plasma arginine to both therapeutic and normal levels while no meaningful changes were observed in the placebo group.

The reductions in plasma arginine were associated with clinically relevant improvements in mobility. Pegzilarginase demonstrated a positive trend in GMFM-E compared to placebo and numerical improvements in the 2MWT compared to placebo.

More subjects treated with pegzilarginase met response criteria across multiple domains, with generally greater magnitude of response compared to placebo treated subjects.

Pegzilarginase was well tolerated in subjects with ARG1-D. The TEAEs were transient, manageable, and were self-limiting or resolved with standard medical care.

In the LTE period, subjects switched to SC dosing, to simplify dosing and improve convenience of therapy, at the same IV dose unless dictated by arginine levels, with exposures for up to 104 weeks. The safety profile was consistent between IV and SC dosing

During the LTE period, subjects continued to maintain normal arginine levels after transitioned to SC dosing and maintained or further improved mobility while those transitioning from placebo to pegzilarginase were stable or demonstrated improvements.

The safety profile was consistent between the DB and LTE periods.

In summary, pegzilarginase represents a novel targeted treatment option to address the unmet medical need in ARG1-D, with the totality of safety and efficacy data demonstrating a positive benefit-risk profile for patients affected by this rare progressive and debilitating disease.

### 1.2 Background and Overview of Clinical Efficacy

Pegzilarginase (Co-Arg1-PEG, AEB1102) is a cobalt-substituted, pegylated human recombinant arginase 1 enzyme with enhanced stability, more potent catalytic activity, and an extended half-life compared to the native enzyme. Pegzilarginase cleaves excess plasma arginine, to ornithine and urea, the natural breakdown products of arginase 1.

ARG1-D is a rare, debilitating, progressive, inherited, neurotoxic, metabolic disease associated with increased arginine and its metabolites, with significant reductions in quality of life, increased morbidity, and premature mortality (Diez-Fernandez 2018; Schlune 2015; Summar 2013; Waisbren 2018).

There are similarities with another inherited metabolic disease, phenylketonuria (PKU), and arginase 1 deficiency (ARG1-D) in terms of inherited liver enzyme deficiency and amino acid accumulation in the blood. Phenylketonuria is a deficiency in phenylalanine hydroxylase (PAH) in the liver that results in high levels of circulating amino acid phenylalanine (Phe) that, although not well understood, leads to chronic neurologic and psychiatric disease when left untreated or undertreated. Treatment is focused on lowering Phe plasma levels to improve clinical outcomes. Pegvaliase is an approved novel enzyme substitution therapy that helps address a significant unmet need in PKU patients who have been unable to control their blood Phe levels with current treatment options.

Akin to PKU, in ARG1-D, the enzyme deficiency in the liver results in high plasma and tissue levels of arginine resulting in spasticity, gait disorders, difficulty walking, and developmental and cognitive disability when treated with the current standard of care. Similarly, pegzilarginase, a human arginine-metabolizing enzyme intended to be used as enzyme substitution therapy in patients with ARG1-D, cleaves excess plasma arginine, to ornithine and urea, the natural breakdown products of arginase 1. The goal of therapy is to lower blood arginine concentrations to <200 μM to minimize the toxic impacts of excess arginine, excess guanidino compounds (GCs) derived from arginine, and the dysregulation of nitric oxide metabolism (Palynziq USPI).

Manifestations of ARG1-D often appear early in childhood and include neuromotor and neurocognitive deficits. The clinical picture is ultimately strikingly uniform, characterized by spasticity, gait disorders, difficulty walking, and developmental and cognitive disability, with some patients having seizures, although variation in timeframe and progression of symptoms has been observed between patients (De Deyn 1997; Marescau 1990; Waisbren 2018).

Elevated arginine and its metabolites are believed to be the key contributors to disease manifestations and progression (De Deyn 1997; Marescau 1990; Waisbren 2018). Pre-clinical models, through gene expression profiling of the motor cortex of an ARG1-D mouse knockout model, have shown that arginase deficiency fosters demyelination of the corticospinal tract during postnatal central nervous system development, with altered synapse density in the motor cortex. Several other diseases correlate spasticity with myelination loss, via multiple etiologies, including multiple sclerosis and hereditary spastic paraplegia. Taken together, the literature findings support both the importance of arginine with subsequent metabolite accumulation as the key driver of the neurological manifestations of ARG1-D and illustrate the effectiveness of reducing arginine to reverse the neurologic damage, through enzyme administration or hepatic expression of ARG1, for functional recovery of oligodendrocytes to improve central nervous system abnormalities.

Neuromotor complications of the disease are a hallmark feature, and the lower limb spasticity typically seen in early childhood impairs mobility and balance, leading to gait disorders with difficulties in walking and climbing stairs. Patients typically present with some form of lower limb spasticity with approximately 60% to 75% having spasticity at initial presentation, which increases with extended follow-up (Carvalho 2012; Huemer 2016). This progressive spastic diplegia results in increasing immobility, inability to perform activities of daily living, and increased caregiver burden.

There are currently no approved therapies for the treatment of ARG1-D that significantly reduce plasma arginine, and dietary intervention rarely achieves treatment guideline-directed goals, resulting in continued disease progression. Pegzilarginase represents a novel targeted treatment option to address the unmet medical need for patients affected by ARG1-D. The totality of the efficacy data for pegzilarginase, ranging from 24 to over 120 weeks in duration, demonstrates substantial evidence of effectiveness by achieving sustained plasma arginine levels that meet or exceed therapeutic guidance (<200 μM) and are within normal levels (40 to 115 μM) for most patients, with clinically meaningful improvements in mobility at the study level and more importantly at the individual subject level in this ultra-rare disease.

### Diagnosis

Diagnosis can be readily made with routinely available plasma arginine assessment or genetic analysis (Häberle 2012; Haberle 2019). Delays in diagnosis may occur due to overlap in symptomatology with other developmental diseases, such as cerebral palsy or hereditary spastic paraplegia (Carvalho 2012).

### Current Standard of Care

There are no pharmacologic agents known to effectively reduce arginine levels in patients with ARG1-D, and no agents have been approved in any country that specifically target the enzyme deficiency. Current therapies include low protein diet that modestly lowers arginine levels, and in some cases, this was accompanied by improved neuromotor functioning (Häberle 2012; Huemer 2016; Lambert 1991; Prasad 1997). Pegzilarginase represents the first potential enzyme therapy for ARG1-D. Literature indicates that modest lowering of plasma arginine levels achieved with severe dietary protein restriction can lead to improvements in disease manifestations; however, the currently recommended treatment goal of reducing plasma arginine to <200 μM is difficult to achieve, in part, because adhering to a rigorous diet to reduce arginine levels to within or close to the normal range is challenging (Häberle 2012; Huemer 2016; Lambert 1991; Prasad 1997; Burrage 2015). Substantial lowering of plasma arginine levels with pegzilarginase, therefore, has the potential to slow or halt the progression of neuromotor, neurocognitive, and/or adaptive behavioral deterioration in patients with ARG1-D.

The current treatment of ARG1-D is focused on lowering plasma arginine levels through lifelong dietary protein restriction. Protein intake is limited to the minimum required to maintain protein biosynthesis and growth. Typically half or more of dietary protein is given in the form of an arginine-free essential amino acid (EAA) mixture. Such dietary modification can produce modest reductions in plasma arginine levels, but levels remain markedly elevated in most patients, likely due to the fact that arginine flux is largely dependent on whole body protein turnover and minimally affected by dietary intake (Wu 1998). Moreover, the diet is unpalatable and difficult to maintain and manage, especially in growing children. Ammonia scavengers may be used to help control ammonia levels, and therefore the number and severity of episodes of hyperammonemia that occur in some patients with ARG1-D. Liver transplantation has been reported to achieve disease normalization in some patients, and despite limited successes with this intervention, transplantation is available to only a small fraction of patients and carries a substantial risk of mortality and morbidity.

### Unmet Medical Need

The lack of treatment options for patients deficient in Arginase 1 that can effectively lower arginine levels or frequently achieve reductions to within the normal range and promote the lifelong maintenance of normal arginine levels highlights the unmet need for a new therapy. The development of such a therapeutic could be useful in the attempt to minimize exposure to the neurotoxic effects of arginine and its metabolites and offer the potential for normal neurocognitive and neuromotor development in these patients (Amayreh 2014).

### 1.3 Selection of Study Population

### Inclusion Criteria

For inclusion in the study, subjects were required to fulfill all of the following criteria:
1. The subject and/or parent/guardian provided written informed consent/assent, which included compliance with the requirements and restrictions listed in the ICF and in the protocol.
2. A current diagnosis of ARG1-D as documented in medical records, which included 1 of the following: elevated plasma arginine levels, a mutation analysis that resulted in a pathogenic variant, or RBC arginase activity. For entry into this study, subjects also had to fulfill the following plasma arginine criteria:
   a. The average of all measured values of plasma arginine during the Screening Period prior to the randomization visit (Visit 1, Study Day 1) was ≥250 μM.
   b. If a subject was re-screened, the only values that were considered for eligibility assessment were those in the current Screening Period.
3. Subjects were ≥2 years of age on the date of informed consent/assent.
4. The subject was assessable for clinically meaningful within-subject change (clinical response) on at least 1 component of 1 assessment included in the key secondary/other secondary endpoints.
5. Have received documented confirmation from the Investigator and/or dietician that the subject could maintain their diet in accordance with dietary information presented in the protocol, i.e., could maintain the current level of protein consumption, including natural protein and EAA supplementation.
6. Subjects receiving ammonia scavenger therapy, anti-epileptic drugs, and/or medications for spasticity (e.g., baclofen) were on a stable dose of the medication for at least 4 weeks prior to randomization and were willing to remain on a stable dose during the double-blind portion and blinded follow-up portions of the study.
7. Were female and male subjects. Female subjects of childbearing potential had a negative serum pregnancy test during the Screening Period before receiving the first dose of study treatment, and a negative urine pregnancy test on the day of the first dose, prior to the first dose. If the subject (male or female) was engaging in sexual activity that could lead to pregnancy, he or she were surgically sterile, postmenopausal (no menses for 12 months without an alternative medical cause or a high follicle-stimulating hormone level in the postmenopausal range in women not using hormonal contraception or hormonal therapy), or agreed to use a highly effective method of birth control during the study and for a minimum of 30 days after the last study medication administration. Highly effective methods of contraception included: combined (estrogen and progestogen containing) hormonal contraception associated with inhibition of ovulation; progesterone-only hormonal contraception associated with inhibition of ovulation; intrauterine device; intrauterine hormone-releasing system; or abstinence (refraining from heterosexual intercourse during the entire period of risk associated with study treatment).

### Exclusion Criteria

Subjects meeting any of the following criteria were excluded from the study:
1. Had a hyperammonemic episode (defined as an event in which a subject had an ammonia level ≥100 M with 1 or more symptoms related to hyperammonemia requiring hospitalization or emergency room management) within the 6 weeks before the first dose of study medication was administered.
2. Had an active infection requiring anti-infective therapy within 3 weeks prior to first dose.
3. Had known active infection with human immunodeficiency virus, hepatitis B, or hepatitis C.
4. Had extreme mobility deficit, defined as either the inability to be assessed on the Gillette Functional Assessment Questionnaire (GFAQ) or a score of 1 on the GFAQ.
5. Had other medical conditions or comorbidities that, in the opinion of the Investigator would interfere with study compliance or data interpretation (e.g., severe intellectual disability precluding required study assessments).
6. Had participated in a previous interventional study with pegzilarginase.
7. Had a history of hypersensitivity to polyethylene glycol (PEG) that, in the judgment of the Investigator, put the subject at unacceptable risk for AEs.
8. Subject was being treated with botulinum toxin-containing regimens or plans to initiate such regimens during the DB or blinded follow-up portions of the study or received surgical or botulinum toxin treatment for spasticity-related complications within the 16 weeks prior to the first dose of study treatment in this study.
9. Was currently participating in another therapeutic clinical study or had received any investigational agent within 30 days (or 5 half-lives whichever is longer) prior to the first dose of study treatment in this study.
10. Previous liver or hematopoietic transplant procedure.

### 1.4 Study Objectives and Endpoints

The overall purpose of this study was to confirm the safety, tolerability, and efficacy of long-term IV and SC administration of pegzilarginase for the treatment of pediatric and adult subjects with ARG1-D and hyperargininemia. The study objectives and endpoints are presented below in Table 1. This study was designed to assess the effect of pegzilarginase in combination with subjects' IDM plans.

TABLE 1

| | |
|---|---|
| Objectives | Endpoints |

PRIMARY

| | |
|---|---|
| To demonstrate the efficacy of | Change from Baseline in plasma arginine |
| pegzilarginase relative to placebo based on | after 24 weeks of study treatment |
| a statistically significant decrease in plasma | |
| arginine concentrations | |

KEY SECONDARY

| | |
|---|---|
| To demonstrate the efficacy of | Mean change from Baseline at Week 24 in |
| pegzilarginase relative to placebo based on | the 2MWT |
| key mobility outcome measures | Mean change from Baseline at Week 24 in |
| | the GMFM-88 Part E |

SECONDARY
To further demonstrate the clinical and biochemical efficacy of
pegzilarginase relative to placebo

| | |
|---|---|
| To compare pegzilarginase with placebo | A subject is considered a responder if their |
| with respect to the proportion of subjects | endpoint arginine value is <200 μM after 24 |
| whose endpoint arginine value falls below | weeks of study treatment, and a non- |
| target guidance of 200 μM | responder otherwise |
| To compare pegzilarginase with placebo | A subject is considered a responder if their |
| with respect to the proportions of subjects | endpoint arginine value is in the normal |
| whose endpoint arginine value falls within | range (≥40 μM to ≤115 μM) after 24 weeks |
| the normal range of 40 to 115 μM | of study treatment, and a non-responder |
| | otherwise |
| To compare pegzilarginase with placebo for | Change from Baseline in ornithine and |
| changes in ornithine and guanidino | guanidino compounds after 24 weeks of |
| compounds | study treatment |
| To compare pegzilarginase with placebo | Mean change from Baseline at Week 24 in |
| with respect to other aspects of mobility | the GMFM-88 Part D |
| | Mean change from Baseline at Week 24 in |
| | the FMA (FMS and GFAQ) |
| To compare pegzilarginase with placebo | Mean change from Baseline at Week 24 in |
| with respect to adaptive behavior | the VABS-II |
| To evaluate the safety and immunogenicity | AEs and ADAs were collected |
| of pegzilarginase | |
| To further characterize the PK profile of | PK and PD data were analyzed |
| pegzilarginase | |

### 1.5 Overall Study Design and Plan

This study (Study 300A) was a multi-center, randomized, double-blind (DB), placebo-controlled study designed to evaluate the safety and efficacy of pegzilarginase and planned to be conducted in approximately 30 subjects with ARG1-D, with the total duration of the study expected to be approximately 178 weeks per subject. This study consisted of the following:
1. A screening period of 3 to 4 weeks in duration to collect all necessary information to ensure the subjects met study eligibility criteria and to establish Baseline plasma arginine data, collect prescribed diet data, and determine adherence to prescribed diet using a diet diary.
2. A randomized, DB treatment period of 24 weeks.
3. An open-label long-term extension (LTE) period of up to approximately 150 weeks in which all subjects received active pegzilarginase. The first 8 weeks of treatment were to remain blinded to ensure that study data relating to the randomized period is collected prior to unblinding.

Subjects were randomized to treatment following completion of all screening assessments and confirmation of study eligibility in a 2:1 ratio to receive weekly IV infusions of pegzilarginase plus IDM or placebo plus individualized disease management (IDM) during the 24-week double blind treatment period.

Upon completion of the 24-week double-blind treatment period of the study, all eligible subjects began an 8-week blinded follow-up treatment period during which all subjects received pegzilarginase. Subjects initially randomized to pegzilarginase received the optimized dose they received during the 24-week double-blind treatment period (which was permitted to be adjusted as clinically indicated). Subjects initially randomized to placebo during the 24-week double blind treatment period began the 8-week DB treatment period at a dose of 0.10 mg/kg that was permitted to be adjusted during the LTE period based on arginine levels.

After the first 8 weeks of the blinded LTE period, subjects had the option to receive pegzilarginase by weekly subcutaneous (SC) administration, with Investigator and sponsor approval. The first 4 SC doses were given at the investigational site. The initial mg/kg SC dose was permitted to be the same as the IV dose and could be adjusted based on arginine levels. Subsequent SC doses may have been administered outside of the investigational site by appropriately trained home healthcare personnel if considered safe and appropriate in the opinion of the Investigator in consultation with the sponsor. During the LTE period, all subjects received weekly pegzilarginase plus IDM.

### Study Design Schema

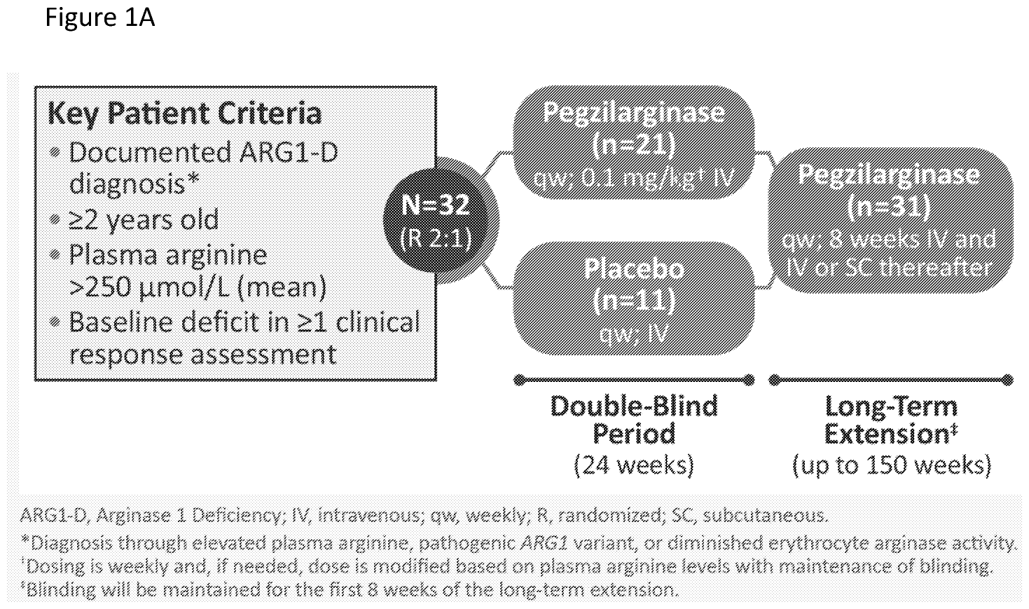

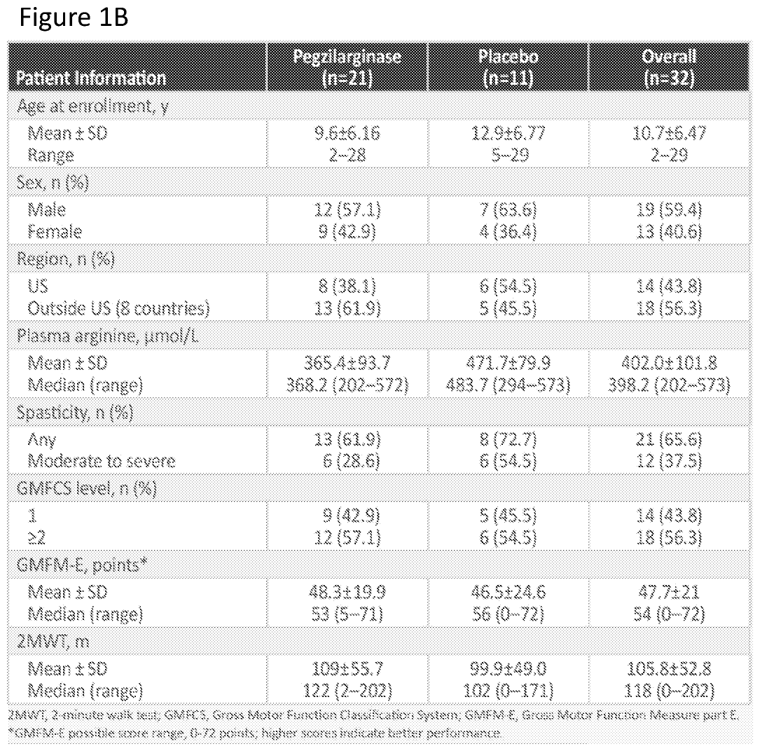

The study design schema is presented in FIG. 1A. The key demographics and baseline clinical characteristics are presented in FIG. 1B.

A total of 32 patients were enrolled (pegzilarginase, n=21; placebo, n=11). Patients were racially and ethnically diverse; approximately 30% were Hispanic or Latino. The average of all measured values of plasma arginine during the Screening Period prior to the randomization visit was ≥250 μM. All patients were managed with dietary protein restriction. Spasticity was evident in 65.6% of patients (n=21/32) and functional impairment of GMFCS level >2 was evident in >50% of patients. Subjects receiving ammonia scavenger therapy, anti-epileptic drugs, and/or medications for spasticity (e.g., baclofen) were on a stable dose of the medication for at least 4 weeks prior to randomization and were willing to remain on a stable dose during the double blind portion and blinded follow-up portions of the study.

### Individualized Disease Management

This study was designed to assess the effect of pegzilarginase in combination with subjects' IDM plans, which typically included a prescribed diet with severe protein restriction and EAA supplementation and/or the use of ammonia scavengers.

Subjects had stable IDM plans, as demonstrated during the Screening Period prior to study participation, including the amount of prescribed protein and the amount of prescribed EAAs and/or the use of a prescribed dose of ammonia scavenger medication, if applicable. Subjects were willing and able to maintain consistent dietary protein intake during the DB portion of the study. Subjects and/or their caregivers were counseled by the Investigator and dietician not to modify their IDM during the DB portion of the study unless there was a clear medical reason to do so. Compliance with IDM was captured in the electronic case report form (eCRF).

Subjects were required to have a stable, consistent diet for the entire duration of the blinded period, which included the 24-week randomization period and the first 8 weeks of the open-label period of the study. A consistent diet was defined as one in which the prescribed natural protein medical food EAA supplementation and/or the use of ammonia scavengers, and caloric intake did not change more than 15% from Baseline. Any prescribed changes of more than 15% from Baseline were documented.

### Treatments Administered

Subjects were randomized to treatment following completion of all screening assessments and confirmation of study eligibility in a 2:1 ratio to receive weekly IV infusions of pegzilarginase plus IDM or placebo plus IDM during the 24-week DB treatment period.

Upon completion of the 24-week, DB treatment period of the study, all eligible subjects began the 8-week, blinded follow-up treatment period, during which all subjects received pegzilarginase.

Subjects initially randomized to pegzilarginase received the optimized dose they received during the 24-week, DB treatment period (which was adjusted as clinically indicated). Subjects initially randomized to placebo during the 24 week, DB treatment period began the 8 week blinded treatment period transitioned to pegzilarginase at a dose of 0.10 mg/kg, which was adjusted during the LTE period based on arginine levels. All subjects had their final DB assessments (i.e., Week 24 assessments) prior to initiation of pegzilarginase dosing in the LTE period.

After the first 8 weeks of the blinded LTE period, subjects had the option to receive pegzilarginase by SC administration, with Investigator and sponsor approval. The first 4 SC doses were to be given at the investigational site. The initial mg/kg SC dose was the same as the IV dose. Subsequent SC doses were permitted to be administered outside of the investigational site by appropriately trained home health care personnel if considered safe and appropriate in the opinion of the Investigator in consultation with the sponsor. During the LTE period, all subjects received pegzilarginase plus IDM.

The unit dose strength of the formulation used for IV administration was 1 mg/ml. The unit dose strength of the formulation used for the SC administration was 5 mg/ml. Pegzilarginase is a cobalt-substituted, pegylated, recombinant human arginase 1. Pegzilarginase is formulated in 50 mM NaCl, 1 mM K₂HPO₄, 4 mM KH₂PO₄, and 1.5% w/v glycerol. Premedication with a non-sedating antihistamine was recommended 30 minutes prior to the start of dosing. Starting dose of 0.10 mg/kg; dose adjustment allowed based on pharmacodynamic response. The IV infusion time is approximately 30 minutes.

### Dose Selection

For subjects randomized to pegzilarginase, the weekly IV starting dose of 0.10 mg/kg was selected based on available safety, PK, PD, and clinical results (from Study 101A), which indicated that this dose was well tolerated and was anticipated to provide a desirable level of plasma arginine control in the majority of the target population.

The final dose in Study 101A was between 0.04 and 0.20 mg/kg for the 14 subjects who had completed the study at the time of database lock. The median final dose was 0.09 mg/kg; thus, the starting dose of 0.10 mg/kg in this study was selected as being representative of an effective and well tolerated dose. Dose modifications were made based on plasma arginine levels. In general, the dose was modified if a subject's plasma arginine level at 168 hours post dosing was outside the range of 50 to 150 μM.

### Efficacy Assessments

The efficacy of pegzilarginase was measured by the magnitude, onset, and duration of changes in plasma arginine levels, as well as the following clinical outcome assessments:
Neurological and neuromotor manifestations
Adaptive behavior changes
Caregiver and Clinician Global Impressions of Change and Severity and Exit Interview.
Other clinical outcomes, such as frequency of seizures and hyperammonemic episodes
Neurocognitive, developmental, and quality of life measures.
Ornithine and guanidino compounds and other exploratory biomarkers (i.e., guanidinoacetic acid, α-N-acetylarginine, α-keto-δ-guanidinovaleric acid, argininic acid, creatine, or others)

In addition to the neurological examination performed with the physical examination, several assessments of neurological and neuromotor functions were tested: the 2MWT, Functional Mobility Assessment (FMA; Functional Mobility Scale [FMS] and GFAQ), 9-Hole Pegboard Test, GMFM-D and GMFM-E, and Modified Ashworth Scale (MAS).

Blood samples for analysis of plasma arginine, ornithine, and guanidino compounds were collected. Guanidino compounds to measured included guanidinoacetic acid, α-N-acetylarginine, α-keto-δ-guanidinovaleric acid, argininic acid, and creatine.

### Electroencephalograms

In addition to clinically overt seizures, a high frequency of abnormal electroencephalogram (EEG) activity has been observed in patients with ARG1 D (Carvalho 2012; Huemer 2016; De Deyn 1997). Electroencephalography is a test used to evaluate electrical activity in the brain. The EEG is defined as electrical activity of an alternating type recorded from the scalp surface after being picked up by metal electrodes and conductive media (Teplan 2002). Electroencephalogram results have been shown to be abnormal in a considerable proportion of patients with ARG1 D, with EEG recordings showing slow diffuse activity and generalized epileptic activity (Carvalho 2012; De Deyn 1997).

In this study, all subjects were consented for Baseline and follow-up routine (i.e., not sleep, ambulatory, or video telemetry) outpatient EEG recordings.

Electroencephalograms (EEGs) were performed at Screening and Week 24 for all subjects. An EEG result was interpreted as normal, abnormal not clinically significant (NCS), or abnormal clinically significant (CS). If a result was reported as abnormal CS, then the abnormality was reported.

A subject whose EEG presents abnormalities with a reported result of abnormal CS was listed for the DB analysis period. No other summary reports or listings were generated for these data.

### Clinical Laboratory Assessments

A full range of clinical markers as shown in Table 2 below were assessed for all trial subjects.

TABLE 2

| Panel | Tests |
|---|---|
| Hematology | RBCs, WBCs, hemoglobin, hematocrit, WBC differential count |
| | (neutrophils, lymphocytes, eosinophils, and basophils), platelet count, |
| | mean corpuscular hemoglobin concentration, mean corpuscular |
| | hemoglobin, mean corpuscular volume, mean platelet volume, and |
| | RBC distribution width. |
| Serum chemistry | Sodium, potassium, chloride, bicarbonate, calcium, phosphate, BUN, |
| | creatinine, glucose, lactate dehydrogenase, AST, ALT, ALP, total |
| | bilirubin, total protein, and albumin. |
| Other biomarkers | Growth hormone, IGF-1. |
| | Baseline only: S-transthyretin/prealbumin; S-transferrin/TIBC, sRBP. |
| Arginine, | Arginine, ornithine, guanidinoacetic acid, α-N-acetylarginine, |
| ornithine, | α-keto-δ-guanidinovaleric acid, argininic acid, creatine, |
| guanidino | homoarginine, or others. |
| compounds | |
| Amino acids | Taurine, aspartic acid, hydroxyproline, threonine, serine, asparagine, |
| | glutamic acid, glutamine, proline, glycine, alanine, citrulline, valine, |
| | methionine, cystine, isoleucine, leucine, tyrosine, phenylalanine, a- |
| | amino-n-butyric acid, tryptophan, lysine, and histidine. |
| Ammonia (local | Plasma ammonia was measured at Baseline and at regular intervals |
| laboratory) | throughout the study. Samples were analyzed shortly after collection |
| | by the local laboratory due to the lability of this sample. |
| Urinalysis | Appearance, glucose, ketones, blood, protein, nitrate, bilirubin, |
| | specific gravity, pH, and leukocyte esterase. If positive for blood, |
| | protein trace, or leukocyte esterase then microscopy was included. |
| Coagulation | aPTT, PT/INR. |
| Pregnancy testing | Serum pregnancy tests were performed by the central laboratory for |
| | all female subjects of childbearing potential at the Screening and End |
| | of Study visits. Serum or urine pregnancy tests were performed at the |
| | local laboratory as noted at other visits as described in the schedules |
| | of assessments. Subjects who had a confirmed positive pregnancy test |
| | at any time during the study were discontinued from further study |
| | treatment administration and were followed for pregnancy outcome. |
| Mutation analysis | Mutation analysis of the arginase 1 gene and arginase activity in |
| and arginase | RBCs were assessed for all subjects at the central laboratory. |
| activity in RBCs | Mutation analysis was required for all subjects in accordance with |
| | local or country laws or regulations. If the subject's medical record |
| | contained the results of a previous mutation analysis, that was |
| | sufficient. Some countries may have required a separate, optional |
| | consent/assent; if a subject did not consent/assent, they could still be |
| | enrolled in the study. |
| Phenylbutyrate | Samples for phenylbutyrate metabolite analysis were collected at |
| metabolite analysis | Baseline and during any hyperammonemic episode. |
| Serum tryptase and | Serum tryptase and serum complement C3 were measured at Baseline |
| serum complement | and in the event of a hypersensitivity reaction at the central |
| C3 | laboratory. Samples were obtained at 3 and 24 hours after the start of |
| | the reaction. |
| ADAs | Samples for ADAs were collected at Baseline and pre-dose at regular |
| | intervals throughout the study according to the schedules of |
| | assessments. In the event of a hypersensitivity reaction, additional |
| | ADA samples were taken at the time of the event and as clinically |
| | indicated thereafter. |
| PK | Samples for the determination of pegzilarginase levels were |
| | collected. The results were to remain blinded until completion of the |
| | study. |

Abbreviations: ALT = alanine aminotransferase; ALP = alkaline phosphatase; aPTT = activated partial thromboplastin time; AST = aspartate aminotransferase; BUN = blood urea nitrogen; IGF 1 = insulin-like growth factor 1; INR = international normalized ratio; PK = pharmacokinetic; PT = prothrombin time; RBC = red blood cell; sRBP = serum retinol binding protein; TIBC = total iron-binding capacity; WBC = white blood cell.

### Individualized Disease Management Plan (and 3-Day Diary Diet Record)

The current recommended disease management approach involves a framework of severe protein restriction and EAA supplementation and/or the use of ammonia scavengers. These facets of disease management are individualized for each patient by titration of the different components to reduce plasma arginine levels and ensure an adequate intake of protein for proper growth and development while avoiding hyperammonemia. General guidelines suggest that the diet for patients with ARG1 D will include approximately 50% of the protein from natural food sources and approximately 50% from amino acid supplements. This recommendation could be adjusted as clinically indicated to achieve the IDM goals appropriate for a specific subject.

Subjects were maintained on the IDM regimen prescribed prior to enrollment by the treating Investigator throughout the study. The prescribed IDM regimen was not permitted to be modified during the study unless clinically indicated in the opinion of the Investigator. The rationale for any change in IDM and the date and nature of any major (>±15% of Baseline) prescribed changes were recorded in the eCRF.

Subjects were required to maintain dietary protein intake levels that were consistent with their Baseline levels for the entire duration of the randomized DB AND the 8-week blinded period of the LTE period of the study. A consistent diet was defined as one in which the prescribed and consumed natural intact protein, medical food EAA, and calories were <±15% from Baseline.

Subjects and/or their caregivers were instructed to record all dietary intake by the study subject (natural food, medical food/EAAs, and EAA supplementation) for 3 consecutive days prior to all clinic visits during the screening and Baseline Period and before DB Visits 6, 12, 18, and 24. At these visits, the prescribed IDM regimen was also recorded. The date and time of the last dose of EAAs was recorded for each day of the 3 day diary diet record.

### Medical History

In addition to arginase deficiency, the most frequently reported medical history included Cognitive disorder, Hyperammonaemia, Developmental delay, and increased Alanine aminotransferase, which are common clinical findings in ARG1-D. A greater proportion of subjects in the pegzilarginase group had a medical history (i.e. prior to treatment) of Hyperammonaemia compared to subjects in the placebo group. Developmental delay and Cognitive disorder were reported in a lower proportion of subjects in the pegzilarginase group than in the placebo group (prior to treatment).

In general, most commonly used medications prior to the study were dietary supplements, essential amino acid formulas, vitamins and minerals as the standard of care for ARG1-D is a protein restricted diet. Ammonia scavengers were commonly taken to manage ammonia levels and medication to manage other disease symptoms.

Overall, all 32 subjects (100%) received concomitant medications during the study (Table 3). The most frequently used concomitant medications were the ammonia scavengers sodium benzoate and glycerol phenylbutyrate. The proportion of subjects who used either sodium benzoate or glycerol phenylbutyrate was similar between pegzilarginase group and the placebo group. Overall, almost all subjects in the pegzilarginase treated group (95.2%, N=20) and in the placebo group (81.8%, N=9) received ammonia scavengers during the study.

Other frequently used medications in both the pegzilarginase and placebo groups included essential amino acid formulas, baclofen, ibuprofen, paracetamol, vitamin and mineral food supplements, and cetirizine.

TABLE 3
Overview of Concomitant Medications - Preferred Names
Reported by ≥2 Subjects and Corresponding ATC Class

| | Pegzil- | | | | | |
|---|---|---|---|---|---|---|
| | arginase | | Placebo | | Overall | |
| ATC Class | (N = 21) | | (N = 11) | | (N = 32) | |
| Preferred Name | n (%) | | n (%) | | n (%) | |
| Any concomitant medications | 21 | (100) | 11 | (100) | 32 | (100) |
| Amides | 2 | (9.5) | 5 | (45.5) | 7 | (21.9) |
| Lidocaine | 2 | (9.5) | 4 | (36.4) | 6 | (18.8) |
| Amino acids and derivatives | 3 | (14.3) | 1 | (9.1) | 4 | (12.5) |
| Amino acids and derivatives | 2 | (9.5) | 0 | | 2 | (6.3) |
| Amino | 14 | (66.7) | 7 | (63.6) | 21 | (65.6) |
| acids/carbohydrates/minerals/ | | | | | | |
| vitamins, combinations | | | | | | |
| Amino acids NOS/ | 3 | (14.3) | 2 | (18.2) | 5 | (15.6) |
| carbohydrates NOS/fats | | | | | | |
| NOS/minerals NOS/ | | | | | | |
| vitamins NOS | | | | | | |
| Amino acids/carbohydrates/ | 2 | (9.5) | 1 | (9.1) | 3 | (9.4) |
| minerals/vitamins, | | | | | | |
| combinations | | | | | | |
| Cyclinex 2 | 4 | (19.0) | 2 | (18.2) | 6 | (18.8) |
| Dialamine | 2 | (9.5) | 2 | (18.2) | 4 | (12.5) |
| EAA | 6 | (28.6) | 2 | (18.2) | 8 | (25.0) |
| Aminoalkyl ethers | 4 | (19.0) | 1 | (9.1) | 5 | (15.6) |
| Diphenhydramine | 2 | (9.5) | 1 | (9.1) | 3 | (9.4) |
| Anilides | 10 | (47.6) | 3 | (27.3) | 13 | (40.6) |
| Acetaminophen | 2 | (9.5) | 1 | (9.1) | 3 | (9.4) |
| Paracetamol | 6 | (28.6) | 2 | (18.2) | 8 | (25.0) |
| Electrolyte solutions | 1 | (4.8) | 2 | (18.2) | 3 | (9.4) |
| Sodium chloride | 0 | | 2 | (18.2) | 2 | (6.3) |
| Fat/carbohydrates/ | 1 | (4.8) | 2 | (18.2) | 3 | (9.4) |
| proteins/minerals/vitamins, | | | | | | |
| combinations | | | | | | |
| Amino acids NOS/ | 1 | (4.8) | 2 | (18.2) | 3 | (9.4) |
| carbohydrates NOS/fats | | | | | | |
| NOS/minerals NOS/proteins | | | | | | |
| NOS/vitamins NOS | | | | | | |
| General nutrients | 7 | (33.3) | 1 | (9.1) | 8 | (25.0) |
| General nutrients | 3 | (14.3) | 0 | | 3 | (9.4) |
| Nutrients NOS | 3 | (14.3) | 1 | (9.1) | 4 | (12.5) |
| H2-receptor antagonists | 2 | (9.5) | 3 | (27.3) | 5 | (15.6) |
| Famotidine | 0 | | 3 | (27.3) | 3 | (9.4) |
| Pepcid [famotidine] | 2 | (9.5) | 0 | | 2 | (6.3) |
| Influenza vaccines | 5 | (23.8) | 2 | (18.2) | 7 | (21.9) |
| Flu | 3 | (14.3) | 0 | | 3 | (9.4) |
| Multivitamins with minerals | 6 | (28.6) | 3 | (27.3) | 9 | (28.1) |
| Minerals NOS/vitamins NOS | 6 | (28.6) | 2 | (18.2) | 8 | (25.0) |
| Other antiepileptics | 5 | (23.8) | 3 | (27.3) | 8 | (25.0) |
| Keppra [levetiracetam] | 3 | (14.3) | 1 | (9.1) | 4 | (12.5) |
| Other antihistamines for | 3 | (14.3) | 1 | (9.1) | 4 | (12.5) |
| systemic use | | | | | | |
| Desloratadine | 2 | (9.5) | 1 | (9.1) | 3 | (9.4) |
| Other centrally acting agents | 4 | (19.0) | 3 | (27.3) | 7 | (21.9) |
| Baclofen | 4 | (19.0) | 3 | (27.3) | 7 | (21.9) |
| Other combinations of | 3 | (14.3) | 2 | (18.2) | 5 | (15.6) |
| nutrients | | | | | | |
| Creatine | 3 | (14.3) | 0 | | 3 | (9.4) |
| Other combinations of | 0 | | 2 | (18.2) | 2 | (6.3) |
| nutrients | | | | | | |
| Other viral vaccines | 6 | (28.6) | 4 | (36.4) | 10 | (31.3) |
| Covid-19 vaccine | 2 | (9.5) | 2 | (18.2) | 4 | (12.5) |
| Pfizer BioNTech Covid-19 | 1 | (4.8) | 2 | (18.2) | 3 | (9.4) |
| vaccine | | | | | | |
| Sars-Cov-2 vaccine | 2 | (9.5) | 0 | | 2 | (6.3) |
| Penicillins with extended | 2 | (9.5) | 3 | (27.3) | 5 | (15.6) |
| spectrum | | | | | | |
| Amoxicillin | 1 | (4.8) | 3 | (27.3) | 4 | (12.5) |
| Piperazine derivatives | 7 | (33.3) | 6 | (54.5) | 13 | (40.6) |
| Cetirizine | 6 | (28.6) | 5 | (45.5) | 11 | (34.4) |
| Propionic acid derivatives | 7 | (33.3) | 3 | (27.3) | 10 | (31.3) |
| Ibuprofen | 4 | (19.0) | 3 | (27.3) | 7 | (21.9) |
| Proton pump inhibitors | 1 | (4.8) | 5 | (45.5) | 6 | (18.8) |
| Lansoprazole | 1 | (4.8) | 2 | (18.2) | 3 | (9.4) |
| Omeprazole | 0 | | 2 | (18.2) | 2 | (6.3) |
| Second-generation | 0 | | 2 | (18.2) | 2 | (6.3) |
| cephalosporins | | | | | | |
| Cefoxitin | 0 | | 2 | (18.2) | 2 | (6.3) |
| Serotonin (5-HT3) antagonists | 0 | | 4 | (36.4) | 4 | (12.5) |
| Ondansetron | 0 | | 3 | (27.3) | 3 | (9.4) |
| Solutions for parenteral | 5 | (23.8) | 5 | (45.5) | 10 | (31.3) |
| nutrition | | | | | | |
| Dextrose | 2 | (9.5) | 2 | (18.2) | 4 | (12.5) |
| Glucose | 2 | (9.5) | 1 | (9.1) | 3 | (9.4) |
| SMOFlipid | 2 | (9.5) | 1 | (9.1) | 3 | (9.4) |
| Solutions for parenteral | 2 | (9.5) | 1 | (9.1) | 3 | (9.4) |
| nutrition | | | | | | |
| Substituted alkylamines | 3 | (14.3) | 2 | (18.2) | 5 | (15.6) |
| Chlorphenamine | 3 | (14.3) | 2 | (18.2) | 5 | (15.6) |
| Various alimentary tract and | 20 | (95.2) | 9 | (81.8) | 29 | (90.6) |
| metabolism products | | | | | | |
| Buphenyl | 2 | (9.5) | 1 | (9.1) | 3 | (9.4) |
| Pheburane | 3 | (14.3) | 0 | | 3 | (9.4) |
| Ravicti | 10 | (47.6) | 5 | (45.5) | 15 | (46.9) |
| Sodium benzoate | 10 | (47.6) | 5 | (45.5) | 15 | (46.9) |
| Sodium phenylbutyrate | 2 | (9.5) | 2 | (18.2) | 4 | (12.5) |
| Vitamin D and analogues | 10 | (47.6) | 3 | (27.3) | 13 | (40.6) |
| Colecalciferol | 3 | (14.3) | 1 | (9.1) | 4 | (12.5) |
| Uvedose | 3 | (14.3) | 1 | (9.1) | 4 | (12.5) |

### 1.6 Efficacy Evaluation

The study is comprised of 2 periods, the placebo-controlled Double-blind period and the LTE period.

### Primary Endpoint

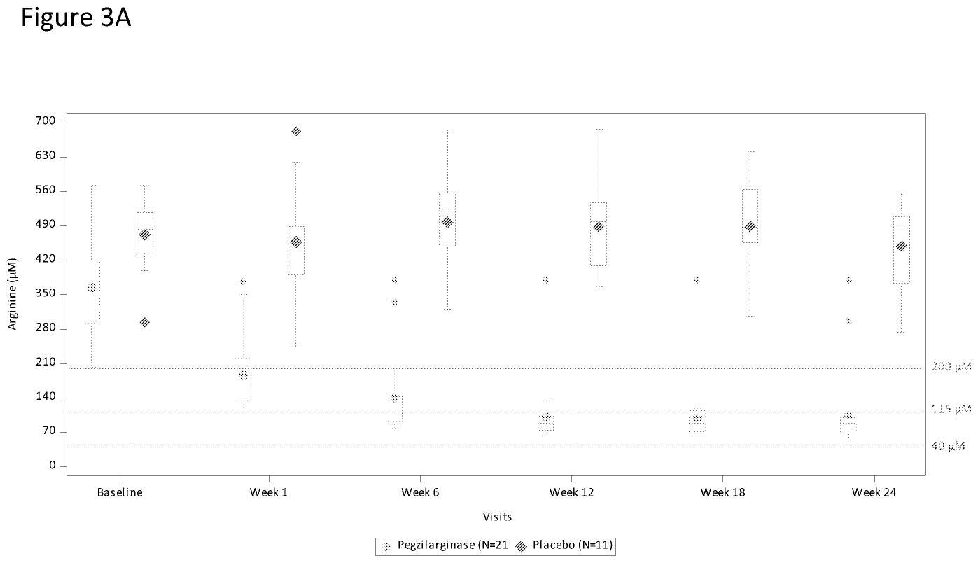

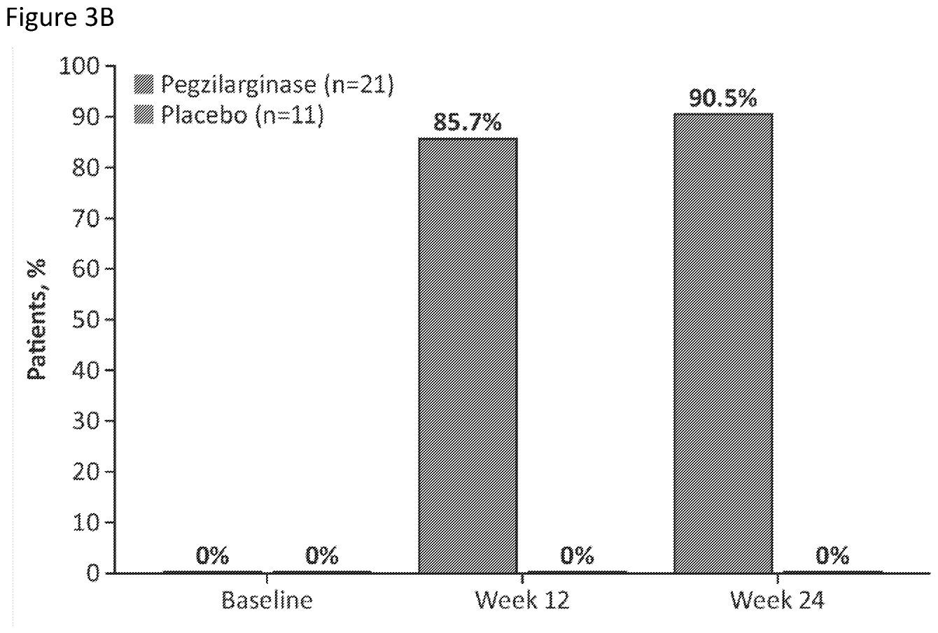

Subjects treated with pegzilarginase had a rapid and sustained decrease in mean plasma arginine, with declines seen at Week 1 and by Week 12 were within normal limits, which was sustained through Week 24. There was little change in mean plasma arginine in the placebo group (FIG. 3A). At Week 24, the mean plasma arginine in the pegzilarginase group was 105.49 μM, compared to the mean plasma arginine in the placebo group of 448.82 μM. Treatment with pegzilarginase resulted in meaningful decreases in arginine (i.e., values <200 μM) compared to placebo starting at Week 6 and were maintained through Week 24 of treatment.

### Primary Endpoint Analysis

The primary analysis used mixed effect model with repeated measures (MMRM) method. Log-transformed plasma arginine data were used in the analysis. One subject dropped out prior to Week 24; the corresponding missed Week 24 data were imputed using the last observation carried forward (LOCF) approach. At Baseline, the geometric mean (SD) plasma arginine levels were lower in the pegzilarginase group (354.0 μM [SD 1.30 μM]) than in the placebo group (464.7 μM [SD 1.21 μM]). Treatment with pegzilarginase resulted in significant reductions (p<0.0001) in plasma arginine compared to placebo starting at Week 6 and were maintained through Week 24 of treatment. At Week 24, pegzilarginase demonstrated a 76.7% reduction in mean plasma arginine compared to placebo.

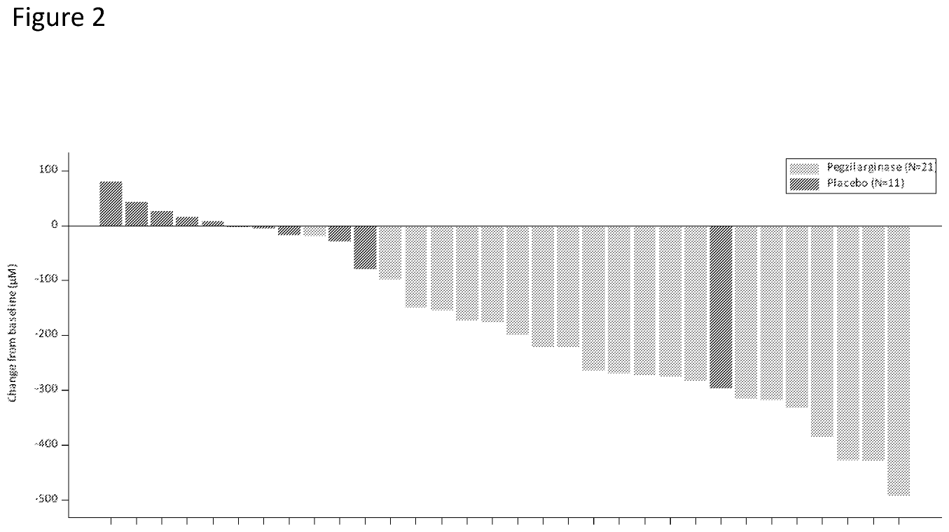

In assessing the reduction in plasma arginine by individual subject, nearly all subjects in the pegzilarginase group (95%, N=20) had a meaningful change in plasma arginine at Week 24 compared to the placebo group (FIG. 2).

### Key Secondary Endpoints

a. Two Minute Walk Test

The Two Minute Walk Test (2MWT) is a shorter modification of the 6-minute version (e.g., National Institutes of Health [NIH] Toolbox motor domain-2MWT [Bohannon 2014]). The standardized methodology as defined by the American Thoracic Society (2002) was followed. Results of the 2MWT were recorded as distance completed in meters and percent change from Baseline.

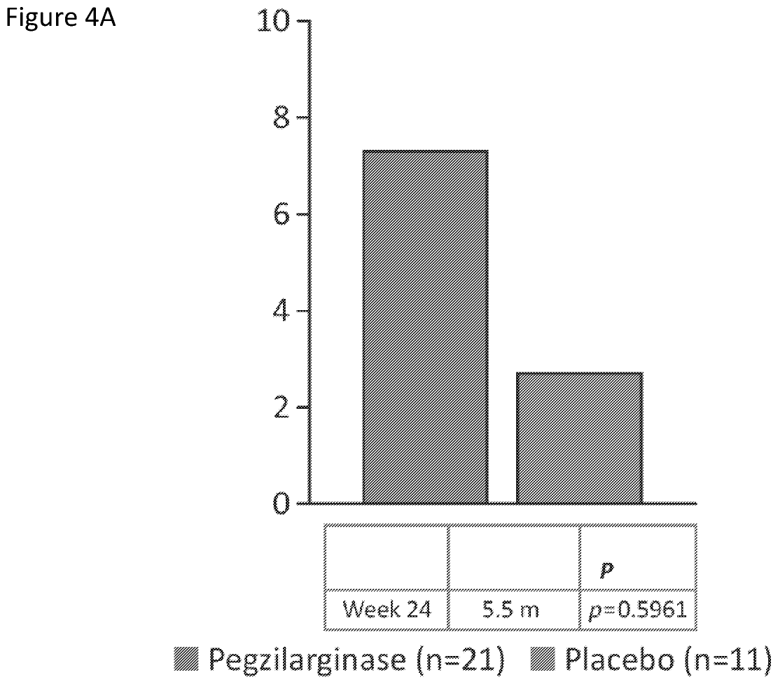

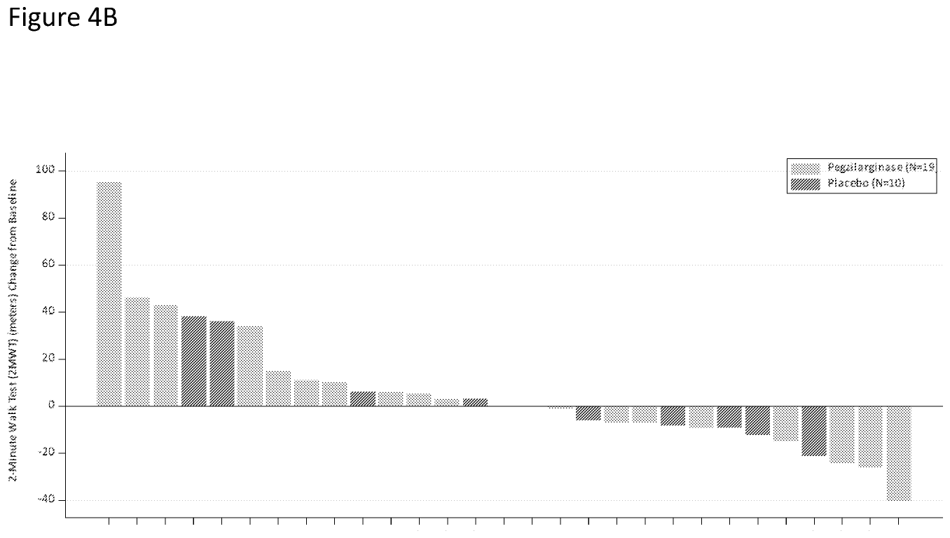

Subjects treated with pegzilarginase demonstrated an improvement in the 2MWT compared to the placebo group. At Baseline, the mean (SD) distance walked over 2 minutes for subjects who received pegzilarginase was longer at 109.0 meters (SD 55.76 meters) compared to subjects who received placebo at 99.9 meters (SD 38.61 meters). At Week 24, the mean (SD) distance walked over 2 minutes in the pegzilarginase group was 115.9 meters (SD 51.81), representing a 7.3 meter increase from Baseline, compared to the mean distance walked in the placebo group of 102.3 meters (SD 51.10), representing a 2.7-meter difference from Baseline. Although the change from Baseline to Week 24 for the LS mean difference in 2MWT between the pegzilarginase and placebo groups (5.5 meters) did not meet statistical significance (p=0.5961) (FIG. 4A), it showed that subjects in the pegzilarginase group had a larger improvement than subjects in the placebo group (FIGS. 4A and 4B).

In assessing changes from Baseline in the 2MWT by individual subject with available Baseline and Week 24 data (N=29), a greater proportion of subjects in the pegzilarginase group (52.6%, N=10) had improvements in the change from Baseline in the 2MWT distance compared to the placebo group (40%, N=4) and the magnitude of improvement was generally larger by subjects in the pegzilarginase group versus the placebo group.

b. Gross Motor Function Measure-88 Part E

The Gross Motor Function Measure-88 (GMFM-88, also referred to as GMFM) is a clinical measure designed to evaluate gross motor function by observing the subject's ability to initiate and complete certain movements. For this study, only Dimensions D and E were assessed. A scoring system was used by a trained observer for each item of the GMFM covering 2 dimensions: standing (Part D) and walking, running, and jumping (Part E).

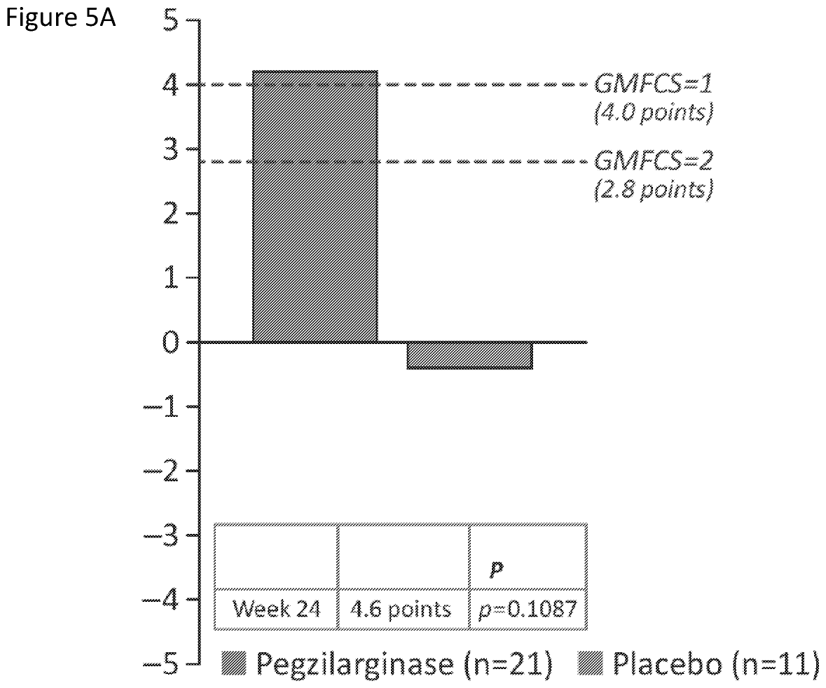

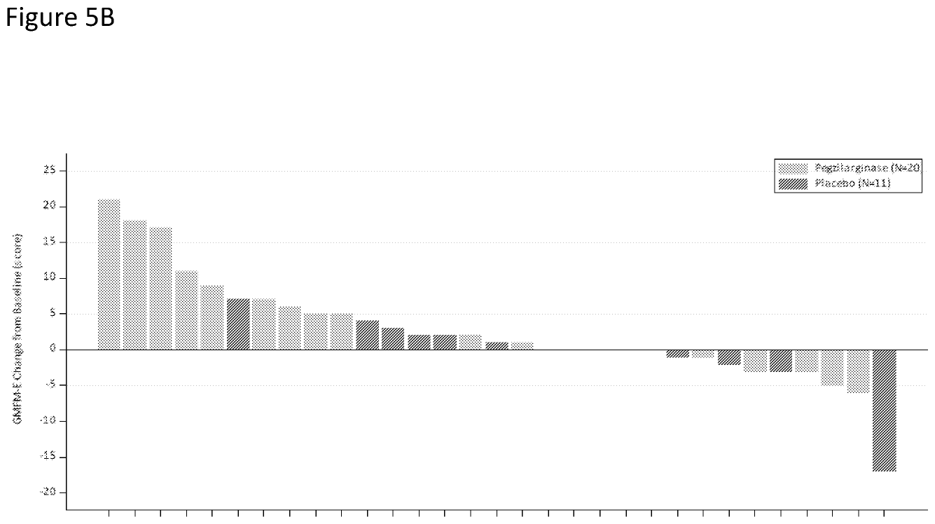

Subjects treated with pegzilarginase demonstrated an improvement in the GMFM-E compared to the placebo group. At Baseline, the mean (SD) GMFM-E score for subjects in the pegzilarginase group was (48.3 [SD 19.93]) and (46.5 [SD 24.56]) in the placebo group. At Week 24, the mean (SD) in (SD) GMFM-E score in the pegzilarginase group was 52.0 (SD 21.27), representing a 4.2-point increase from Baseline-compared to the mean (SD) score of 46.71 (SD 25.71) in the placebo group representing a 0.4 (51.10) decrease from Baseline. The change from Baseline to Week 24 for the LS mean difference in GMFM-E score between the pegzilarginase and placebo groups was 4.6 points, representing a trend in improvement (p=0.1087) (FIG. 5A).

Individual responses are summarized in the waterfall plot of GMFM-E and change from Baseline at Week 24 (FIG. 5B). A similar proportion of subjects in the pegzilarginase group (55.0%, N=11) had improvements in change from Baseline in GMFM-E compared to the placebo group (54.5%, N=6), however, the magnitude of improvement was generally larger in the pegzilarginase subjects compared to placebo subjects (FIG. 5B).

In assessing changes from Baseline in the GMFM-E by individual subject, a similar proportion of subjects in the pegzilarginase group (55.0%, N=11/20) had improvements in change from Baseline at Week 24 in GMFM-E score compared to the placebo group (54.5%, N=6/10), however, the magnitude of improvement was generally larger by subject in the pegzilarginase group versus the placebo group.

c. Responders Based on Arginine Values Less than 200 μM (<200 μM)

Subjects treated with pegzilarginase demonstrated an improvement in the plasma arginine levels meeting treatment guidelines compared to the placebo group. Current guidelines recommend a treatment goal of plasma arginine below 200 μM. The proportion of subjects able to achieve this goal (responders) was assessed by treatment group. Nineteen of 21 subjects (90.5%) in the pegzilarginase group were responders after 24 weeks of treatment, having achieved arginine values <200 μM compared to none in the placebo group (Table 4).

TABLE 4
Proportion of Responders with Arginine Values <200
μM During the Double-blind period

| | |
|---|---|
| Pegzilarginase | Placebo |
| (N = 21) | (N = 11) |
| n (%) | n (%) |

| | | | |
|---|---|---|---|
| Week 12 | | | |
| Arginine Value <200 | | | |
| μM at Timepoint | | | |
| Responders | 20 | (95.2) | 0 |
| Non-responders | 1 | (4.8) | 11 (100) |

| | | |
|---|---|---|
| p-value - Fisher's | <0.0001 | — |

| | | | |
|---|---|---|---|
| Exact Test^{a} | | | |
| Week 24 | | | |
| Arginine Value <200 | | | |
| μM at Timepoint | | | |
| Responders | 19 | (90.5) | 0 |
| Non-responders | 2^{b} | (9.5) | 11 (100) |

| | | |
|---|---|---|
| p-value - Fisher's | <0.0001 | — |

Exact Test^{a}

d. Responders Based on Arginine Values within the Normal Range (40 to 115 μM)

Subjects in the pegzilarginase group exhibited an improvement in the plasma arginine levels resulting in normal arginine levels compared to the placebo group. While current guidelines recommend plasma arginine below 200 μM, achieving values in the normal range is an important therapeutic goal. The proportion of subjects able to achieve this treatment goal was assessed by treatment group. At Week 24, 90.5% of subjects in the pegzilarginase group met this criteria for response, having achieved arginine values within the normal range, and none of the subjects in the placebo group met this criteria for response (Table 5 and FIG. 3B).

TABLE 5
Proportion of Responders with Arginine Value within Normal
Range During the Double-blind period (40 to 115 μM)

| | |
|---|---|
| Pegzilarginase | Placebo |
| (N = 21) | (N = 11) |
| n (%) | n (%) |

| | | | |
|---|---|---|---|
| Arginine value 40 to | | | |
| 115 μM at Week 12 | | | |
| Responders | 18 | (85.7) | 0 |
| Non-responders | 3 | (14.3) | 11 (100) |

| | |
|---|---|
| p-value - Fisher's | <0.0001 |

| | | | |
|---|---|---|---|
| Exact Test^{a} | | | |
| Arginine value 40 to | | | |
| 115 μM at Week 24 | | | |
| Responders | 19 | (90.5) | 0 |
| Non-responders | 2 | (9.5) | 11 (100) |

| | |
|---|---|
| p-value - Fisher's | <0.0001 |

Exact Test^{a}

e. Changes in Ornithine and Guanidino Compounds at Week 24

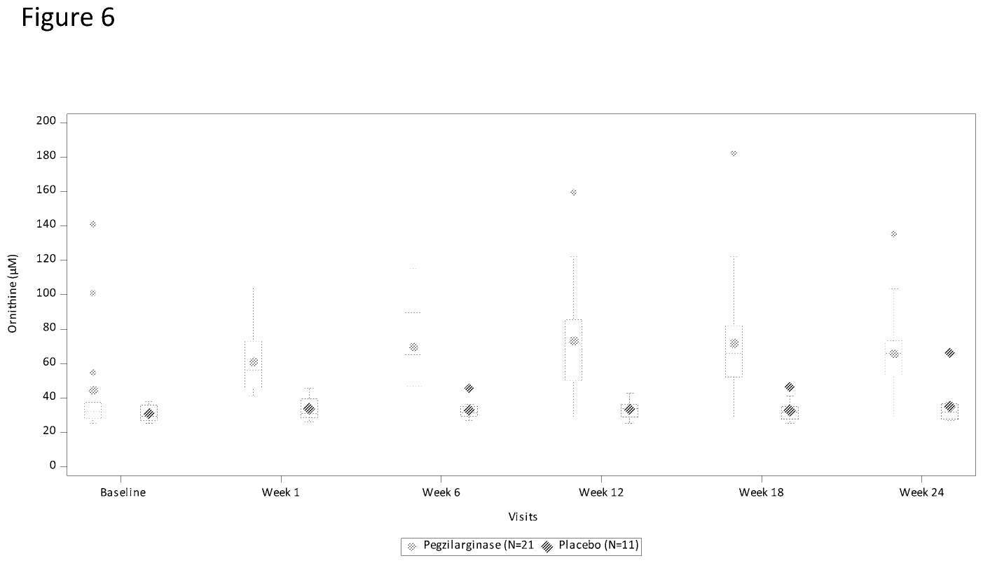

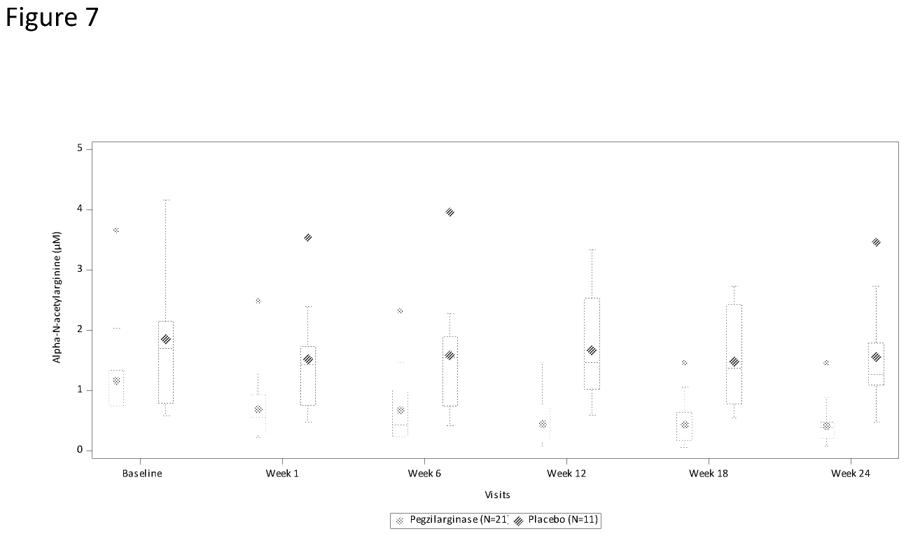

Baseline ornithine values and change from Baseline are presented in FIG. 6, and Baseline guanidino compound values and change from Baseline are presented in FIGS. 7-10.

Ornithine and urea are products of the hydrolysis of arginine by arginase I in the final step of the urea cycle. Ornithine levels are generally low in subjects with ARG1-D due to the lack of the enzyme arginase I available to convert it from arginine. A summary of ornithine change from Baseline is presented in FIG. 6.

Subjects treated with pegzilarginase had a rapid and sustained increase in mean plasma ornithine, with increases seen at Week 1, continued improvement through Week 12 levels, which was sustained through Week 24. At Baseline, the median ornithine level in the pegzilarginase group was 32.1 μM and in the placebo group was 29.2 μM. Subjects treated with pegzilarginase had increases in ornithine at Week 1 that continued to increase with further dosing compared to minimal change in the placebo group (FIG. 6). Treatment with pegzilarginase resulted in significant increases (p<0.0001) in ornithine compared to placebo starting at Week 6 and were maintained through Week 24 of treatment. At Week 24, the mean ornithine level in the pegzilarginase group was 67.7 μM, demonstrated a 106.9% increase in mean ornithine compared to placebo. Starting at Week 6 and maintained through Week 24 of treatment, the mean ornithine level in the pegzilarginase group was at least 60 μM.

f. Guanidino Compounds in Arginase-1 Deficiency

Guanidino compounds (GC) are direct and indirect products of arginine metabolism, and are generally elevated in ARG1-D. These compounds may contribute to the pathophysiological changes in addition to hyperargininemia in ARG1-D, such as seizures and hepatic dysfunction, although exact mechanisms are not yet fully understood (Deshmukh 1991; Franzoi 2018; Yu 2013).

Abnormalities in guanidino compounds, nitric oxide, and homoarginine concentrations have been implicated to play a pathophysiological role in Arg-1 D. Elevation of guanidino compounds has been observed in animals and humans affected by hyperargininaemia. These result from elevated arginine levels via the arginine: glycine amidinotransferase reaction. Guanidino compounds like guanidinoacetate have been shown to be epileptogenic and may be responsible for neurological impairment and epilepsy in these patients.

At Baseline, plasma levels of guanidine compounds were similar between treatment arms, although values of alpha-keto δ-guanidinovaleric acid and alpha N-acetylarginine were incrementally higher in the pegzilarginase group. The median values of arginic acid, guanidinoacetic acid, alpha-keto-δ-guanidinovaleric acid, and alpha N-acetylarginine in pegzilarginase group were 2.5 μM, 3.1 μM, 4.6 μM, and 1.1 μM, respectively, in the pegzilarginase group, and 2.8 μM, 3.5 μM, 5.4 μM, and 1.7 μM, respectively, in the placebo group.

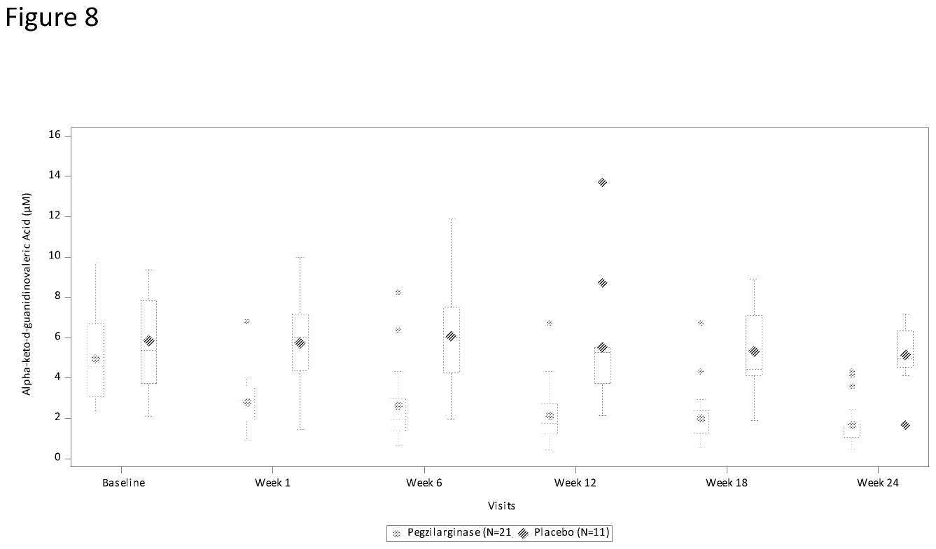

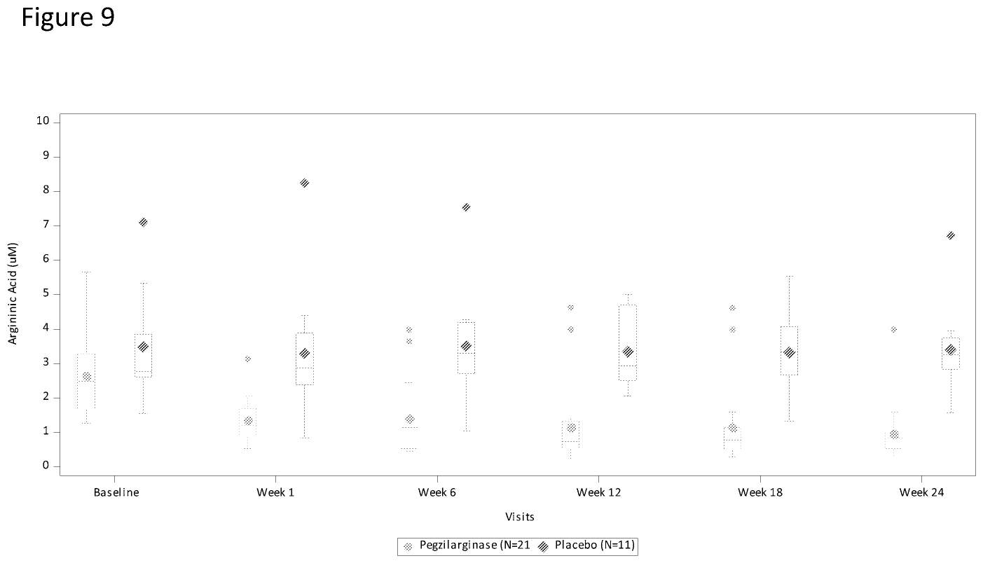

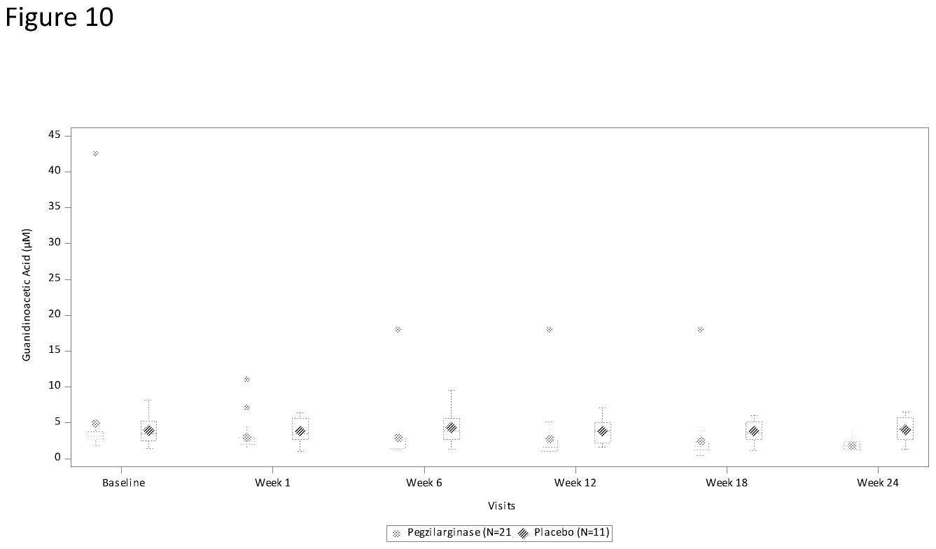

For the pegzilarginase group, decreases in the levels of all four GCs were observed starting at Week 1 and maintained over time through Week 24 of the Double-blind period (FIG. 7, FIG. 8, FIG. 9, FIG. 10, respectively). Whereas for the placebo group, levels of all four GCs fluctuated over time but remained similar to Baseline levels through Week 24. Treatment with pegzilarginase resulted in significant decreases (p value range: 0.0059 to <0.0001) in GCs compared to placebo starting at Week 6 and were maintained through Week 24 of treatment. At Week 24, pegzilarginase demonstrated a 50% to 70% reduction in GCs (compared to placebo) and was associated with reduced levels of plasma arginine. Starting at Week 6 and maintained through Week 24 of treatment, the mean alpha N-acetylarginine level in the pegzilarginase group was decreased to less than 0.5 μM (0.3 μM at Week 24); the mean alpha-keto-δ-guanidinovaleric acid level in the pegzilarginase group was decreased to less than 2 μM (1.3 μM at Week 24; the mean argininic acid level in the pegzilarginase group was decreased to less than 1 μM (0.7 μM at Week 24); and the mean guanidinoacetic acid level in the pegzilarginase group was decreased to less than 2 μM (1.7 μM at Week 24).

g. Guanidino Compounds in Renal Damage

When disease causes kidney damage toxic guanidino compounds can accumulate in the patient. Arginine produced from citrulline originates mostly from kidneys. Arginine is involved in guanidino compound biosynthesis, which requires inter-organ co-operation. In renal insufficiency, citrulline can accumulate in the plasma in proportion to renal damage. Thus, disturbances in arginine and guanidino compound metabolism are expected in several tissues.

Levillain etal used a model of nephrectomy based on ligating branches of the renal artery allowing measurement arginine and guanidino compound metabolism simultaneously in injured (left) and healthy (right) kidneys.

The metabolism and/or tissue content of several guanidino compounds were disturbed. The experimentally proven toxins α-keto-δ-guanidinovaleric acid and guanidinosuccinic acid (GSA) accumulated in the injured kidney. Nephrectomy also affected the guanidino compound levels and metabolism in muscles and liver. Thus, disturbances in renal metabolism and function (ie kidney injury) can lead to accumulation of toxic guanidino compounds.

There is also evidence that when the neurotoxic substance guanidinoacetate accumulates above normal levels it causes seizures. Similarly, reduction guanidinoacetate levels in patient plasma has been associated with reduction in the epileptogenic effects of GAA. There is also evidence that guanidinoacetate plays a pathogenetic role in the development of extrapyramidal movement disorder.

It has long been recognized that uraemic toxins are likely to contribute to CNS injury and neurodegeneration either directly or indirectly (Arnold etal).

Patients with chronic kidney disease (CKD) are frequently afflicted with neurological complications. Uremic encephalopathy is a cerebral dysfunction caused by the accumulation of toxins resulting from acute or chronic renal failure. Guanidino compounds have long been implicated in uremic encephalopathy such as guanidinosuccinic acid (GSA), methylguanidine, guanidine and creatinine which have been shown to be elevated in serum, brain and cerebrospinal fluid of uraemic patients. These compounds are known to induce convulsions in the experimental setting.

In regard to diagnosis and management of CKD, EEG findings can be of diagnostic value as the degree of EEG changes correlate with severity of encephalopathy. The typical features of an EEG in uraemic neuropathy are often non-specific such as a slowing of the alpha rhythm with excess delta and theta waves. The presence of triphasic sharp waves on EEG is considered a specific feature of metabolic encephalopathies. Triphasic waves are typically seen in uraemic g.

h. Other Disorders

Secondary GAA increases are demonstrated in argininemia, S-adenosylhomocysteine hydrolase deficiency (SAHH deficiency), and cobalamin disorders. Arginine supplementation is also known to contribute to mild GAA increases.encephalopathy.

i. Guanidino Compounds in Guanidinoacetate Methyltransferase (GAMT) Deficiency

Guanidinoacetate methyltransferase (GAMT) deficiency (MIM 601240) is an autosomal recessive inborn error of creatine synthesis, biochemically reflecting creatine deficiency and a marked accumulation of guanidinoacetate (GAA) in brain and body fluids, which results in physical or mental disabilities, such as global developmental delay/intellectual disability (DD/ID) epilepsy, movement disorders, speech or language delay, and behavioral problems. Affected individuals exhibit marked impairment of expressive speech, autistic features, and varying neurological manifestations, including epilepsy and movement disorders. GAA in affected patients is believed to be neurotoxic.

Strategies to reduce guanidinoacetate (GAA) levels include substrate deprivation via an arginine restricted diet as well as competitive inhibition of arginine glycine amidinotransferase (AGAT) activity via high-dose L-ornithine supplementation. AGAT is the enzyme responsible for GAA synthesis. Although medical diets aiming to reduce arginine intake and lower systemic arginine availability have been shown to be effective in lowering GAA levels and ameliorating disease manifestations associated with high GAA levels, the required diets are problematic for a number of reasons.

j. Guanidino Compounds in S-Adenosylhomocysteine Hydrolase Deficiency

The AHCY gene encodes S-adenosylhomocysteine hydrolase (EC 3.3.1.1), which catalyzes the hydrolysis of S-adenosylhomocysteine to adenosine and homocysteine. Guanidinoacetate is observed to be elevated in S-adenosylhomocysteine hydrolase deficiency.

k. Changes in Gross Motor Function Measure Part D (GMFM-D)

At Baseline, the mean (SD) GMFM-D score was 28.0 points (SD 9.61) for subjects in the pegzilarginase group and 29.5 points (SD 12.42) for subjects in the placebo group. At Week 24, the mean (SD) GMFM-D score was 30.5 points (SD 10.09) in the pegzilarginase group and 28.2 points (SD 13.28) in the placebo group. The change demonstrated a numerical improvement in subjects in the pegzilarginase group.

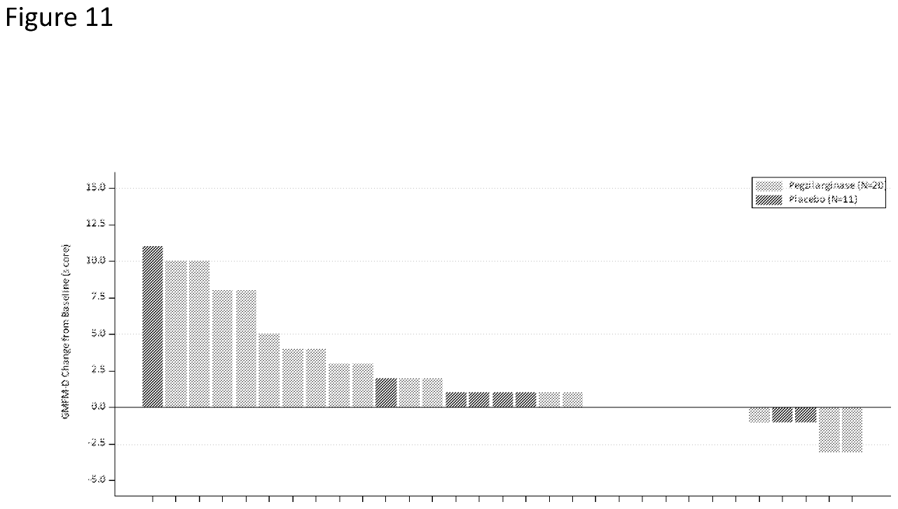

Individual responses are summarized in the waterfall plot of GMFM-D and change from Baseline at Week 24 (FIG. 11). A greater proportion of subjects in the pegzilarginase group (65.0%, N=13) had improvements in the mean change from Baseline at Week 24 in the GMFM D score compared to the placebo group (54.5%, N=6), and the magnitude of improvement was generally larger in the pegzilarginase subjects versus the placebo subjects.

l. Arginine and Clinical Outcomes Individual and Composite Response

Considering normalization of arginine levels and the composite of functional assessments after 24 weeks of treatment, within the evaluable subjects, the response at the subject level and in aggregate, demonstrated a differential response in favor of subjects treated with pegzilarginase compared to the placebo.

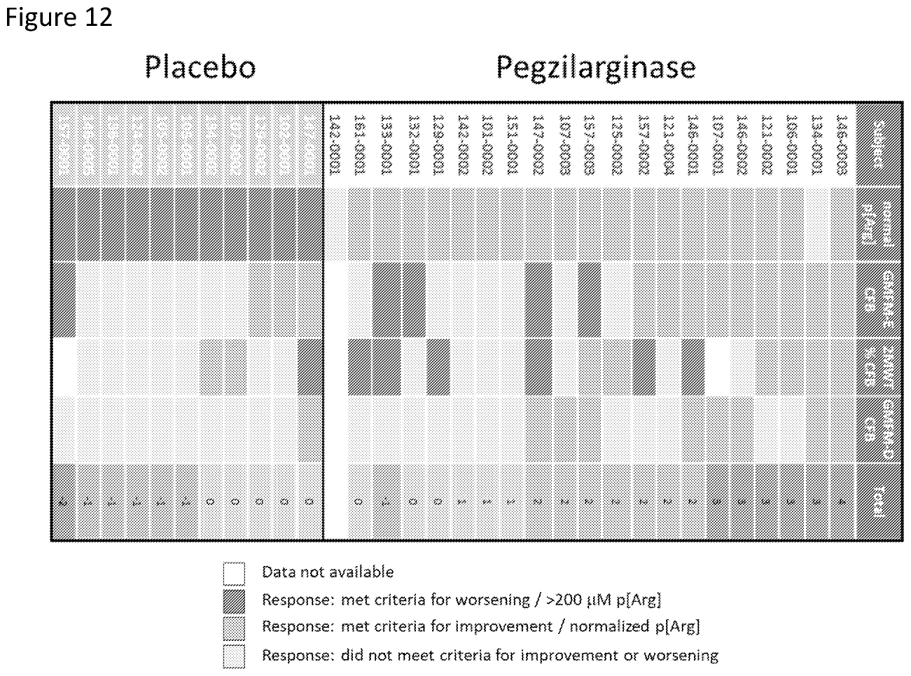

A heat map was developed to represent subjects who met the threshold for normalization of arginine levels as well as met the criteria for response in the 2MWT, GMFM-E, and GMFM-D to characterize relationship between reduction in arginine levels and clinical outcomes (FIG. 12). In the pegzilarginase treated group 13 of the 17 subjects with normalized arginine levels who were evaluable for response had one or more assessments meeting the criteria for a meaningful clinical improvement whereas no subject in the placebo groups had a normalized arginine level with a corresponding clinical improvement, although 4 of 9 evaluable subjects did have clinically meaningful improvements across 1 assessment domain. There were 8 and 6 subjects that met >2 criteria for clinical response irrespective of worsening, and without worsening, respectively, in the pegzilarginase group and 0 subjects in the placebo group.

m. Improvement of Spasticity and Other Measures of Neurological/Neuromotor Manifestations Using the Modified Ashworth Scale

The Modified Ashworth Scale (MAS) was developed to assess the spasticity of subjects with central nervous system lesions and is used to measure the resistance to passive movement about a joint due to spasticity. The scale utilizes a scoring scale of 0 (no spasticity) to 4 (total rigidity). Thus, a decrease in MAS score indicates improvement in spasticity. The mean MAS lower body score was defined as the average of the values for all muscle groups which have a score value >0.

At Baseline, the mean (SD) MAS lower body scores were 0.77 (SD 1.076) in the pegzilarginase group and 0.25 (SD 0.433) in the placebo group. By Week 24, there was a numeric improvement in the pegzilarginase group compared to placebo. The mean change from Baseline in the MAS lower body scores for subjects in the pegzilarginase group and the placebo group was −0.16 (SD 0.519) and −0.03 (SD 0.056), respectively. These improved scores indicate that 4 of 5 subjects with Baseline spasticity in the pegzilarginase group demonstrated improvements while only 1 of 2 subjects showed improvement in placebo group.

n. Nutritional Management of Patients

Clinical work with Urea Cycle Disorders (UCDs) has revealed considerable variation in protein tolerance, with age, sex, and genotype all contributing to this variability. Patients with the mildest forms of a UCD may tolerate a normal diet, in contrast to patients with a severe form, who will require a very restricted diet. The aim of treatment for the patient with a UCD is to maintain normal plasma ammonia and amino acid concentrations with nutritional management and drug therapy. Nutritional therapy for a UCD consists of a protein-restricted diet supplemented with an EAA mixture to ensure adequate intakes of essential and conditionally essential amino acids. Enough protein-free energy must be administered both to permit the normal turnover of protein necessary at all ages and to prevent net catabolism of body proteins.

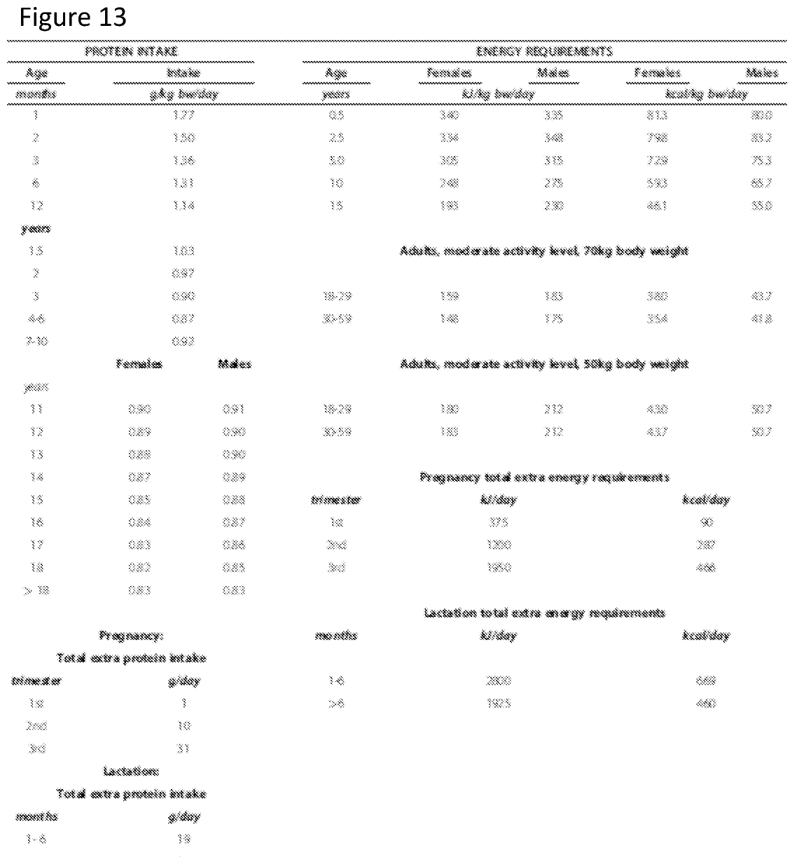

Long-term management seeks to minimize nitrogen load on the urea cycle. The amount of natural protein tolerated by each patient must be individually determined and by titration against ammonia. The WHO Food and Agriculture Organization have set ‘safe levels of protein intake’ calculated as an age adjusted mean which can be used as a guide. Lower protein intakes may still be adequate but individualized over-restriction may compromise growth and cause metabolic instability. If the intake is too low, additional supplementation may be indicated. An adequate energy supply must also be guaranteed to prevent catabolism and consequent hyperammonemia. The FAO/WHO/UNU 2007 Report (summarized in FIG. 13 which is from Harberle et al) can be used as a guide to energy intakes. Patients with reduced mobility will have lower energy expenditures and therefore lower energy requirements.

Essential amino acids (also known as indispensable amino acids) are amino acids that humans and other vertebrates cannot synthesize from metabolic intermediates. These amino acids must be supplied from an exogenous diet because the human body lacks the metabolic pathways required to synthesize these amino acids. Although variations are possible depending on the metabolic state of an individual there are nine essential amino acids: phenylalanine, valine, tryptophan, threonine, isoleucine, methionine, histidine, leucine, and lysine. These essential amino acids can be obtained from a single naturally occurring protein source such as animal based sources of nutrition. The non-essential, also known as dispensable amino acids, can be excluded from a diet. The human body can synthesize these amino acids using only the essential amino acids.

Supplementation of EAAs and other essential nutrients is required when natural protein tolerance is too low to achieve normal growth and metabolic stability. Currently, there is no method to increase natural protein tolerance.

A typical supplementation approach for UCDs is to provide 20-30% of the total protein intake as EAA supplements. However, protein restriction for ARGD1 patients is likely more severe and supplements of EAA may be up to 50% of total protein. EAA's can be given as an equally divided dose with 3 or 4 main meals to enhance utilization and prevent nitrogen overload.

Deviations from a standard healthy adult's metabolic state may place the body in a metabolic state that requires more than the standard-essential amino acids to be nitrogen balance. Infants and children may also require more than the standard-essential amino acids. In general, the optimal ratio of essential amino acids and nonessential amino acids requires a balance dependent on physiological needs that differs between individuals (including different disease states).

While there is individual variation the World Health Organization estimates a mean total protein requirement of 0.66 g/kg per day, intakes of about 0.18 g/kg per day of indispensable amino acids and 0.48 g/kg per day of dispensable amino acids.

Protein aversion is a common feature of UCDs. Neonatal symptoms of UCD usually present after the first 24-48 hours of life in infants born at term after a normal pregnancy and delivery. Common presenting symptoms are poor feeding, vomiting, lethargy, hypotonia, respiratory distress, irritability, and seizures. Patients have been reported in whom a detailed history obtained after presentation with severe neurologic symptoms revealed severe, previously unrecognized protein aversion. Many children experience a period of fussy eating behavior during the first few years of life. Children with UCD present with protein aversion and are especially fussy about meat, eggs, and dairy products but do like fruit and vegetables, in contrast to most “normal” fussy children, who tend to have more problems with vegetables. Metabolic deterioration in patients may occur due to non-compliance with the proscribed diet. It has been suggested that patients might eat more protein than recommended because of a reluctance to give up favorite foods, poor acceptance by their families, or severe peer pressure.

o. Diet Record

Prior to enrollment in the study, disease management in all 32 subjects included treatment with dietary protein restriction and amino acid supplementation, consistent with current guidelines for ARG1-D management. Sites were instructed to minimize changes to within 15% of Baseline for subjects' dietary protein intake to keep the diet stable as much as possible throughout the study.

Median total prescribed protein (including natural and EAA protein) at Baseline were similar between treatment groups, while median total prescribed calories was lower in the pegzilarginase group versus the placebo group. Median total consumed calories and total consumed protein (including natural and EAA protein) at Baseline were lower in the pegzilarginase group than in the placebo group.

The change from Baseline for median total prescribed EAA protein and total consumed EAA protein from Week 12 to Week 24 was 0.0 for both.

The median change from Baseline in total prescribed natural protein was 0.0 grams for both treatment groups at Weeks 12 and 24. Small fluctuations in the change from Baseline for median total consumed natural protein from Week 12 and Week 24 were observed in the pegzilarginase group (−0.2 and −0.5, respectively) and in the placebo group (0.1 and 0.7, respectively).

p. Diet Management Plan (and 3-Day Diary Diet Record)

Overall, most subjects from the pegzilarginase group had an increase of >15% or remained within 15% of their total consumed calories, total consumed protein/day, and total consumed natural protein. For total consumed EAA protein, the majority of subjects remained within 15% of Baseline. However, by Week 24, a higher proportion of subjects increased their total protein consumption by more than 15% in the pegzilarginase group compared to placebo (42.9% [9 subjects] versus 18.2% [2 subjects]) (Table 6). Despite a higher proportion of subjects with higher total protein intake the subjects had normalized arginine, hence, the dietary fluctuations did not negatively impact pegzilarginase's ability to control arginine.

TABLE 6
Summary Diet Management

| | Pegzilarginase | | Placebo | | Total | |
|---|---|---|---|---|---|---|
| Protein Measure | (N = 21) | | (N = 11) | | (N = 32) | |
| Week | n (%) | | n (%) | | n (%) | |
| Total Consumed Calories | | | | | | |
| Week 12 | | | | | | |
| Increase of >15% | 7 | (33.3) | 2 | (18.2) | 9 | (28.1) |
| Within +/−15% | 7 | (33.3) | 6 | (54.6) | 13 | (40.6) |
| Decrease of >15% | 1 | (4.8) | 1 | (9.1) | 2 | (6.3) |
| Week 24 | | | | | | |
| Increase of >15% | 8 | (38.1) | 2 | (18.2) | 10 | (31.3) |
| Within +/−15% | 8 | (38.0 | 6 | (54.5) | 14 | (43.8) |
| Decrease of >15% | 2 | (9.5) | 1 | (9.1) | 3 | (9.4) |
| Total Consumed EAA | | | | | | |
| Protein/Day (g) | | | | | | |
| Week 12 | | | | | | |
| Increase of >15% | 0 | | 0 | | 0 | |
| Within +/−15% | 13 | (61.9) | 8 | (72.7) | 21 | (65.6) |
| Decrease of >15% | 1 | (4.8) | 1 | (9.1) | 2 | (6.3) |
| Week 24 | | | | | | |
| Increase of >15% | 2 | (9.5) | 1 | (9.1) | 3 | (9.4) |
| Within +/−15% | 10 | (47.6) | 7 | (63.6) | 17 | (53.1) |
| Decrease of >15% | 3 | (14.3) | 1 | (9.1) | 4 | (12.5) |
| Total Consumed Natural | | | | | | |
| Protein/Day (g) | | | | | | |
| Week 12 | | | | | | |
| Increase of >15% | 6 | (28.6) | 4 | (36.4) | 10 | (31.3) |
| Within +/−15% | 5 | (23.3) | 4 | (36.4) | 9 | (28.1) |
| Decrease of >15% | 4 | (19.0) | 1 | (9.1) | 5 | (15.6) |
| Week 24 | | | | | | |
| Increase of >15% | 8 | (38.1) | 4 | (36.4) | 12 | (37.5) |
| Within +/−15% | 8 | (38.1) | 5 | (45.5) | 13 | (40.6) |
| Decrease of >15% | 2 | (9.5) | 0 | | 2 | (6.3) |
| Total Consumed | | | | | | |
| Protein/Day (g) | | | | | | |
| Week 12 | | | | | | |
| Increase of >15% | 6 | (28.6) | 3 | (27.3) | 9 | (28.1) |
| Within +/−15% | 6 | (28.5) | 6 | (54.6) | 12 | (37.5) |
| Decrease of >15% | 3 | (14.3) | 0 | | 3 | (9.4) |
| Week 24 | | | | | | |
| Increase of >15% | 9 | (42.9) | 2 | (18.2) | 11 | (34.4) |
| Within +/−15% | 7 | (33.3) | 7 | (63.7) | 14 | (43.8) |

| | | | | | |
|---|---|---|---|---|---|
| Decrease of >15% | 2 | (9.5) | 0 | 2 | (6.3) |

Note:
N in the headers represents the total number of subjects in the respective treatment group for the Full Analysis Set. Percentages are based on the total number of subjects in each respective treatment groups.

Unlike fats and carbohydrates, protein is not stored in the body but rather exists in a balanced state of anabolism (formation) and catabolismobreakdown). While hyperammonemic crises are less common in patients with arginase deficiency than other urea cycle defects they can still occur throughout infancy and adulthood. Current treatment of ARG1-D focuses on attempting to lower plasma arginine levels through lifelong dietary protein restriction. Protein intake is limited to the minimum required to maintain protein biosynthesis and growth.

Typically, half or more of dietary protein is given in the form of an arginine-free essential amino acid (EAA) mixture. A typical physician-recommend protein intake for a healthy person is approximately 10-53 grams per day whereas recommended intake for an arginase 1 deficiency patient is approximately 4-40 grams per day (˜25-60% less protein). The challenges of adhering to a protein-restricted diet rigorous enough to lower plasma arginine below goal levels place a significant burden on patients and their families. Adherence to this type of diet may be difficult due to previously established eating habits; could exacerbate eating disorders; and regular follow-ups are required to assess disease status.

q. Electroencephalograms

There was not a statistically significant difference between treatment groups as a whole in EEG results but individual patients did show improvements. At Baseline, 28.6% (6 subjects) from the pegzilarginase group and 45.5% (5 subjects) from the placebo group had abnormal not clinically significant EEGs, except for 1 subject. One subject from the pegzilarginase group had abnormally clinically significant EEG results at Baseline. This subject experienced generalized epileptiform discharges. No subject in the placebo group had an abnormal clinically significant EEG.

At Week 24, there were 23.8% (5 subjects) from the pegzilarginase group and 63.6% (7 subjects) from the placebo group who had abnormal EEGs but not clinically significant results. Three subjects from the pegzilarginase group who had (not clinically significant) Baseline abnormal EEGs shifted to normal EEGs at Week 24; all subjects from the placebo group who had not clinically significant Baseline abnormal EEG results had abnormal not clinically significant EEG results at Week 24. One subject from the pegzilarginase group and 2 subjects from the placebo group had normal EEG results at Baseline and then had abnormal not clinically significant EEG results at Week 24. One subject from the pegzilarginase group had an abnormal clinically significant EBG, which was noted as a pathological awake-sleep EBG. There were no subjects in the placebo group who had an abnormal clinically significant EBG.

r. Electroencephalograms and Guanidino Compounds

Electroencephalogram (EEG) Patients that had a change in EBG status (with description of guanidino compound changes) are shown in Table 7 below.

TABLE 7

| | | EEG | | NAArg | | GVA | | AA | | GAA | |
|---|---|---|---|---|---|---|---|---|---|---|---|
| | | Week | Week | Week | Week | Week | Week | Week | Week | Week | Week |
| Subject | Treatment | 0 | 24 | 0 | 24 | 0 | 24 | 0 | 24 | 0 | 24 |
| 101- | Pegz | Abnormal | Normal | 0.51- | 0.10- | 7.21- | 0.96- | 3.39- | 0.62- | 3.43- | 0.61- |
| 0002 | | NCS | | 0.87(H) | 0.14(N) | 9.1(H) | 1.91(H) | 4.17(H) | 0.87(H) | 4.13(H) | 2.12(N) |
| 107- | Pegz | Abnormal | Normal | 0.12(N)- | BLQ- | 2.24- | 0.13- | 1.06- | 0.11- | 1.68- | BLQ- |
| 0001 | | NCS | | 2.77(H) | 0.14(N) | 4.57(H) | 1.41(H) | 2.27(H) | 0.36(H) | 2.03(N) | 0.68(N) |
| 125- | Pegz | Abnormal | Normal | 0.90- | 0.13- | 3.27- | 0.45- | 4.69- | 0.32- | 3.35- | 0.70- |
| 0002 | | NCS | | 0.99(H) | 0.23(N) | 5.64(H) | 1.19(H) | 6.80(H) | 0.71(H) | 3.95(H) | 2.29(N) |
| 129- | Pegz | Abnormal | Abnrmal | 1.4- | 0.25(N)- | 3.47- | 0.65- | 2.11- | 0.38- | 2.10- | 0.58- |
| 0001 | | CS | NCS | 1.92(H) | 0.53(H) | 5.57(H) | 1.05(H) | 2.81(H) | 0.61(H) | 2.62(N) | 0.63(N) |
| 147- | Pegz | Normal | Abnormal | 0.12(N)- | 0.04- | 1.62- | 0.33- | 1.27- | 0.20- | 3.05- | 0.81- |
| 0002 | | | NCS | 0.34(H) | 0.11(N) | 3.16(H) | 0.73(H) | 2.70(H) | 0.46(H) | 4.36(H) | 2.56(N) |
| 151- | Pegz | Normal | Abnormal | 1.00- | 0.13(N)- | 4.30- | 1.49- | 2.39- | 0.55- | 3.37- | 0.33(L)- |
| 0001 | | | CS | 1.70(H) | 0.35(H) | 9.82(H) | 3.13(H) | 3.54(H) | 1.43(H) | 3.87(H) | 1.27(N) |
| 124- | Placebo | Normal | Abnormal | 0.44- | 0.74- | 0.81- | 3.80- | 0.67- | 2.36- | 1.06(N)- | 4.51- |
| 0002 | | | NCS | 2.27(H) | 1.81(H) | 4.04(H) | 7.60(H) | 3.19(H) | 3.48(H) | 5.8(H) | 6.34(H) |
| 157- | Placebo | Normal | Abnormal | 0.84- | 1.00- | 3.72- | 3.04- | 2.35- | 1.22- | 2.87(N)- | 2.51(N)- |
| 0001 | | | NCS | 1.24(H) | 1.34(H) | 4.47(H) | 5.87(H) | 3.09(H) | 3.58(H) | 3.19(H) | 3.60(H) |

* Normal Range: Guanidinoacetic acid (0.4 to 3.0 μM), a-keto-d-guanidinovaleric acid (ULN <0.05 μM), a-N-acetylarginine (0.025 to 0.255 μM), argininic acid (0.025 to 0.1 μM);
Abbreviations: H = Above Reference Range; L = Below Reference Range.

### 1.7 Patient Summaries

101-0002: At screening (week 0) the EEG was abnormal not clinically significant (NCS). After week 24 of treatment with Pegzilarginase the EEG was normal. During screening the patient had levels of α-N-acetylarginine (NAArg), argininic acid (AA), and guanidinoacetic acid (GAA) that were above the normal range. At week 24 of treatment with Pegzilarginase, GAA and NAArg had reduced into the normal range. AA was still considered high but had reduced from 3.39-4.17 μM to 0.62-0.87 μM. GVA remained high from screening (week 0) through week 24 of treatment with Pegzilarginase but had reduced from 7.21-9.1 μM to 0.96-1.91 μM.
107-0001: At screening (week 0) the EEG was abnormal not clinically significant (NCS). After week 24 of treatment with Pegzilarginase the EEG was normal. At screening this patient had NAArg of 0.12 (Normal) −2.77 (High) whereas at week 24 it was BLQ-0.14 μM (Normal). The AA remained above the normal range at screening and week 24 of treatment but had reduced from 1.06-2.27 to 0.11-0.36 μM. GAA was in the normal range throughout (although one measurement was BLQ at week 24). GVA was above the normal range throughout but had reduced from 2.24-4.57 at screening to 0.13-1.41 μM at week 24.
125-0002: At screening (week 0) the EEG was abnormal not clinically significant (NCS). After week 24 of treatment with Pegzilarginase the EEG was normal. At screening the patient had NAArg, GVA, AA, and GAA levels above the normal range. At week 24 of treatment, NAArg and GAA were within the normal range. AA remained above the normal range but had reduced from 4.69-6.80 μM to 0.32-0.71 μM. GVA remained above the normal range but had reduced from 3.27-5.64 at screening to 0.45-1.19 at week 24.
129-0001: At screening the EEG was abnormal clinically significant (CS) whereas after week 24 of Pegzilarginase treatment the EEG was abnormal not clinically significant (NCS). At screening GAA was within the normal range which was maintained at week 24 of treatment. At screening the levels of NAArg, GVA and AA were above the normal range. At week 24 of treatment, AA was above the normal range but had reduced from 2.11-2.81 μM to 0.38-0.61 μM. At week 24 of treatment, NAArg was between the normal and high range having reduced from 1.4-1.92 (H) uM to 0.25 (N)-0.53 (H) uM. At week 24 of treatment, GVA was above the normal range but had reduced from 3.47-5.57 (H) to 0.65-1.05 (H).
147-0002: At screening the EEG was normal whereas at week 24 of Pegzilarginase treatment the EEG was abnormal not clinically significant (NCS). At screening NAArg ranged from 0.12 (N) to 0.34 (H). At week 24 of treatment NAArg was within the normal range. At screening the level of GAA was above the normal range whereas at week 24 of treatment GAA was in the normal range. At screening AA was above the normal range and remained so at week 24 although it had reduced from 1.27-2.7 μM at screening to 0.2-0.46 μM at week 24 of treatment. GVA remained above the normal range although it had reduced from 1.62-3.16 at screening to 0.33-0.73 at week 24 of treatment.
151-0001: At screening the EEG was normal whereas at week 24 of treatment with Pegzilarginase the EEG was abnormal clinically significant (CS). At screening NAArg was 1.00-1.70 (above the normal range). At week 24 of treatment the NAArg level was between normal and high (0.13 (N)-0.35 (H)). At screening AA was above the upper limit of normal and remained above normal after week 24 of treatment although it had reduced from 2.39-3.54 to 0.55-1.43. At screening GAA was above the upper limit of normal at 3.37-3.87. At week 24 of treatment GAA was reduced to 0.33 (L)-1.27 (N). GVA was above normal levels but had reduced from 4.30-9.82 at screening to 1.49-3.13 at week 24 of treatment.
124-0002: At screening the EEG was normal whereas at week 24 of receiving placebo it was abnormal NCS. At screening NAArg was above the upper limit of normal and remained above throughout the placebo period (0.44-2.27 at week 0 and 0.74-1.81 at week 24). AA was above the normal at screening and remained above throughout the placebo period (0.67-3.19 at week 0 and 2.36-3.48 at week 24). GVA was 0.81-4.04 at screening and 3.80-7.60 at week 24 ie remained above the normal range. At screening GAA was 1.06 (N)-5.80 (H) ie between normal and high levels. At week 24 GAA was 4.51 (H)-6.34 (H) ie above the normal range.
157-0001: At screening the EEG was normal whereas at week 24 of placebo it was abnormal NCS. At screening NAArg, GVA, and AA were above the upper limit of normal and remained above at week 24 of the placebo treatment. At screening GAA was 2.63 (N)-3.19 (H) and at week 24 was 2.51 (N)-3.6 (H).

### 1.8 Pharmacokinetic Analysis

Adequate PK exposure was obtained with IV administration within the dose range of 0.05 to 0.2 mg/kg, which was consistent with the dose-response relationship observed in previous studies. The median time to maximum observed pegzilarginase concentration (tₘₐₓ) generally occurred rapidly (≤4.7 hours after the start of infusion), as expected following a nominal 0.5 h QW IV infusion of pegzilarginase. The pegzilarginase PK exposures (Cₘₐₓ and AUC₀₋₁₆₈) after IV administration increased in an approximately dose-proportional manner across the dose range of 0.05 to 0.2 mg/kg at steady state after repeat QW dosing (Week 12 and Week 24). The mean tin was approximately 40 hours (range: 37.3 to 43 hours), which was similar across doses and whether a single dose or at steady state. Steady state was achieved on or before Week 12 based on the available data and sampling time; however, it is theoretically expected to be reached after 2 weeks of consistent QW dosing based on tin. Exposures were relatively constant for Week 12 and Week 24 versus Week 1, with negligible accumulation after weekly dosing.

### 1.9 Pharmacodynamic Analysis

The rapid onset of action tin of pegzilarginase resulted in early, consistent, and sustained reductions in plasma arginine. At all timepoints evaluated post dosing (Week 1, Week 12, and Week 24), arginine remained below the upper limit of normal (115 μM) for the majority of the QW dosing interval. At Week 12 and 24, plasma arginine levels at post dose were generally lower than baseline values for all subjects and all doses, and below the guideline recommended level (<200 μM) for all but 1 subject. Excursions below 40 μM are observed early after IV dosing, with the majority of subjects approaching normal range by 96 hours and all subjects having levels above 40 μM at the 168 hours timepoint.

### 1.10 Efficacy Conclusions

### Double-Blind Period

The objective of this study was to demonstrate the efficacy of pegzilarginase relative to placebo based on a statistically significant decrease in plasma arginine concentrations and neuromotor assessments. The rapid onset and prolonged ti, of pegzilarginase resulted in early, consistent, and sustained reductions in plasma arginine levels through Week 24 of the placebo-controlled Double-blind period and improvements in mobility.

At Baseline, the mean plasma arginine was 365.44 μM [SD 93.682] in the pegzilarginase group and 471.74 [SD 79.928] uM in the placebo group. Following treatment with pegzilarginase after 24 weeks, mean plasma arginine values in the pegzilarginase group demonstrated early, consistent, and sustained reductions.

Plasma arginine levels were reduced by 76.7% compared to placebo (p<0.0001), both to within the treatment guidelines (<200 μM) and to normal levels.

At Week 12 and Week 24 nearly all subjects on pegzilarginase achieved plasma arginine values <200 μM (95.2% [20/21] and 90.5% [19/21], p<0.0001).

At Week 12 and Week 24 nearly all subjects achieved plasma arginine values within the normal range (40 to 115 μM) (85.7% [18/21] and 90.5% [19/21], p<0.0001).

The mean improvement from Baseline in functional mobility as measured by GMFM E was 4.2 (N=20) at Week 24 for the pegzilarginase group compared to a 0.4 decrease in the placebo group. The LS mean difference at Week 24 was 4.6 (p=0.1087), demonstrating a clinically meaningful trend in improvement. This improvement exceeds the minimum clinically important differences for patients with analogous neuromotor diseases, which are applicable to ARG1-D based on psychometric analysis in patients with this disorder.

An increase from Baseline in the mean distance walked over 2 minutes as measured by the 2MWT was demonstrated at Week 24 in the Double-blind period. For the Double-blind period, the mean improvement from Baseline at Week 24 was 7.3 meters (N=19) in subjects treated with pegzilarginase compared to 2.3 meters (N=10) in the placebo group. The LS mean difference at Week 24 between the pegzilarginase group and the placebo group was 5.5 meters, representing a numerical improvement (p=0.5961).

Subjects with GMFCS classifications of >II demonstrated greater improvements in the GMFM-E and 2MWT compared to subjects with GMFCS I when treated with pegzilarginase compared to placebo.

Increases in ornithine and approximately 50 to 70% decreases in GCs accompanied the reduction in plasma arginine in subjects treated with pegzilarginase and no meaningful changes were noted in the placebo group.

The mean improvement from Baseline in GMFM-D was 2.5 (N=20) at Week 24 for the pegzilarginase group compared to −1.3 in the placebo group, demonstrating a numerical improvement with potential clinical relevance.

The mean (SD) improvement from Baseline in the Vineland adaptive behavior (VABS-II) composite score was 1.4 (SD 16.54) in the pegzilarginase group and −1.6 (SD 8.78) in the placebo group at Week 24. The LS mean difference at Week 24 was 4.9, indicating numerical improvement in favor of pegzilarginase.

At Week 24, the proportion of eligible subjects (N=17 for pegzilarginase and N=9 for placebo) meeting the criteria for clinical response was higher for each individual component (2MWT, GMFM-D, GMFM-E) in the pegzilarginase compared to the placebo group.
2MWT, N=7 subjects met criteria:
   Pegzilarginase group: 29.4% (N=5)
   Placebo group: 22% (N=2)
GMFM-E, N=11 subjects met criteria:
   Pegzilarginase group: 52.9% (N=9)
   Placebo group: 18.2% (N=2)
GMFM-D, N=7 subjects met criteria:
   Pegzilarginase group: 41.1% (N=7)
   Placebo group: 0% (N=0)

When no worsening in any response (response criteria definition 1) in any individual component was also considered, the composite clinical response was similar in the pegzilarginase group (41.2%; N=7) compared to the placebo group (44.4%; N=4) for a response on any single endpoint.

Using the composite clinical response (response criteria definition 2 with or without worsening in a response), there was a clinically meaningful difference between pegzilarginase and placebo in favor of pegzilarginase where the number of responders was numerically higher in the pegzilarginase group (64.7%; N=11) compared to the placebo group (44.4%; N=4).

A higher percentage of subjects met the criteria for a clinically meaningful response in 2 or more assessments in the pegzilarginase group (47.0%; N=8 irrespective of worsening, 35.3% N=6 without worsening) compared to the placebo treated group, which had none. Additionally, the percentage of subjects achieving clinical response in all 3 measurements was higher in the pegzilarginase group (11.7%; N=2) compared to the placebo group, which had none. The magnitude of response was generally higher in subjects treated with pegzilarginase compared to placebo.

In the pegzilarginase group, the mean improvement from Baseline in MAS lower body scores was 0.13 (N=7) at Week 24. In the placebo group, the mean improvement from Baseline in MAS lower body scores was 0.04 (N=5) at Week 24. This difference may be clinically relevant.

No definitive differences were observed in the other endpoints (i.e., 9-Hole Pegboard Dexterity Test, Caregiver and Clinician Global impression of change, responders on VABS-II, FMS/GFAQ, PEDSQL, Neurocognitive assessments).

Following QW IV administration PK exposure increased in an approximately dose-proportional manner across the dose range evaluated, with negligible accumulation, and correlated with arginine reduction.

Anti-drug antibodies were few, transient, and resolved without meaningful clinical impact.

### Hyperammonemia Events

Overall, 21.9% of subjects (N=7) experienced Hyperammonemia episode AEs, with a smaller percentage in the pegzilarginase group (14.3% [N=3]) compared to the placebo g. Hyperammonaemia was reported in a lower percentage of subjects in the pegzilarginase group (9.5% [N=2]) compared to the placebo group (27.3% [N=3]). Hyperammonemia encephalopathy was experienced by 4.8% of subjects (N=1) in the pegzilarginase group and 9.1% of subjects (N=1) in the placebo group. No TEAEs of Hyperammonemia crisis were reported.

### Example 2: Brief Summary of PEACE

As described above, PEACE was designed to assess the effects of treatment with pegzilarginase (n=21) versus placebo (n=11) from baseline through a prespecified 24-week treatment period.

### 1.1 Topline Results

Achieved the primary endpoint with a highly statistically significant 76.7% reduction in mean plasma arginine in pegzilarginase treated patients (p<0.0001) compared to placebo.

Normal plasma arginine levels (40-115 μM) achieved in 90.5% of pegzilarginase treated patients compared to no patients in the placebo arm.

Accompanying improvements in the key secondary mobility assessments in pegzilarginase treated patients compared to patients in the placebo arm.
Gross Motor Function Measure Part E (GMFM-E): The least squares mean score improved by 4.2 units for pegzilarginase treated patients and worsened by 0.4 units in the placebo arm (p=0.1087), establishing a positive trend.
2-minute walk test (2MWT): The least squares mean distance increased 7.4 meters in pegzilarginase treated patients and 1.9 meters in the placebo arm (p=0.5961).

Pegzilarginase was well-tolerated and safety data were consistent with results from previous clinical trials. Adverse events were generally mild to moderate in severity. There were no study discontinuations due to adverse events.

### 1.2 Patient-Level Outcomes Analysis

In an analysis of individual patients that were Gross Motor Function Classification System (GMFCS) Level I-III with predefined clinical response criteria there were clinically important differences between the pegzilarginase treated patients (n=17) and placebo (n=9).
Eleven patients (65%) treated with pegzilarginase reached or exceeded prespecified response criteria for at least one mobility assessment compared to four patients (44%) receiving placebo.
Eight patients (47%) met or exceeded prespecified clinical response criteria for at least two of the mobility outcomes compared to no patients receiving placebo.

Six of the patients meeting or exceeding the clinical response threshold for at least two mobility outcomes also showed no worsening on any other mobility endpoint. Additional analysis was conducted on these six patients to compare improvement to age- and sex-matched norms (Table 8 below).

TABLE 8
Patient-Level Outcomes Analysis

| | | GMFM-E | | GMFM-D |
|---|---|---|---|---|
| Patient | | (Score Range, | | (Score Range, |
| (Age) | | 0-72 units) | 2MWT | 0-39 units) |
| 1 | (6 y) | Improved by 7 | Improved by 34 m to | Improved by 2 |
| | | Total score: 69 | 152 m | Total score: 35 |
| | | | Achieved normal | |
| | | | age/sex-matched mean | |
| 2 | (6 y) | Improved by 18 | Improved by 46 m to | Improved by 4 |
| | | Total score: 45 | 96 m | Total score: 32 |
| 3 | (12 y) | Improved by 6 | Improved by 43 m to | Improved by 5 |
| | | Total score: 72 | 167 m | Total score: 39 |
| | | (max) | | (max) |
| 6 | (14 y) | Improved by 9 | No worsening | Improved by 8 |
| | | Total score: 62 | | Total score: 37 |
| 16 | (2 y) | Improved by 11 | Improved by 44 m* to | Improved by 8 |
| | | Total score: 52 | 150 m | Total score: 32 |
| | | | Exceeded normal | |
| | | | age/sex-matched mean | |
| 17 | (3 y) | Improved by 21 | Improved by 95 m to | No worsening |
| | | Total score: 62 | 164 m | |
| | | | Exceeded normal | |
| | | | age/sex-matched mean | |

Ages are at the time of enrollment; Normalization defined at ±15% of age/sex-matched mean distance per the NIH toolbox
*Reflects change from Week 12; distance at baseline not assessed owing to age

### 1.3 Additional Secondary Efficacy Endpoints

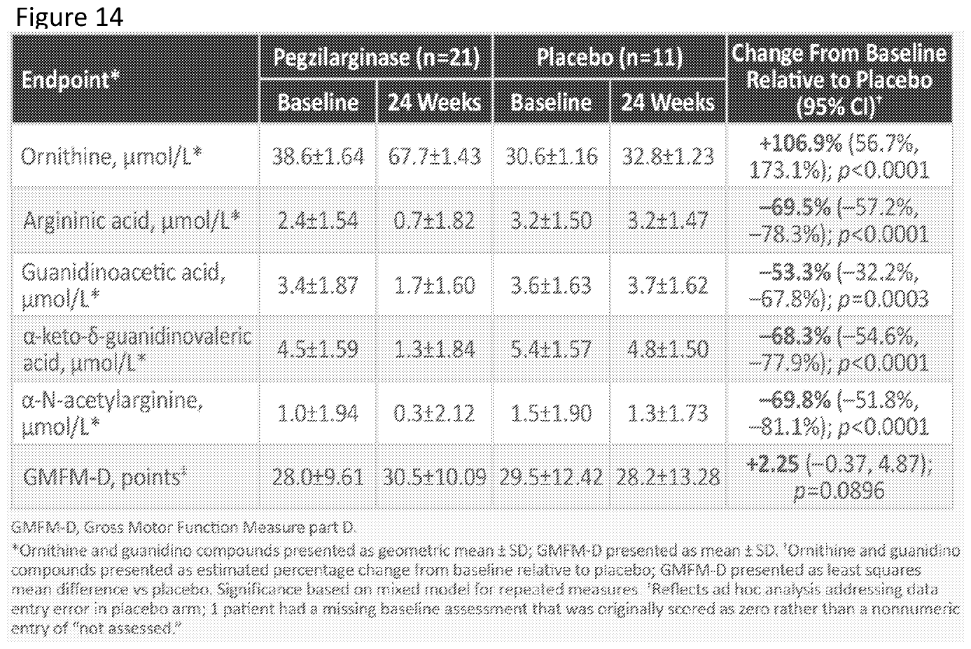

In a post hoc analysis correcting for a missed assessment that was improperly scored as 0 rather than “not assessed”, the least squares mean GMFM-D score of patients treated with pegzilarginase improved from baseline by 2.25 units compared to placebo (p=0.0896). Pegzilarginase treated patients also showed statistically significant biochemical improvements in measures of ornithine and guanidino compounds compared to placebo, consistent with pegzilarginase mechanism of action (FIG. 14).

### Example 3: Adaptive Dosing Simulations

Based upon the accumulated patient data from phase 3 dosing simulations were run to assess various dosing strategies.

The starting doses were administered for four weeks, from Week 5 onwards doses could be titrated based on the value of the 168 h post-dose L-arginine sample using the following algorithm:
If any 168 h post-dose L-arginine was >150 uM the dose was increased, with doses capped at 0.2 mg/kg.
If two consecutive 168 h post-dose L-arginine were <50 uM the dose was decreased.

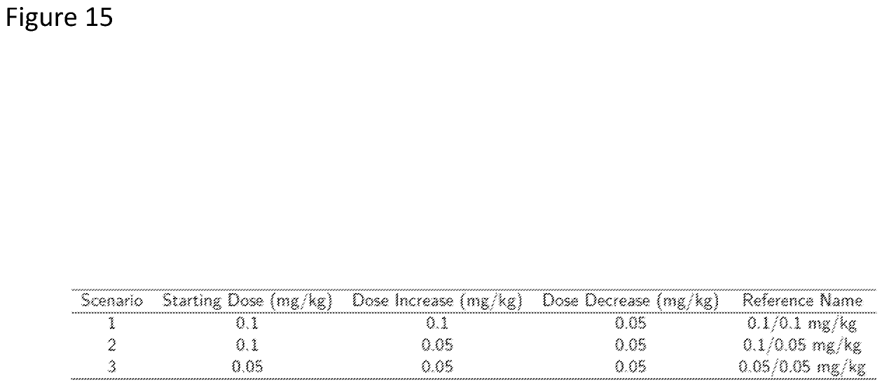

The dosing algorithm is optimized to ensure that 168 h post-dose L-arginine remains within 50-150 uM. The dosing strategies as shown in FIG. 15 were tested for IV and SC administration:

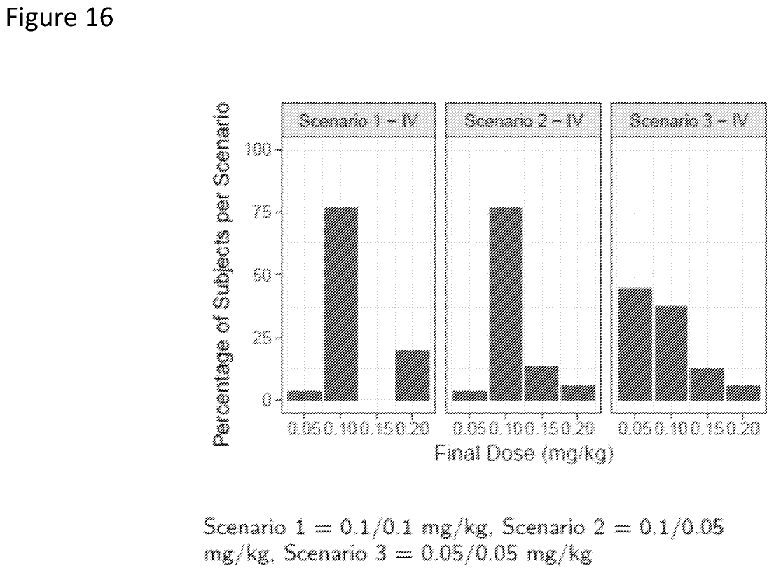

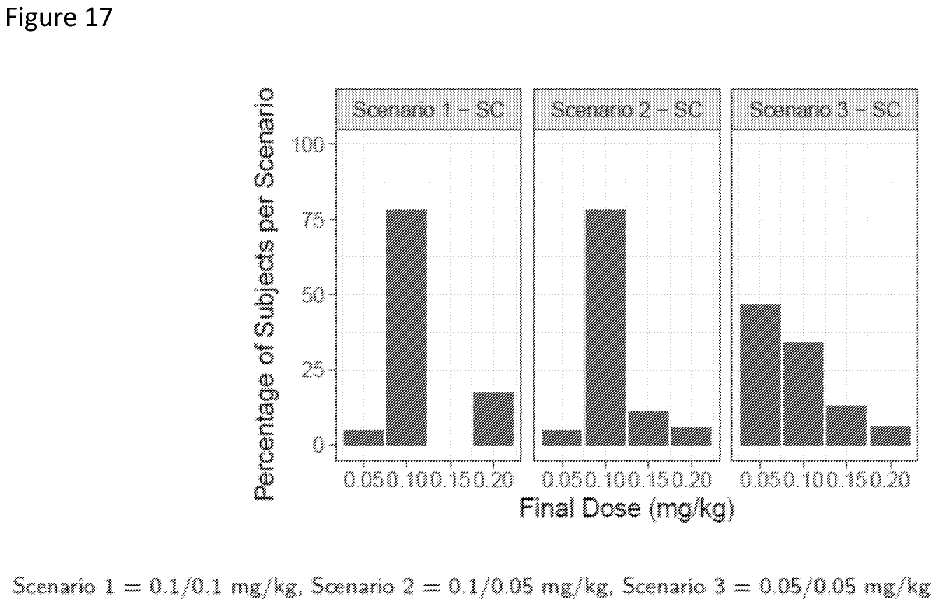

In Scenario 1 (0.1/0.1 mg/kg) for about 75% of subjects 0.1 mg/kg was sufficient to maintain L-arginine control. The starting dose of 0.05 mg/kg was sufficient to maintain L-arginine control for approximately 44% of subjects. Similar results were seen for SC dosing route. (FIG. 16 and FIG. 17)

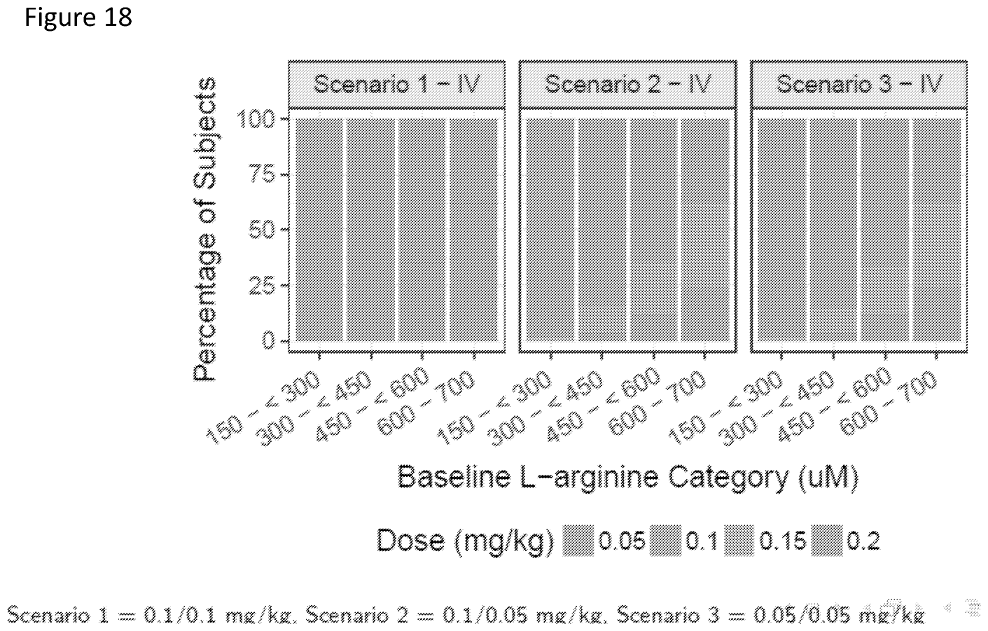

### 1.1 Relationship Between Baseline L-Arginine and Final Dose (FIG. 18)

Subjects with a higher baseline L-arginine tended to require a higher final maintenance dose regardless of dosing strategy. Similar results were seen for SC dosing route.

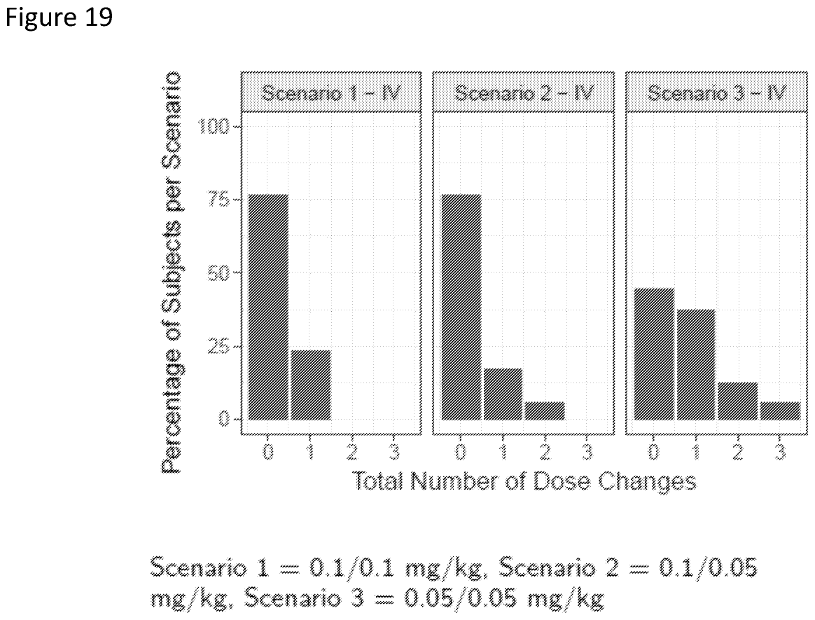

### 1.2 Number of Dose Titrations Required for IV Route (FIG. 19)

Scenario 1 (0.1/0.1 mg/kg) required the fewest number of dose titrations, while Scenario 3 (0.05/0.05 mg/kg) required the most dose titrations (this was a consequence of the starting dose and size of dose increase). Similar results were seen for the SC dosing route.

Doses of 0.05-0.2 mg/kg (IV and SC) resulted in L-arginine being <200 μM (guideline recommended level) for almost the entire dosing interval (>98%) with similar levels of control regardless of dosing strategy.

SC dosing across all dosing scenarios resulted in tighter control of L-arginine over the dosing interval compared to IV dosing, (less time <40 μM and more time in 40-115 μM). The 168 h post-dose L-arginine value correlates with the control of L-arginine during the dosing interval and can be used for monitoring and dose titration decisions.

A 168 h post-dose L-arginine of 100-200 μM appeared to minimize the time that L-arginine remained <40 μM while maximizing the time that L-arginine remained in the normal range and below the guideline recommended level.

A dose increment of 0.05 mg/kg allows for more flexibility in dosing, however requires more dose titrations.

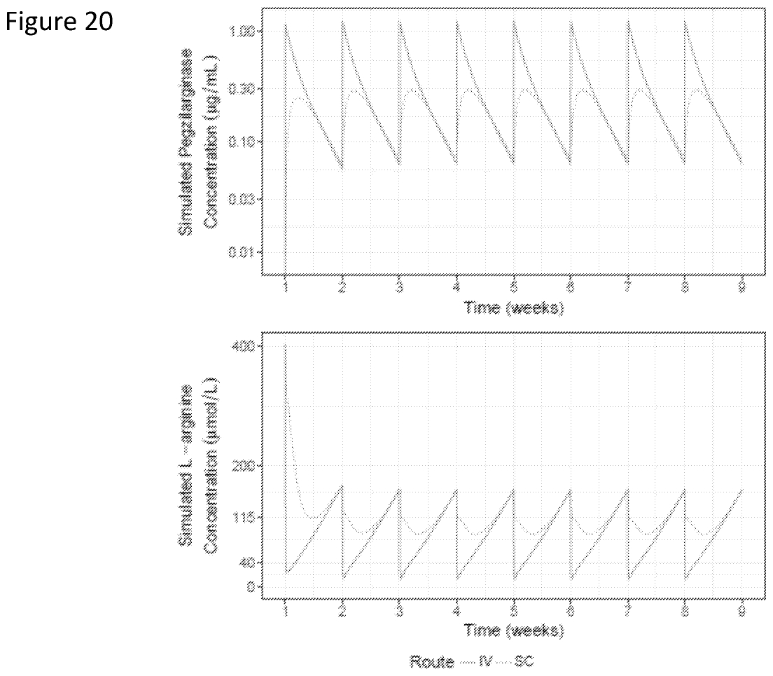

### 1.3 Differences Between IV and SC Dosing (FIG. 20)

A simulation was performed of two subjects with body weights of 31 (in the absence of any anti-drug antibodies). Eight doses of 0.05 mg/kg were administered QW. One subject received IV dosing whereas one subject received SC dosing.

SC dosing results in overall lower concentrations of pegzilarginase compared to IV dosing, as such the reduction is L-arginine is less substantial. SC dosing has less fluctuations in pegzilarginase concentrations compared to IV dosing, therefore the response is sustained for a longer period.

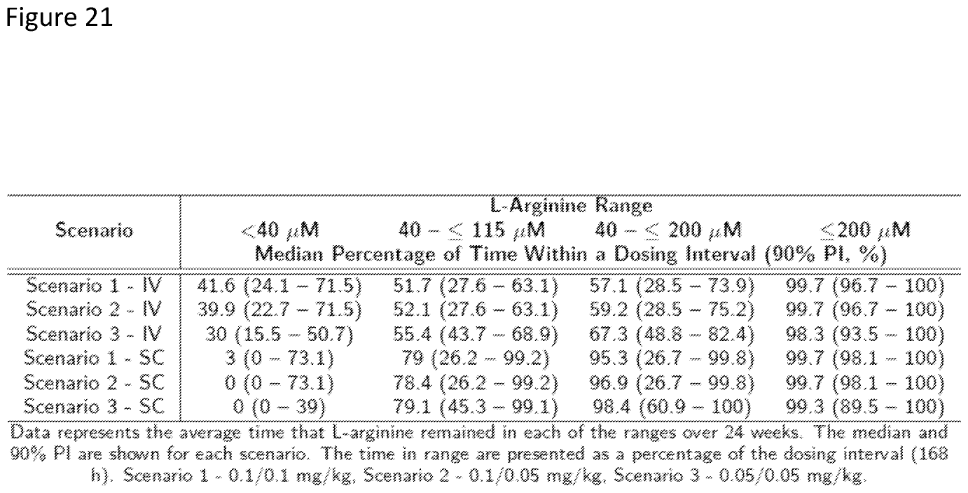

### 1.4 L-arginine Time in Recommended Range (FIG. 21)

All dosing strategies and dosing routes resulted in L-arginine remaining <200 μM for almost the entirety of the dosing interval. The SC route minimized the time that L-arginine was <40 μM compared to IV route. Similarly, the SC dosing resulted in L-arginine remaining 40-200 μM and 40-115 μM (normal range) for a longer period compared to IV dosing.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### REFERENCE

Amayreh W, Meyer U, Das A M, et al. Treatment of arginase deficiency revisited: guanidinoacetate as a therapeutic target and biomarker for therapeutic monitoring. Dev Med Child Neurol. 2014; 56 (1): 1021-1024.
American Thoracic Society. ATS Statement: Guidelines for the six-minute walk test. Am J Respir Crit Care Med. 2002; 166:111-117.
Ammann-Reiffer C, Bastianen C, Van Hedel H. Measuring change in gait performance of children with motor disorders: assessing the Functional Mobility Scale and the Gillette Functional Assessment Questionnaire walking scale. Developmental Medicine & Child Neurology: John Wiley & Sons Ltd on behalf of Mac Keith Press. Open Access Article 2018.
Andersson C, Asztalos L, Mattsson E. Six-minute walk test in adults with cerebral palsy. A study of reliability. Clin Rehabi. 2006; 20 (6): 488-95.
Bohannon R W, Bubela D, Magasi S, et al. Comparison of walking performance over the first 2 minutes and the full 6 minutes of the Six-Minute Walk Test. 2014 BMC Res Notes. 2014; 7:269. doi: 10.1186/1756-0500-7-269.
Bohannon R W, Wang Y C, Bubela D, Gershon R C. Normative two-minute walk test distances for boys and girls 3 to 17 years of age. Phys Occup Ther Pediatr. 2018; 38 (1): 39 45. doi: 10.1080/01942638.2016.1261981.
Burrage L C, Sun Q, Elsea S H, et al. Human recombinant arginase enzyme reduces plasma arginine in mouse models of arginase deficiency. Hum Mol Genet. 2015; 24 (22): 6417-6427.
Carvalho D R, Brand G D, Brum J M, Takata R I, Speck-Martins C E, Pratesi R. Analysis of novel ARG1 mutations causing hyperargininemia and correlation with arginase I activity in erythrocytes. Gene. 2012; 509:124-130. doi: 10.1016/j.gene.2012.08.003.
Cederbaum S D, Shaw K N, Spector E B, Verity M A, Snodgrass P J, Sugarman G I. Hyperargininemia with arginase deficiency. Pediatr Res. 1979; 13:827-833.
Cederbaum S D, Moedjono S J, Shaw K N, Carter M, Naylor E, Walzer M. Treatment of hyperargininaemia due to arginase deficiency with a chemically defined diet. J Inherit Metab Dis. 1982; 5:95-99.
Centers for Disease Control and Prevention (United States). CDC Growth Charts: United States (2002). Accessed from www.cdc.gov/growthcharts/cdc_charts.htm.
Cowley D M, Bowling F G, McGill J J, van Dongen J Morris D. Adult-onset arginase deficiency. J Inherit Metab Dis. 1998; 21 (6): 677-678.
De Deyn P P, Marescau B, Qureshi I A, et al. Hyperargininemia: a treatable inborn error of metabolism. In De Deyn P P, Marescau B, Qureshi I A, Mori A, Guanidino Compounds in Biology and Medicine II. London: John Libbey & Company Limited. 1997; 53-69.
Deshmukh D R, Meert K, Sarnaik A P, Marescau B, De Deyn P P. Guanidino compound metabolism in arginine-free diet induced hyperammonemia. Enzyme. 1991; 45:128-136.
Desnick R J, Schuchman E H. Enzyme replacement therapy for lysosomal diseases: lessons from 20 years of experience and remaining challenges. Annu Rev Genomics Hum Genet. 2012; 13:307-335.
Diez-Fernandez C, Rüfenacht V, Gemperle C, Fingerhut R, Häberle J. Mutations and common variants in the human arginase 1 (ARG1) gene: Impact on patients, diagnostics, and protein structure considerations. Hum Mutat. 2018; 39 (8): 1029-1050.
Franzoi A E A, Patti M M, Dal Magro D D, et al. The main neurological dysfunctions in hyperargininemia-literature review. Int J Neurol Neurother. 2018; 5 (1).
Geiger R, Strasak A, Treml B, et al. Six-minute walk test in children and adolescents. J Pediatr. 2007; 150 (4): 395-399.
Häberle J, Boddaert N, Burlina A, et al. Suggested guidelines for the diagnosis and management of urea cycle disorders. Orphanet J Rare Dis. 2012; 7:32.
Häberle J, Burlina A, Chakrapani A, et al. Suggested guidelines for the diagnosis and management of urea cycle disorders: First revision. J Inherit Metab Dis. 2019; 42 (6): 1192-1230.
Huemer M, Carvalho D R, Brum J M, et al. Clinical phenotype, biochemical profile, and treatment in 19 patients with arginase 1 deficiency. J Inherit Metab Dis. 2016; 39 (3): 331-340.
Jain-Ghai S, Nagamani S C S, Blaser S, Siriwardena K, Feigenbaum A. Arginase I deficiency: severe infantile presentation with hyperammonemia: more common than reported? Mol Genet Metab 2011; 104 (1-2): 107-111.
Lambert M A, Marescau B, Desjardins M, et al. Hyperargininemia: intellectual and motor improvement related to changes in biochemical data. J Pediatr. 1991; 118 (3): 420-424.
Maher C A, Williams M T, Olds T S. The six-minute walk test for children with cerebral palsy. Int J Rehabil Res. 2008; 31 (2): 185-8. doi: 10.1097/MRR.0b013e32830150f9.
Marescau B, De Deyn P P, Lowenthal A, et al. Guanidino compound analysis as a complementary diagnostic parameter for hyperargininemia: follow-up of guanidino compound levels during therapy. Pediatr Res. 1990; 27 (3): 297-303.
Oeffinger, D, Bagley A, Rogers S, et al. Outcome tools used for ambulatory children with cerebral palsy: responsiveness and minimum clinically important differences. Dev Med Child Neurol. 2008; 50 (12); 918-925.
Prasad A N, Breen J C, Ampola M G, Rosman N P. Argininemia: a treatable genetic cause of progressive spastic diplegia simulating cerebral palsy: case reports and literature review. J Child Neurol. 1997; 12 (5): 301-309.
Schlune A, vom Dahl S, Häussinger D, Ensenauer R, Mayatepek E. Hyperargininemia due to arginase I deficiency: the original patients and their natural history, and a review of the literature. Amino Acids. 2015; 47:1751-1762.
Sin Y Y, Baron G, Schulze A, Funk C D. Arginase-1 deficiency. J Mol Med (Berl). 2015; 93 (12): 1287-1296.
Sparrow S S, Cicchetti V D, Balla A D. Vineland adaptive behavior scales. 2nd edition. American Guidance Service; Circle Pines, MN; 2005.
Summar M L, Koelker S, Freedenberg D, Le Mons C, Häberle J, Lee H S. The incidence of urea cycle disorders. Mol Genet Metab. 2013; 110 (1-2): 179-180.
Teplan M. Fundamentals of EEG Measurement. Measurement Science Review, Volume 2, Section 2, 2002.
Waisbren S E, Cuthbertson D, Burgard P, et al. Biochemical markers and neuropsychological functioning in distal urea cycle disorders. J Inherit Metab Dis. 2018; 41 (4): 657-667.
Wang Y C, Magasi S R, Bohannon R W, et al. Assessing dexterity function: a comparison of two alternatives for the NIH Toolbox. J Hand Ther. 2011 Oct-Dec; 24 (4): 313-320.
Wong D, Cederbaum S, Crombez E A. In: Adam M P, Ardinger H H, Pagon R A, Wallace S E, Bean L J H, Stephens K, Amemiya A, editors. GeneReviews® [Internet]. Seattle (WA): University of Washington, Seattle; 1993-2019. 2004 Oct. 21 [updated 2014 Aug. 28].
Wu G, Morris S M. Arginine metabolism: nitric oxide and beyond. Biochem J. 1998; 336 (Pt 1): 1-17. Retrieved from https://www.ncbi.nlm.nih.gov/pubmed/9806879.
Yu J Y, Pearl P L. Metabolic causes of epileptic encephalopathy. Epilepsy Res Treat. 2013. doi: 10.1155/2013/124934.
ATS Statement Guidelines for the Six-Minute Walk, Am. J Respir. Crit. Care Med., 166:111-1 17, 2002
Cheng et al., Cancer Res. 67:309-17, 2007
Dillon et al, “Biochemical characterization of the arginine degrading enzymes arginase and arginine deiminase and their effect on nitric oxide production”, Med. Sci. Monit., 8 (7): BR248-253 (2002)
Harris et al, Clin. Pharmacokinet. 40 (7): 539-51, 2001
Lopez et al, FEBSJ. 272:4540-48, 2005
Luneburg, N. et al., “Reference intervals for plasma L-arginine and the L-arginine: asymmetric dimethylarginine ratio in the Framingham Offspring Cohort”, J. Nutr. 141 (12): 2186-2190 (2011).
Marescau et al. “Guanidino compound analysis as a complementary diagnostic parameter for hyperargininemia: Follow-up of guanidino compound levels during therapy”, Pediatric. Res. 27 (3): 297-303 (1990)
Marescau et al., “The pathobiochemistry of uremia and hyperargininemia further demonstrates a metabolic relationship between urea and guanidinosuccinic acid”, 1992 41 (9): 1021-1024 Oeffmger et al., “Outcome tools used for ambulatory children with cerebral palsy: responsiveness and minimum clinically important differences”, Dev. Med. Chile Neurol., 2008; 50 (12): 918-925
Remington, The Science and Practice of Pharmacy, 19th ed., Gennaro, ed., Mack Publishing Co., Easton, PA 1995
Savoca et al, Cancer Biochem. Biophys. 7:261-268, 1984
U.S. Pub. 20170240922; U.S. Pub. 20170283830; U.S. Pub. 20170224843; U.S. Pub. 20170191078; U.S. Pub. 20160095884; U.S. Pat. Nos. 8,398,968; 8,440,184; U.S. Pub. 20160095884; and U.S. Pub. 20140154797.

1. A method of treating Arginase 1 (ARG1) deficiency (ARG1-D) in a subject, the method comprising:
administering human arginase 1 to the subject,
wherein the human arginase 1 comprises a cobalt metal cofactor, and
wherein the human arginase 1 is administered weekly at a dose of from about 0.05 mg/kg to about 0.2 mg/kg of subject weight, and
after one or more weekly doses of the human arginase 1, increasing a daily amount of total protein intake in the subject as compared to when said subject is on an ARG1-D protein restricted diet.

2.-34. (canceled)

35. The method of claim 1, comprising increasing the daily amount of total protein intake with at least 5%, 10%, 15%, 20%, 25% or 45% as compared to when on the protein restricted diet.

36. The method of claim 1, wherein increasing the daily amount of total protein intake comprises increasing the daily amount of natural protein intake such that the natural protein intake is at least 45%, 50%, 55%, 60%, 65% or 70% of the total protein intake.

37. The method of claim 1, further comprising, in addition to increasing the daily amount of total protein intake, reducing a daily intake of essential amino acids in the subject as compared to when on the protein restricted diet.

38. The method of claim 1, wherein the subject is an adult and the amount of total protein intake is increased to more than 40 g/day.

39. The method of claim 1, wherein the human arginase 1 is a pegylated human arginase 1.

40. The method of claim 1, wherein the human arginase 1 is administered subcutaneously, intravenously, intrathecally, intramuscularly, intratumorally, and/or intraperitoneally to the subject.

41. The method of claim 1, wherein the subject is co-administered an ammonia scavenger.

42. The method of claim 41, wherein the ammonia scavenger is a sodium benzoate and/or a glycerol phenylbutyrate.

43. The method of claim 1, wherein after one or more weekly doses of the human arginase 1, the subject's total consumed calories, total consumed protein/day, and total consumed natural protein, is increased by at least 15%.

44. A combination therapy for use in treating a subject with Arginase 1 (ARG1) deficiency (ARG1-D), comprising:
human arginase 1 in a weekly dose of from about 0.05 mg/kg to about 0.2 mg/kg of subject weight, said arginase 1 comprising a cobalt metal cofactor, and
a daily amount of total protein that is higher than a daily amount of total protein for a subject with ARG1-D on a protein restricted diet.

45. The combination therapy of claim 44, wherein the daily amount of total protein comprises an amount of natural protein that is at least 5%, 10%, 15%, 20%, 25% or 45% of the daily amount of total protein.

46. The combination therapy of claim 44, wherein the daily amount of total protein is at least 45%, 50%, 55%, 60%, 65% or 70% of the total protein intake.

47. The combination therapy of claim 44, wherein the subject is an adult and the daily amount of total protein is greater than 40 g/day.

48. The combination therapy of claim 44, wherein the human arginase 1 is a pegylated human arginase 1.

49. The combination therapy of claim 44, further comprising an ammonia scavenger.

50. The combination therapy of claim 49, wherein the ammonia scavenger is a sodium benzoate and/or a glycerol phenylbutyrate.

51. The combination therapy of claim 44, further comprising a salt and/or a glycerol.

52. The combination therapy of claim 51, wherein the salt is NaCl, K₂HPO₄, KH₂PO₄, or any combination thereof.
